(19) United States
(12) Patent Application Publication
(10) Pub. No.: US 2025/0236611 A1
(43) Pub. Date: Jul. 24, 2025

(54) PYRAZOLE COMPOUNDS AND USES THEREOF

(71) Applicant: ALCON INC., Fribourg (CH)

(72) Inventors: David Archer Ellis, Durham, NC (US); Heeren M. Gordhan, Durham, NC (US); Cynthia L. Lichorowic, Durham, NC (US); Jill M. Sturdivant, Chapel Hill, NC (US); Mitchell A. deLong, Chapel Hill, NC (US)

(21) Appl. No.: 18/699,188

(22) PCT Filed: Oct. 6, 2022
(86) PCT No.: PCT/US2022/077707

§ 371 Date: Apr. 5, 2024

Related U.S. Application Data
(60) Provisional application No. 63/253,026, filed on Oct. 6, 2021.

(51) Int. Cl. C07D 403/14; A61K 31/506; A61K 31/5377; A61K 31/675; C07D 403/04; C07D 417/14; C07F 9/6558

(52) U.S. Cl. CPC C07D 403/14; A61K 31/506; A61K 31/5377; A61K 31/675; C07D 403/04; C07D 417/14; C07F 9/65583

(57) ABSTRACT

Provided herein are pyrazole compounds. In particular, provided herein are compounds that affect the function of kinases in a cell and that are useful as therapeutic agents or with therapeutic agents. The compounds provided herein are useful in the treatment of a variety of diseases and conditions including cardiovascular diseases, JAK-associated diseases, such as diseases characterized by abnormal growth, such as cancers, and inflammatory diseases, or eye diseases such as glaucoma. Also provided herein are compositions comprising pyrazole compounds.

### RELATED APPLICATIONS

This application is a national stage entry under 35 U.S.C. § 371 of international patent application no. PCT/US2022/077707, filed Oct. 6, 2022, which claims priority of U.S. provisional patent application No. 63/253,026, filed Oct. 6, 2021, the entire content of each of which is incorporated herein by reference.

### BACKGROUND

The present disclosure relates to pyrazole compounds, which affect the function of phosphotransferases such as protein kinases, and are useful as therapeutic agents or with therapeutic agents.

Protein kinases play roles in biological pathways including adaptive immunity, adhesion and migration, angiogenesis, apoptosis, bone development, bone growth, bone remodeling, cancer, cell cycling, cellular proliferation, differentiation, immunity, metabolism, and transcription. Kinases may be classified by their target into Serine/Threonine kinases and Tyrosine kinases. Tyrosine kinases include receptor and non-receptor tyrosine kinases. Janus kinases (JAKs) are non-receptor tyrosine kinases, which include the four members JAK1, JAK2, JAK3, and TYK2.

The Janus kinase (JAK), signal transducer of activation (STAT) pathway is a signaling pathway used by cytokines, interferons, growth factors, and related molecules. The JAK/STAT pathway provides mechanism for extracellular factors to control gene expression, and thereby regulate cell growth and differentiation. Mutations and polymorphisms of components of the JAK/STAT pathway, as well as elevated or decreased levels of JAK/STAT-utilizing cytokines, are relevant to a variety of human diseases, such as cancers and immune-related conditions. For example, mutations or polymorphisms in Type 1 and II cytokine receptors, JAK kinases, STAT proteins, and JAK/STAT regulatory proteins such as phosphotyrosine phosphatases, SOCS proteins, PIAS proteins have been reported in a variety of diseases. When dysregulated, JAK-mediated responses can positively or negatively affect cells leading to over-activation and malignancy or immune and hematopoietic deficiencies, respectively, and suggests the utility for use of inhibitors of JAK kinases. The JAK/STAT signaling pathway is involved in a variety of hyperproliferative and cancer-related processes including cell-cycle progression, apoptosis, angiogenesis, invasion, metastasis, and evasion of the immune system. In addition, the JAK/STAT signaling pathway is important in the genesis and differentiation of hematopoietic cells and regulating both pro-inflammatory and anti-inflammatory, and immune responses.

In view of the role the JAK/STAT pathway plays in disease states, there remains a need for ligands that inhibit or modulate the activity of JAKs.

### SUMMARY

Provided herein are compounds of Formula 1:

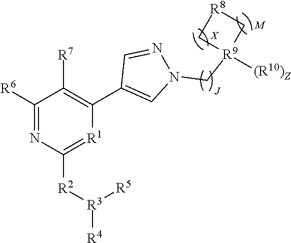

or a pharmaceutically acceptable salt thereof.

Also provided herein are compounds of Formula 2:

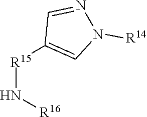

or a pharmaceutically acceptable salt thereof.

Also provided herein are compounds of Formula 3:

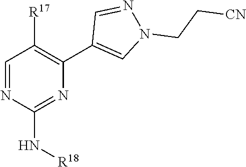

or a pharmaceutically acceptable salt thereof.

Also provided herein are the following compounds:

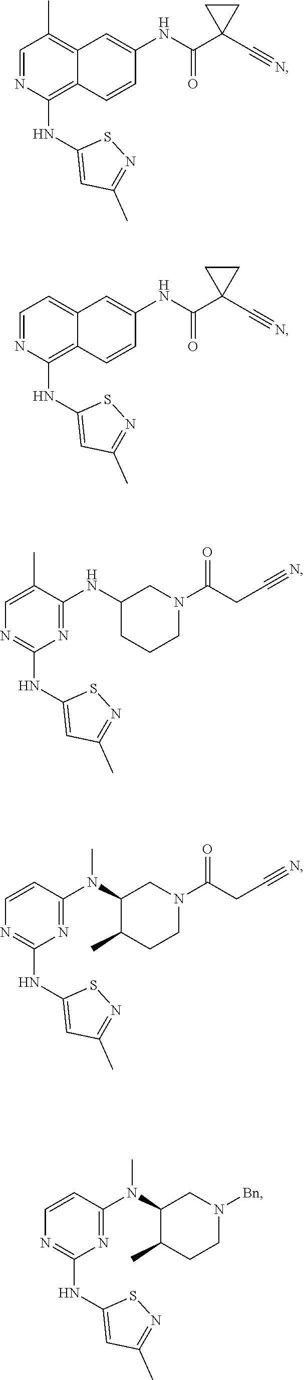 [SMILES: Cc1cc(Nc2ncc(C)c3cc(NC(=O)C4(C#N)CC4)ccc23)sn1 ; Cc1cc(Nc2nccc3cc(NC(=O)C4(C#N)CC4)ccc23)sn1 ; Cc1cc(Nc2ncc(C)c(NC3CCCN(C(=O)CC#N)C3)n2)sn1 ; Cc1cc(Nc2nccc(N(C)[C@H]3CN(C(=O)CC#N)CC[C@H]3C)n2)sn1 ; Cc1cc(Nc2nccc(N(C)[C@H]3CN(Cc4ccccc4)CC[C@H]3C)n2)sn1]

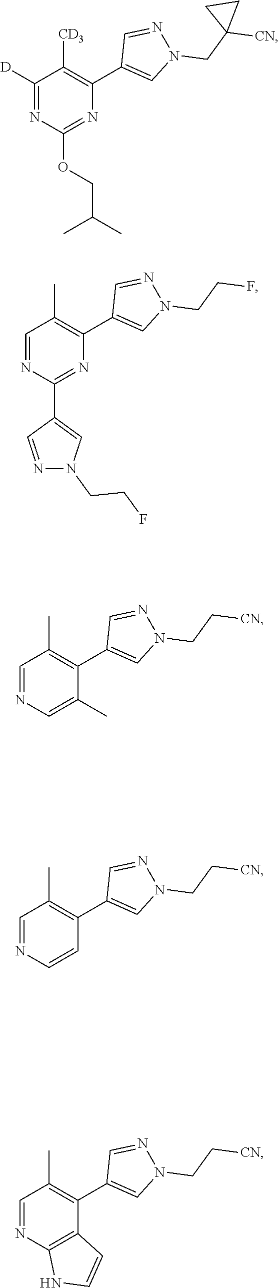

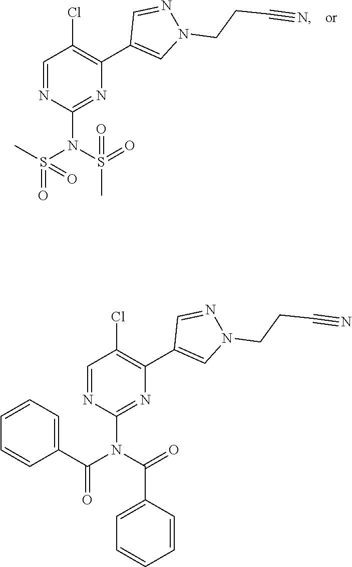

or pharmaceutically acceptable salts thereof.

Also provided herein are uses of the compounds and compositions provided herein, such as for the treatment of diseases.

### BRIEF DESCRIPTION OF THE FIGURES

FIG. 1 shows a first general scheme for the preparation of compounds provided herein.

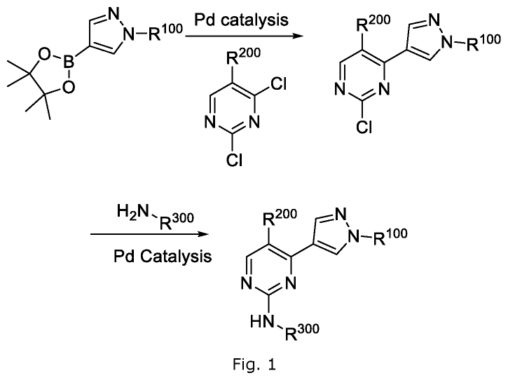

FIG. 2 shows a second general scheme for the preparation of compounds provided herein.

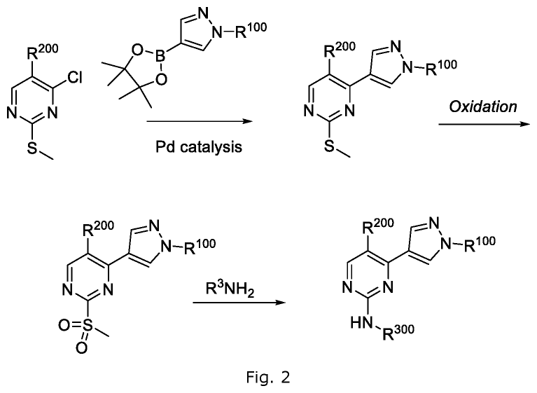

FIG. 3 shows a second general scheme for the preparation of compounds provided herein.

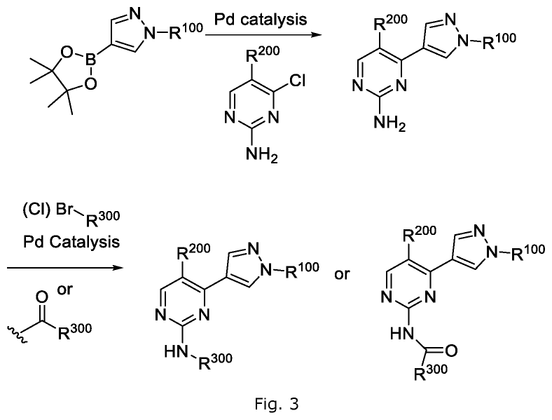

### DETAILED DESCRIPTION

### Definitions

The terms “a” and “an” and “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the disclosure and does not pose a limitation on the scope of the disclosure otherwise claimed. No language in the specification should be construed as indicating any non-claimed element essential to the practice of the disclosure.

The term “about,” when referring to a measurable value such as an amount, a temporal duration, and the like, refers to variations of ±20%, as such variations are appropriate to perform the disclosed methods. In some embodiments, about refers to variations of ±10%, ±5%, ±1%, or ±0.1% from the specified value.

The term “alkyl” refers to a saturated aliphatic hydrocarbon radical including straight chain or branched chain groups. “Alkyl” may be exemplified by groups such as methyl, ethyl, n-propyl, isopropyl, n-butyl and the like.

The term “aryl” refers to an aromatic carbocyclic radical that may be monocyclic, or polycyclic having fused or non-fused rings. In some embodiments, aryl is phenyl, biphenyl, naphthyl, tetrahydronaphthyl, indanyl, or indenyl.

The term “Cn-m” refers to groups containing the number of carbon atoms in the range indicated by the integers “n” and “m.”

The term “cycloalkyl” refers to a radical of a saturated carbocyclic ring compound being monocyclic, or polycyclic having fused or nonfused rings. Examples of C₃₋₈-cycloalkyl include, but are not limited to, cyclopropyl, cyclobutyl, cyclopentyl, cyclohexyl, cyclopentyl and cyclooctyl.

The term “haloalkyl” refers to a saturated aliphatic hydrocarbon radical including straight chain or branched chain groups having one or more halogens (e.g., 1, 2, 3, 4, 5, 6, 7, 8, 9, 10 or more) in the hydrocarbon.

The terms “halogen” or “halo” refer to fluoro, chloro, bromo, or iodo moieties. In some embodiments, the halogen is fluoro, chloro, or bromo. In some embodiments, the halogen is fluoro or chloro.

The term “heteroalkyl” refers to a saturated aliphatic hydrocarbon radical including straight chain or branched chain groups having one or more heteroatoms (e.g., 1, 2, 3, 4, 5, 6, 7, 8, 9, 10 or more) in the hydrocarbon.

The term “heteroaryl” refers to an aromatic carbocyclic radical having one or more heteroatoms (e.g., 1, 2, 3, 4, 5, 6, 7, 8, 9, 10 or more) in the carbocyclic ring.

The terms “heteroatom” or “hetero” refer, independently for each occurrence, to N, O, S, P, or Si. In some embodiments, each heteroatom is, independently, selected from N, O, or S.

The term “heterocycloalkyl” refers to a radical of a monocyclic or polycyclic saturated carbocyclic ring compound having one or more heteroatoms (e.g., 1, 2, 3, 4, 5, 6, 7, 8, 9, 10 or more) in the carbocyclic ring.

The phrase “JAK-associated disease” refers to any disease, disorder, or condition that is directly or indirectly linked to expression or activity of a JAK, including overexpression, or abnormal activity levels, or a disease, disorder, or condition that can be prevented, ameliorated, rendered undetectable by conventional means, neutralized, or obviated by modulating JAK activity.

The use of the term “or” in the claims is used to mean “and/or” unless explicitly indicated to refer to alternatives only or the alternatives are mutually exclusive, although the disclosure supports a definition that refers to only alternatives and “and/or.”

The phrase “pharmaceutically acceptable carrier” refers to a carrier that is useful for the preparation of a pharmaceutical composition that is: generally compatible with the other ingredients of the composition; not deleterious to the recipient; and neither biologically nor otherwise undesirable. “A pharmaceutically acceptable carrier” includes both one and more than one carrier. Some embodiments include carriers for topical, ocular, parenteral, intravenous, intraperitoneal intramuscular, sublingual, nasal, or oral administration. “Pharmaceutically acceptable carrier” also includes agents for preparation of aqueous dispersions and sterile powders for injection or dispersions.

The phrase “pharmaceutically acceptable salts” refers to an ionizable therapeutic agent that has been combined with a counter-ion to form a neutral complex. Included are salts of the compounds prepared with relatively nontoxic acids or bases, depending on the particular substituents found on the compounds provided herein. Neutral forms of the compounds may be regenerated by contacting the salt with a base or acid and isolating the parent compound in a conventional manner. Lists of suitable salts are found in Remington's Pharmaceutical Sciences, 17th ed., Mack Publishing Company, Easton, Pa., 1985, p. 1418, and Berge et al., 1977, “Pharmaceutically Acceptable Salts.” J. Pharm. Sci., 66, pp. 1-19, each of which is incorporated herein by reference in its entirety.

Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “therapeutically effective amount” refers to a dosage of the compounds or compositions effective for influencing, reducing, or inhibiting the activity of or preventing activation of a kinase. This term may also refer to an amount effective at bringing about a desired in vivo effect in a subject, preferably, a human.

“Treatment” refers to any type of intervention in a subject or a cell provided as a means to alter the natural course of a disease or pathology in the individual or cell. Treatment includes, but is not limited to, administration of one or more compounds provided herein or a pharmaceutical composition thereof, and may be performed either prophylactically, or subsequent to the initiation of a pathologic event or contact with an etiologic agent. Treatment includes any desirable effect on the symptoms or pathology of a disease or condition associated with inflammation, among others provided herein.

### Compounds

Provided herein are pyrazole compounds, which are useful as therapeutic agents or with therapeutic agents. In some embodiments, provided herein are compounds of the formula:

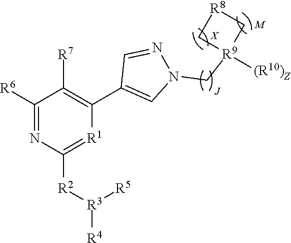

or a pharmaceutically acceptable salt thereof,
wherein
R¹ is N, CH, CF, CCl, or CBr;
R² is NH,

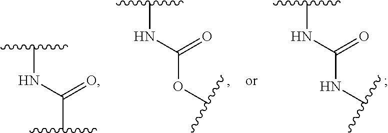 [SMILES: *NC(*)=O ; *NC(=O)O* ; *NC(=O)N*]

R³ is C₆₋₁₀ aryl or C₂₋₁₀ heteroaryl;
R⁴ is H, C₁₋₄ alkyl, C₁₋₄ haloalkyl, F, Cl, Br, I, O(C₁₋₄ alkyl), C₁₋₄ alkyl-OH, CH₂NH₂, CH₂N(C₁₋₄ alkyl)(C₁₋₄ alkyl), Boc, C(O)N(H)(C₁₋₄ alkyl), CH₂N(H)Boc, C(O)O(C₁₋₄ alkyl), C₂₋₈ heterocycloalkyl, CH₂—(C₂₋₈ heterocycloalkyl), (C₂₋₈ heterocycloalkyl)-CH₃, CH₂—(C₂₋₈ heterocycloalkyl)-CH₃, C(O)OH, S(O₂)(C₁₋₄ alkyl), C(O)NH₂, C₁₋₆ heteroalkyl, CH₂OC(O)—(C₆₋₁₀ aryl), (C₂₋₈ heterocycloalkyl)-Boc, (C₁₋₆ heteroalkyl)-(C₂₋₈ heterocycloalkyl)-(C₁₋₄ alkyl), CH₂OC(O)(C₁₋₆ heteroalkyl), CH₂OC(O)CH₂CH₂—(C₂₋₈ heterocycloalkyl), C(O)N(C₁₋₄ alkyl)(C₁₋₄ alkyl), CH₂N(H)C(O)C(CH₃)N(H)Boc, CH₂N(H)C(O)-pyrrolidinyl-Boc, CH₂N(H)C(O)C(CH₃)NH₂, CH₂N(H)C(O)-pyrrolidinyl, CH₂N(H)Boc, CH₂N(H)C(O)CH₂N(CH₃)₂, CH₂N(H)C(O)C(N(H)Boc)CH₂CH₂CH₂-guanidinyl(Boc)₂, CH₂N(H)C(O)C(NH₂)CH₂CH₂CH₂-guanidinyl, (C₂₋₈ heterocycloalkyl)-N(C₁₋₄ alkyl)(C₁₋₄ alkyl), or a C₅₋₁₀ spiroheterocycloalkyl;
R⁵ is H, F, Cl, Br, I, C₁₋₄ alkyl, C₁₋₄ haloalkyl, C₁₋₄ O-alkyll, C₁₋₄ alkyl-OH, ═O, or C(O)O(C₁₋₄ alkyl);
or R⁴ and R⁵ together form —O—N═C(H)—, —C(H₂)—N(H)—C(O)—, —C(O)—N(CH₃)—C(O)—, —C(H₂)—N(CH₃)—C(H₂)—, or ═C(H)—C(H)═N—;
R⁶ is H and R⁷ is H, F, Cl, Br, I, C₁₋₄ alkyl, C₁₋₄ haloalkyl, C₁₋₄ O-alkyll, or C₃₋₆ cycloalkyl;
or R⁶ and R⁷ together form a C₂₋₅ alkylene or a C₂₋₅ heteroalkylene;
R⁸ is a bond, NH, O, CH₂, N(Boc), N(CH₂F), N(CHF₂), N(CF₃), N(CH₂CH₂F), N(CH₂CHF₂), N(CH₂CF₃), N(CH₂CN), or N(CH₂CH₂CN);
R⁹ is N and Z is 0;
or R⁹ is C and Z is 1;
R¹⁰ is H, CH₂F, CHF₂, CF₃, CH₂Cl, CHCl₂, CCl₃, CH₂Br, CH₂I, F, Cl, Br, I, C₁₋₄ O-alkyll, C₁₋₃ alkylene-OH, CN, CH₂CN, CH₂CH₂CN, C(O)OH, OH, NH₂, N(H)CH₃, N(CH₃)₂, CH₂NH₂, CH₂N(H)CH₃, CH₂N(CH₃)₂, or N(Boc)CH₃;
J is 0, 1, or 2;
X is 0, 1, or 2;
M is 1 or 2; and
each hydrogen, independently, may be replaced with deuterium.

In some embodiments, 1, 2, 3, 4, 5, 6, 7, 8, 9, 10, 11, or 12 hydrogens, independently, are replaced with deuterium.

In some embodiments, J is 1 or 2. In some embodiments: J is 1 or 2; R⁹ is C; and Z is 1.

In some embodiments, X is 0. In some embodiments, X is 1 or 2. In some embodiments: X is 1; M is 1; and R⁸ is NH, O, CH₂, N(Boc), N(CH₂F), N(CHF₂), N(CF₃), N(CH₂CH₂F), N(CH₂CHF₂), N(CH₂CF₃), N(CH₂CN), or N(CH₂CH₂CN). In some embodiments: X is 1; M is 1; R⁸ is a bond; R⁹ is C; and Z is 1.

In some embodiments, R¹ is N, CH, or CF. In some embodiments, R¹ is N. In some embodiments, R¹ is CH or CF.

In some embodiments, R⁶ is H and R⁷ is H, F, Cl, CH₃, CH₂CH₃, OCH₃, or cyclopropyl. In some embodiments, R⁶ is H and R⁷ is H, F, Cl, or CH₃.

In some embodiments

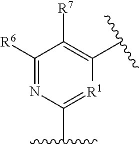

is R¹² as defined below.

In some embodiments

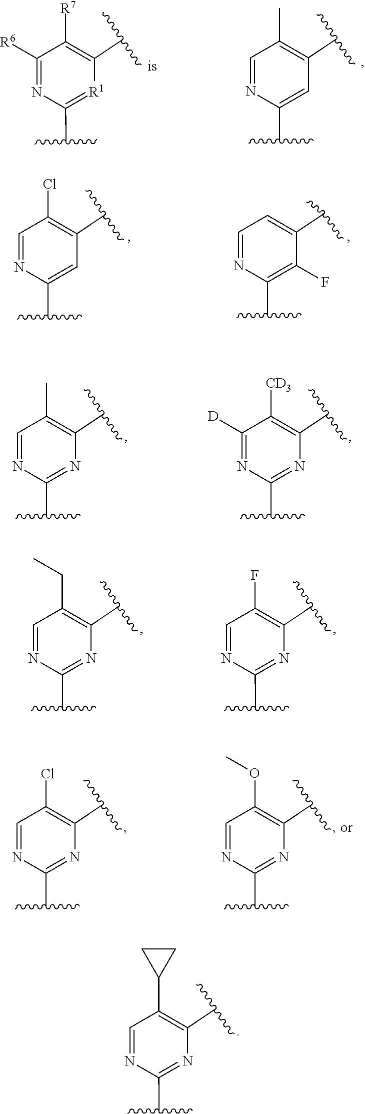

In some embodiments, R⁸ is a bond, NH, O, CH₂, N(CH₂F), or N(CH₂CN). In some embodiments, R⁸ is a bond. In some embodiments, R⁸ is NH, O, N(CH₂F), or N(CH₂CN).

In some embodiments, R⁹ is C and Z is 1. In some embodiments, R⁹ is N and Z is 0.

In some embodiments, R¹⁰ is H, CH₂F, CHF₂, F, Cl, CH₂OH, CN, CH₂CN, C(O)OH, N(H)CH₃, or CH₂N(CH₃)₂.

In some embodiments

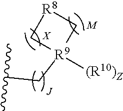

is R¹¹ as defined below.

In some embodiments

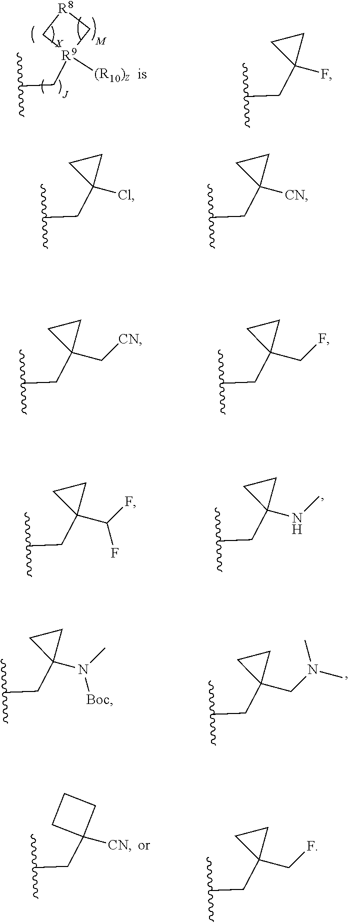

In some embodiments

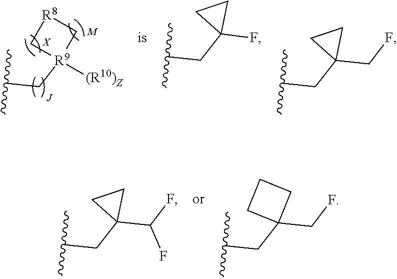

In some embodiments, R⁴ is H, CH₃, CH₂NH₂, CH₂N(CH₃)₂, Boc, C(O)N(H)CH₃, CH₂N(H)Boc, piperazinyl, C(O)OCH₃, piperazinyl-CH₃, C(O)OH, S(O₂)CH₃, C(O)NH₂, morpholinyl, CH₂OH, Cl, OCH₃, CH₂-morpholinyl, CH₂OCH₃, CH₂-piperazinyl-CH₃, CH₂CH₂N(CH₃)₂, CH₂OC(O)-phenyl, CH₂N(H)CH₃, piperazinyl-Boc, CH₂OCH₂CH₂OCH₃, CH₂OC(O)CH₂CH₂-piperazynyl-CH₃, CH₂OC(O)CH₂CH₂N(CH₃)₂, CH₂OC(O)CH₂CH₂-morpholinyl, C(O)N(CH₃)₂, CH₂N(H)C(O)C(CH₃)N(H)Boc, CH₂N(H)C(O)-pyrrolidinyl-Boc, CH₂N(H)C(O)C(CH₃)NH₂, CH₂N(H)C(O)-pyrrolidinyl, CH₂N(H)Boc, CH₂N(H)C(O)CH₂N(CH₃)₂, CH₂N(H)C(O)C(N(H)Boc)CH₂CH₂CH₂-guanidinyl(Boc)₂, CH₂N(H)C(O)C(NH₂)CH₂CH₂CH₂-guanidinyl, CF₃, OCH₂CH₂N(CH₃)₂, OCH₂CH₂OCH₃, azetidinyl-N(CH₃)₂, or 2-oxa-6-azaspiro[3.3]heptanyl.

In some embodiments, R⁴ is H, CH₃, CH₂NH₂, CH₂N(CH₃)₂, C(O)N(H)CH₃, piperazinyl, C(O)OCH₃, piperazinyl-CH₃, C(O)OH, S(O₂)CH₃, C(O)NH₂, morpholinyl, CH₂OH, Cl, OCH₃, CH₂-morpholinyl, CH₂OCH₃, CH₂-piperazinyl-CH₃, CH₂CH₂N(CH₃)₂, CH₂OC(O)-phenyl, CH₂N(H)CH₃, CH₂OCH₂CH₂OCH₃, CH₂OC(O)CH₂CH₂-piperazynyl-CH₃, CH₂OC(O)CH₂CH₂N(CH₃)₂, CH₂OC(O)CH₂CH₂-morpholinyl, C(O)N(CH₃)₂, CH₂N(H)C(O)C(CH₃)NH₂, CH₂N(H)C(O)-pyrrolidinyl, CH₂N(H)Boc, CH₂N(H)C(O)CH₂N(CH₃)₂, CH₂N(H)C(O)C(NH₂)CH₂CH₂CH₂-guanidinyl, CF₃, OCH₂CH₂N(CH₃)₂, OCH₂CH₂OCH₃, azetidinyl-N(CH₃)₂, or 2-oxa-6-azaspiro[3.3]heptanyl.

In some embodiments, R⁴ is CH₃, CH₂NH₂, CH₂N(CH₃)₂, C(O)N(H)CH₃, piperazinyl, C(O)OCH₃, piperazinyl-CH₃, C(O)OH, S(O₂)CH₃, C(O)NH₂, morpholinyl, CH₂OH, Cl, OCH₃, CH₂-morpholinyl, CH₂OCH₃, CH₂-piperazinyl-CH₃, CH₂CH₂N(CH₃)₂, CH₂OC(O)-phenyl, CH₂N(H)CH₃, CH₂OCH₂CH₂OCH₃, CH₂OC(O)CH₂CH₂-piperazynyl-CH₃, CH₂OC(O)CH₂CH₂N(CH₃)₂, CH₂OC(O)CH₂CH₂-morpholinyl, C(O)N(CH₃)₂, CH₂N(H)C(O)C(CH₃)NH₂, CH₂N(H)C(O)-pyrrolidinyl, CH₂N(H)Boc, CH₂N(H)C(O)CH₂N(CH₃)₂, CH₂N(H)C(O)C(NH₂)CH₂CH₂CH₂-guanidinyl, CF₃, OCH₂CH₂N(CH₃)₂, OCH₂CH₂OCH₃, azetidinyl-N(CH₃)₂, or 2-oxa-6-azaspiro[3.3]heptanyl.

In some embodiments: R⁸ is a bond, NH, O, CH₂, or N(CH₂F); X is 1; and M is 1.

In some embodiments, R³ is phenyl, isothiazolyl, pyridinyl, pyrazolyl, pyrimidinyl, pyrazolopyrimidinyl, triazolyl, or isoxazolyl. In some embodiments, R³ is phenyl or isothiazolyl.

In some embodiments, R⁵ is H, F, CH₃, ═O, C(O)OCH₃, or CH₂OH. In some embodiments, R⁵ is H, F, or CH₃. In some embodiments, R⁵ is ═O, C(O)OCH₃, or CH₂OH. In some embodiments, R⁵ is H.

In some embodiments

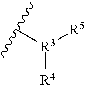

is R¹³ as defined below.

In some embodiments

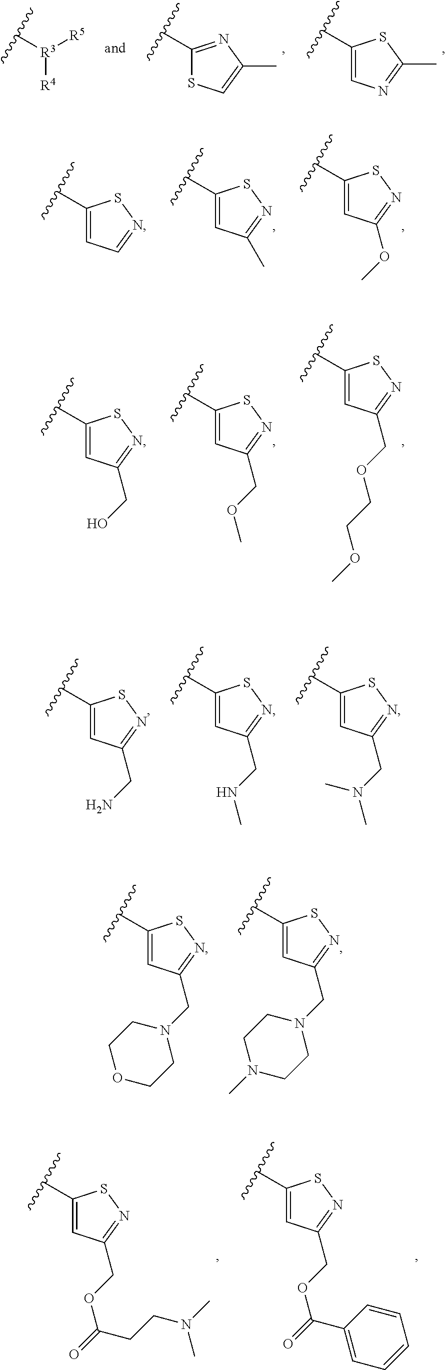

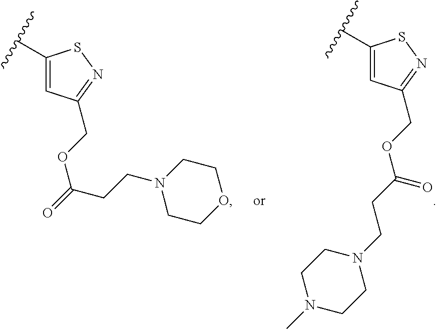 [SMILES: *c1cc(COC(=O)CCN2CCOCC2)ns1 ; *c1cc(COC(=O)CCN2CCN(C)CC2)ns1]

In some embodiments, the compound provided herein is of the formula:

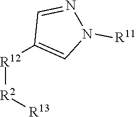

or a pharmaceutically acceptable salt thereof,
wherein
R² is NH,

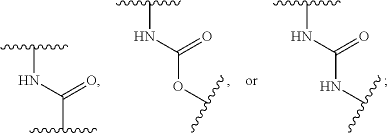 [SMILES: *NC(*)=O ; *NC(=O)O* ; *NC(=O)N*]

R¹¹ is

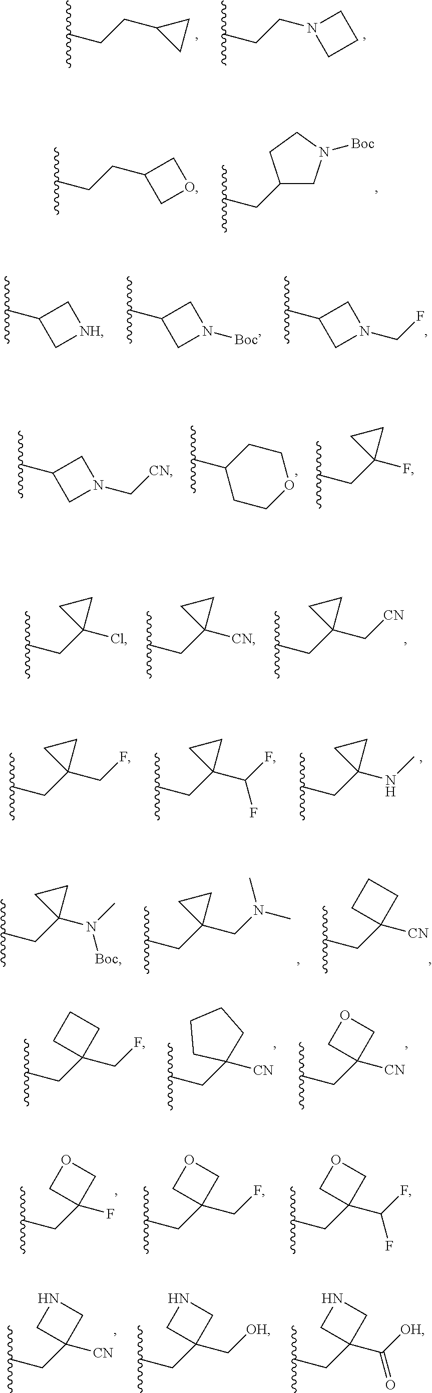

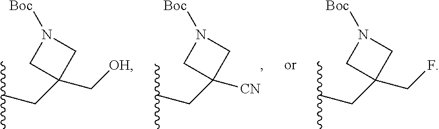 [SMILES: *CC1(CO)CN(C(=O)OC(C)(C)C)C1 ; *CC1(C#N)CN(C(=O)OC(C)(C)C)C1 ; *CC1(CF)CN(C(=O)OC(C)(C)C)C1]

R¹² is

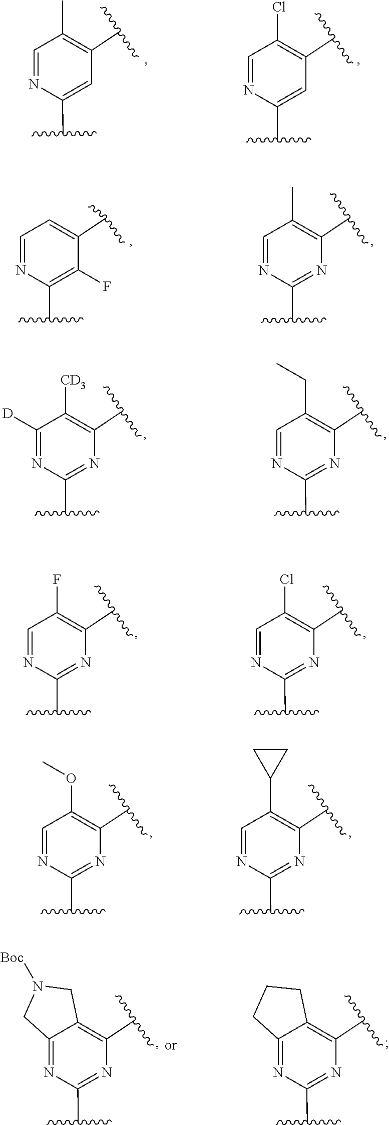

and
R¹³ is

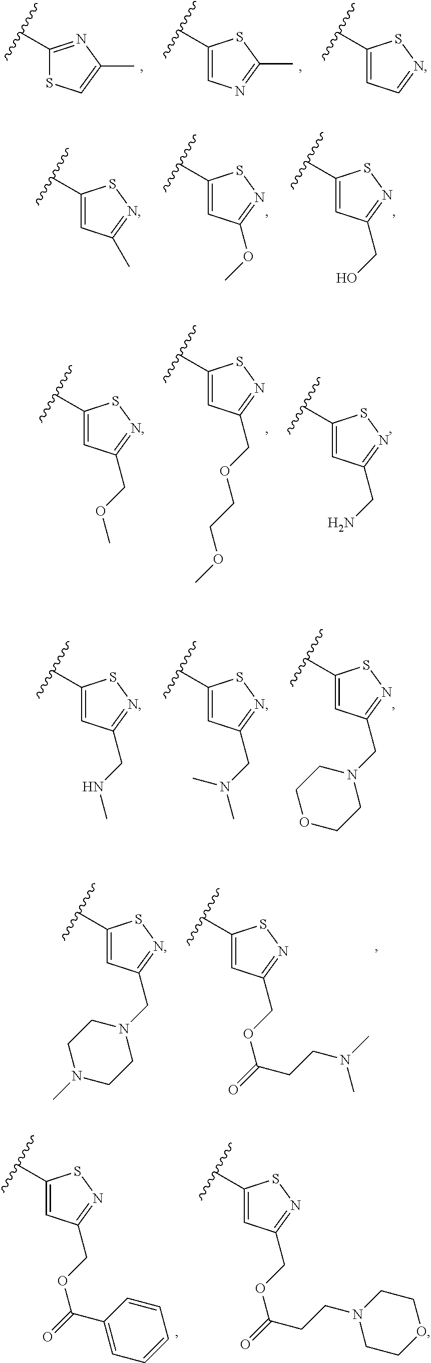

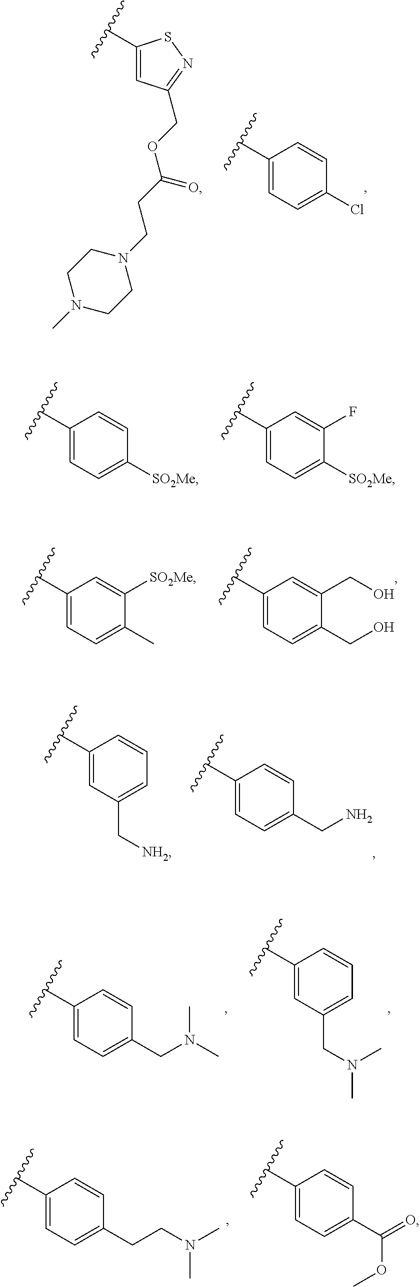

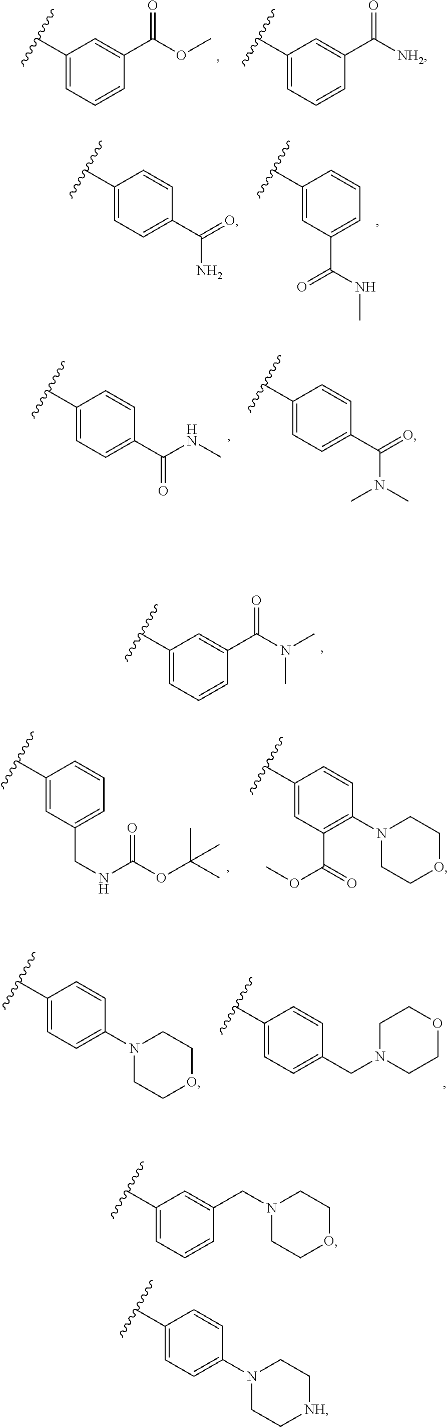

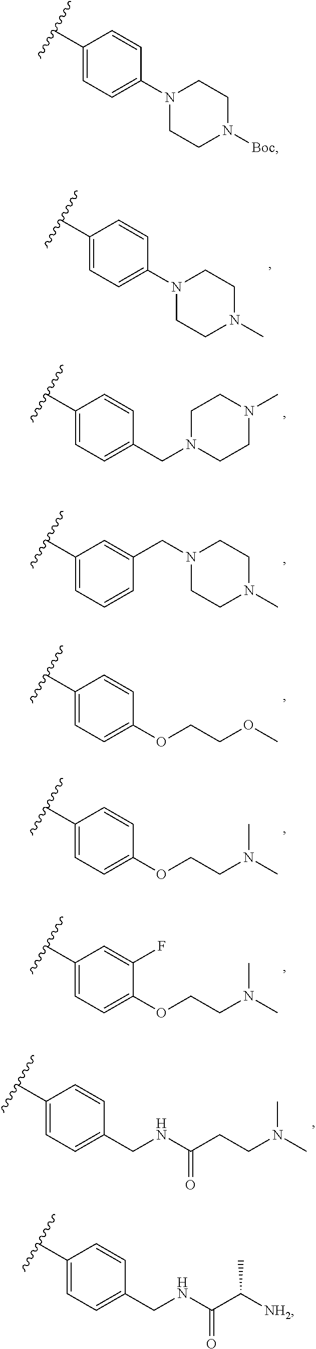

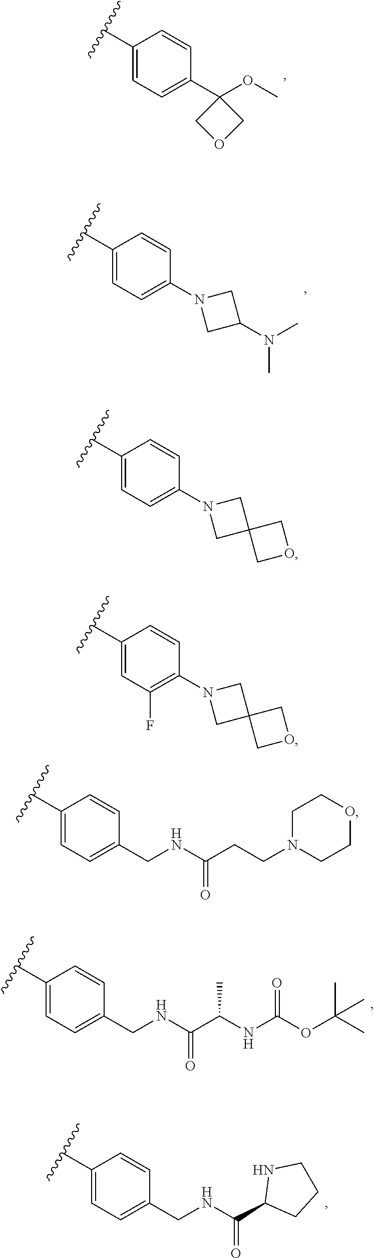

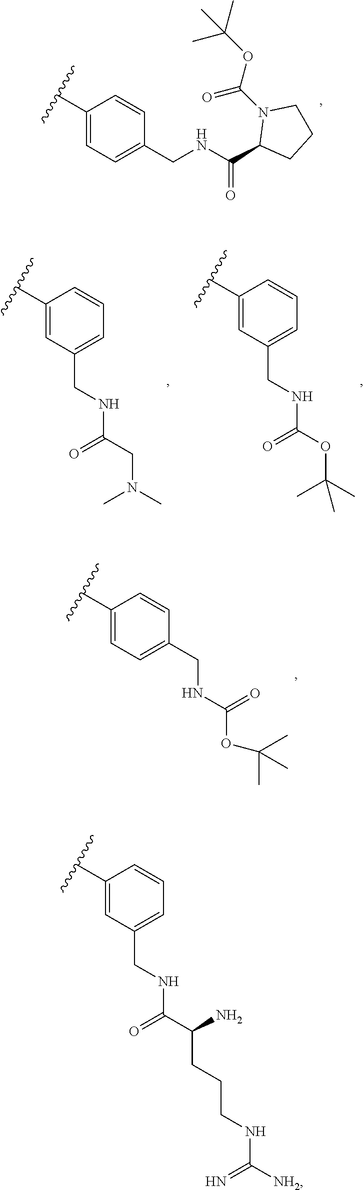 [SMILES: *c1ccc(CNC(=O)[C@@H]2CCCN2C(=O)OC(C)(C)C)cc1 ; *c1cccc(CNC(=O)CN(C)C)c1 ; *c1cccc(CNC(=O)OC(C)(C)C)c1 ; *c1ccc(CNC(=O)OC(C)(C)C)cc1 ; *c1cccc(CNC(=O)[C@@H](N)CCCNC(=N)N)c1]

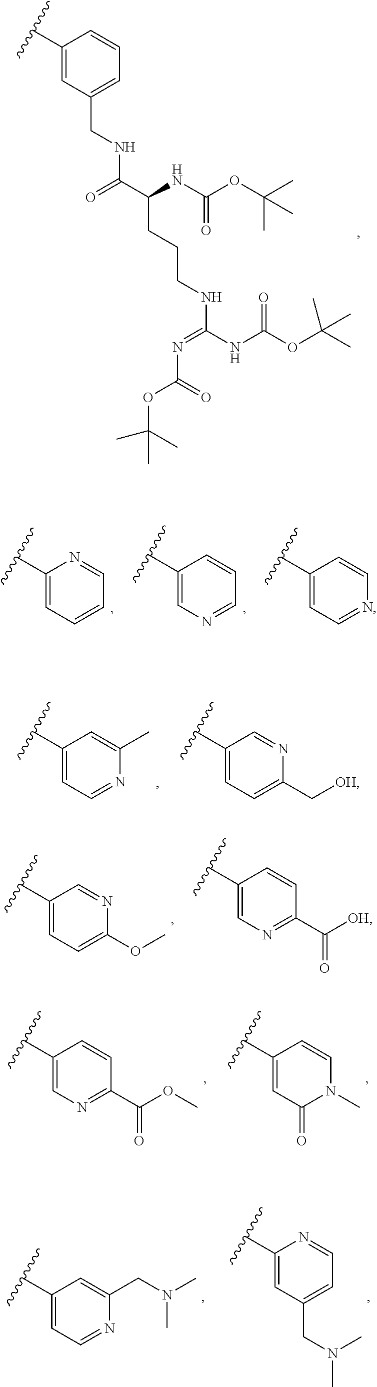

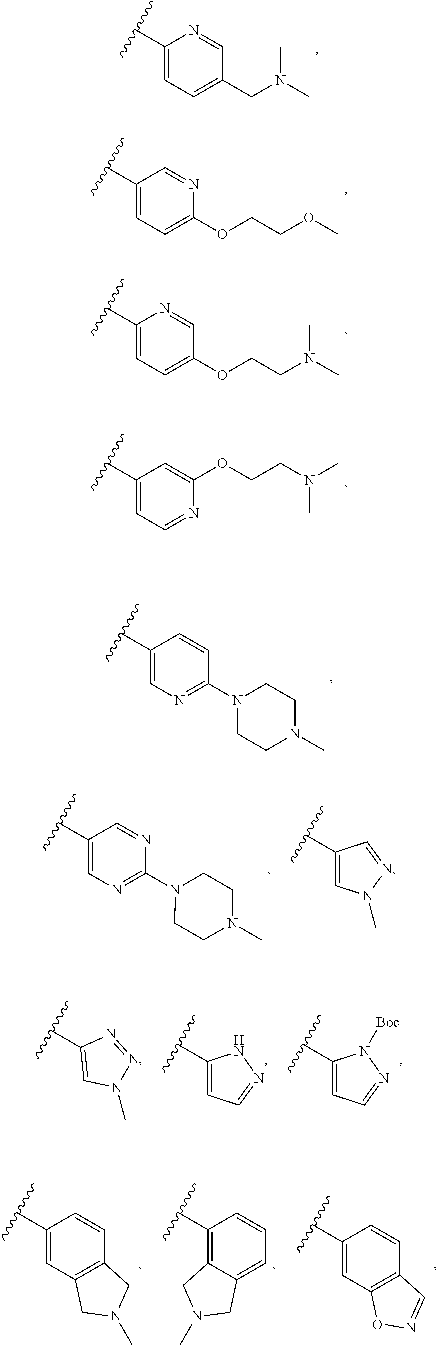

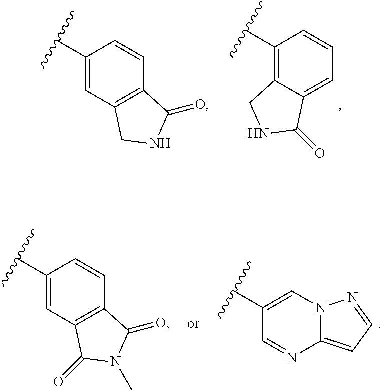 [SMILES: *c1ccc2c(c1)CNC2=O ; *c1cccc2c1CNC2=O ; *c1ccc2c(c1)C(=O)N(C)C2=O ; *c1cnc2ccnn2c1]

In some embodiments, R² is NH,

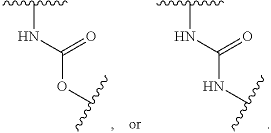 [SMILES: *NC(=O)O* ; *NC(=O)N*]

In some embodiments, R² is NH.

In some embodiments, provided herein are compounds of the formula:

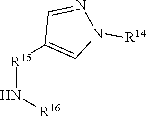

or a pharmaceutically acceptable salt thereof,
wherein
R¹⁴ is

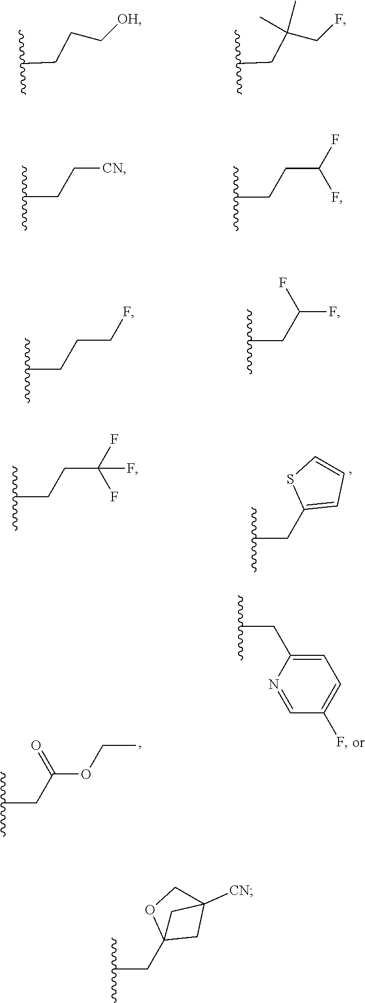

R¹⁵ is

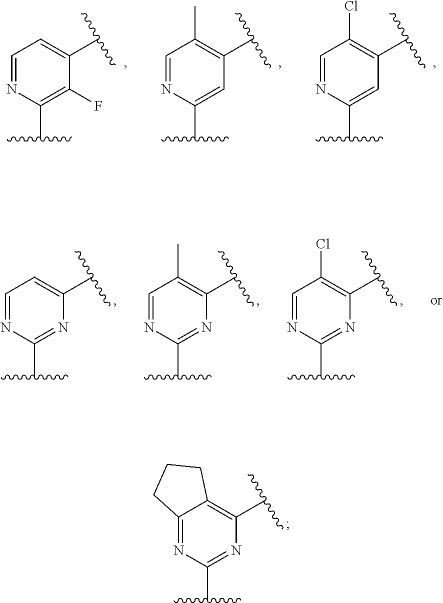

and
R¹⁶ is

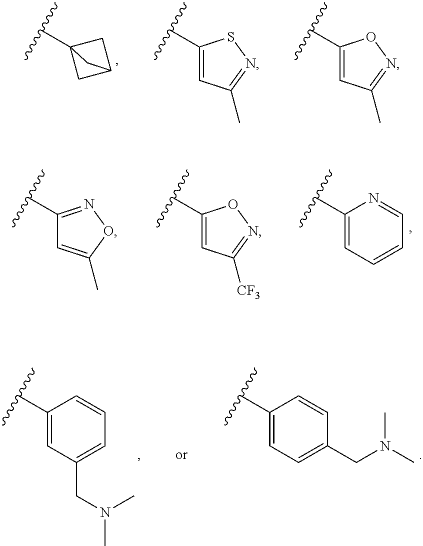

In some embodiments, provided herein are compounds of the formula:

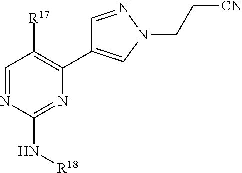

or a pharmaceutically acceptable salt thereof,
wherein
R¹⁷ is H, Cl, or CH₃; and
R¹⁸ is

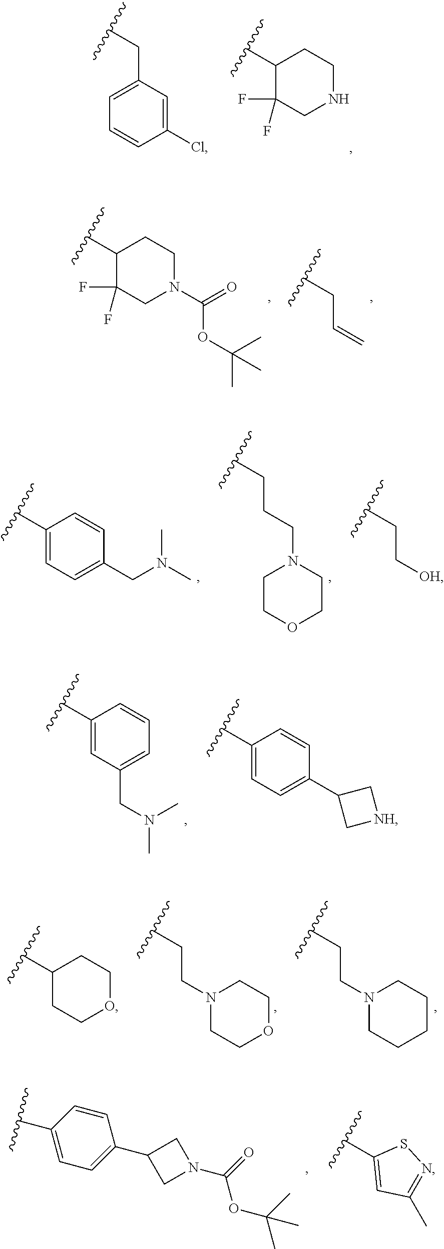

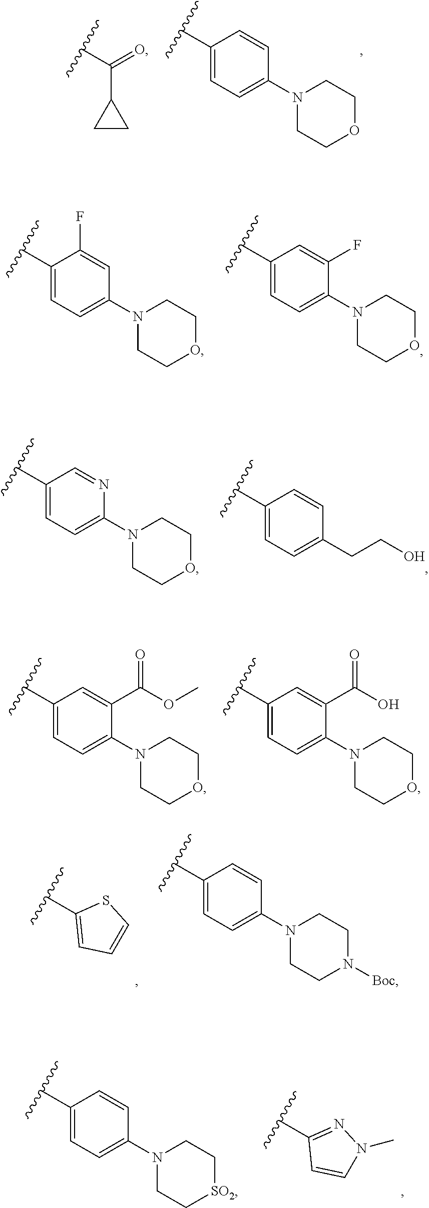

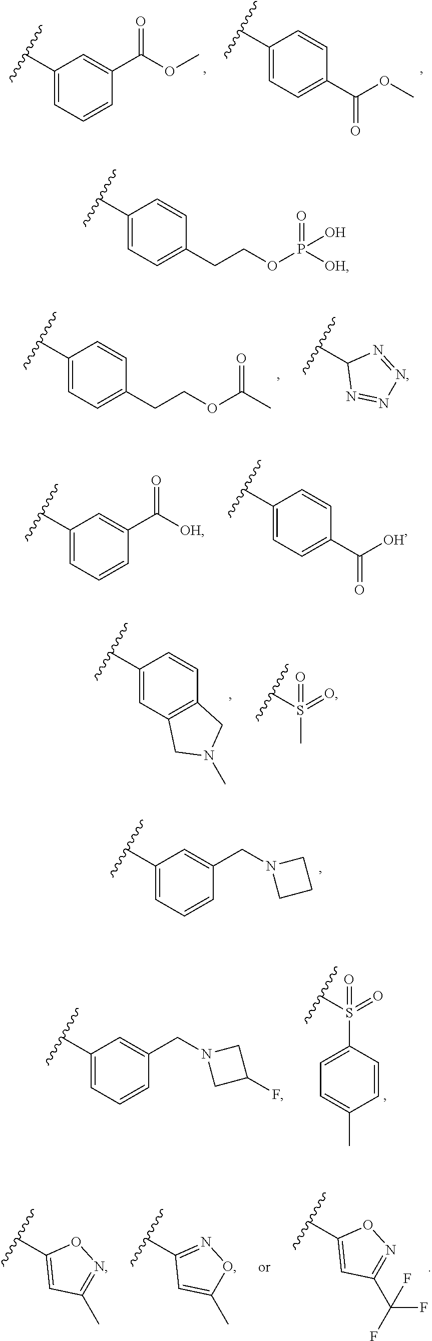

In some embodiments, the compound provided herein is a compound of the formula:

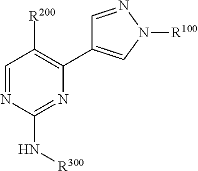

or a pharmaceutically acceptable salt thereof.

In some embodiments, the compound provided herein is a compound of the formula:

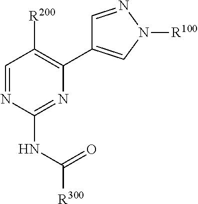

or a pharmaceutically acceptable salt thereof.

In some embodiments, R¹⁰⁰ is a corresponding moiety of a compound or formula provided herein. In some embodiments, R¹⁰⁰ is R¹⁴,

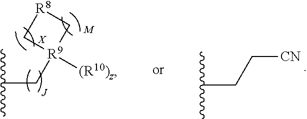

In some embodiments, R²⁰⁰ is a corresponding moiety of a compound or formula provided herein. In some embodiments, R²⁰⁰ is R⁷ or R¹⁷.

In some embodiments, R³⁰⁰ is a corresponding moiety of a compound or formula provided herein. In some embodiments, R³⁰⁰ is R¹⁶, R¹⁸, or

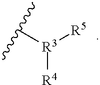

TABLE 1
Selected compounds described herein, and corresponding JAK IC₅₀ (nM).

| Simplified Molecular-Input Line- | | | | |
|---|---|---|---|---|
| Entry String (SMILES)* | JAK1 | JAK2 | JAK3 | TYK2 |
| CC1═NSC(═C1)NC2═NC | <1k | <10 | <1k | <10 |
| (═CC═N2)C3═C[N](CCC#N)N═C3 | | | | |
| CC1═NSC(═C1)NC2═NC(═C | <10 | <10 | <10 | <10 |
| (Cl)C═N2)C3═C[N](CCC#N)N═C3 | | | | |
| CC1═NSC(═C1)NC2═NC(═C | <10 | <10 | <10 | <10 |
| (C)C═N2)C3═C[N](CCC#N)N═C3 | | | | |
| O═C(NC1═NC(═CC═N1)C2═C[N] | <100k | <100k | >50000 | <100k |
| (CCC#N)N═C2)C3CC3 | | | | |
| CC1═C(N═C(NC2═CC═C | <100 | <10 | <100 | <10 |
| (C═C2)N3CCOCC3)N═C1)C4═C[N] | | | | |
| (CCC#N)N═C4 | | | | |
| CC1═C(N═C(NCC═C)N═C1)C2═C[N] | <100k | <100k | <100k | <100k |
| (CCC#N)N═C2 | | | | |
| CC1═C(N═C(N═C1)N2CCCCC2)C3═C[N] | >50000 | >50000 | >50000 | >50000 |
| (CCC#N)N═C3 | | | | |
| CC1═C(N═C(NC2═CC═C(C═C2)C3CN | <100 | <10 | <100 | <10 |
| (C3)C(═O)OC(C)(C)C)N═C1)C4═C[N] | | | | |
| (CCC#N)N═C4 | | | | |
| CC1═NSC(═C1)NC2═NC(═C | <10 | <10 | <10 | <10 |
| (C)C═N2)C3═C[N](CC4(CC4)C#N)N═C3 | | | | |
| CC1═C(N═C(NC2═CC═C | <1k | <100 | <1k | <100 |
| (C═C2F)N3CCOCC3)N═C1)C4═C[N] | | | | |
| (CCC#N)N═C4 | | | | |
| CC1═C(N═C(NCCN2CCCCC2)N═C1)C3═C[N] | >50000 | >50000 | >50000 | >50000 |
| (CCC#N)N═C3 | | | | |
| CC1═C(N═C(NCCN2CCOCC2)N═C1)C3═C[N] | <100k | <100k | >50000 | <100k |
| (CCC#N)N═C3 | | | | |
| CC1═C(N═C(NC2CCOCC2)N═C1)C3═C[N] | <100k | <100k | >50000 | >50000 |
| (CCC#N)N═C3 | | | | |
| ClC1═C(N═C(NC2═CC═C | <10 | <10 | <10 | <10 |
| (C═C2)N3CCOCC3)N═C1)C4═C[N] | | | | |
| (CCC#N)N═C4 | | | | |
| CC1═C(N═C(NC2═CC═C(N3CCOCC3)C | <100 | <10 | <100 | <10 |
| (═C2)F)N═C1)C4═C[N](CCC#N)N═C4 | | | | |
| CC1═C(N═C(NC2═CC═C | <100 | <10 | <100 | <10 |
| (N═C2)N3CCOCC3)N═C1)C4═C[N] | | | | |
| (CCC#N)N═C4 | | | | |
| FC1═CC(═CC═C1N2CCOCC2)NC3═NC(═C | <10 | <10 | <10 | <10 |
| (Cl)C═N3)C4═C[N](CCC#N)N═C4 | | | | |
| CC1═C(N═C(NC2═CC═C | <100 | <10 | <100 | <10 |
| (C═C2)C3CNC3)N═C1)C4═C[N] | | | | |
| (CCC#N)N═C4 | | | | |
| CN(C)CC1═CC═CC(═C1)NC2═NC | <100 | <10 | <100 | <10 |
| (═C(C)C═N2)C3═C[N](CCC#N)N═C3 | | | | |
| FC1═CC(═CC═C1NC2═NC(═C | <1k | <10 | <100 | <100 |
| (Cl)C═N2)C3═C[N] | | | | |
| (CCC#N)N═C3)N4CCOCC4 | | | | |
| CN(C)CC1═CC═C(NC2═NC(═C | <100 | <10 | <100 | <10 |
| (C)C═N2)C3═C[N](CCC#N)N═C3)C═C1 | | | | |
| ClC1═C(N═C(NC2═CC═C | <100 | <10 | <100 | <10 |
| (N═C2)N3CCOCC3)N═C1)C4═C[N] | | | | |
| (CCC#N)N═C4 | | | | |
| CC1═C(N═C(NC2═CC═C | <100 | <10 | <100 | <10 |
| (CCO)C═C2)N═C1)C3═C[N] | | | | |
| (CCC#N)N═C3 | | | | |
| COC(═O)C1═C(C═CC(═C1)NC2═NC(═C | <10 | <10 | <10 | <10 |
| (C)C═N2)C3═C[N] | | | | |
| (CCC#N)N═C3)N4CCOCC4 | | | | |
| CC1═C(N═C(NC2═CC═C(N3CCOCC3)C | <1k | <10 | <1k | <100 |
| (═C2)C(O)═O)N═C1)C4═C[N] | | | | |
| (CCC#N)N═C4 | | | | |
| OCCC1═CC═C(NC2═NC(═C | <100 | <10 | <100 | <10 |
| (Cl)C═N2)C3═C[N](CCC#N)N═C3)C═C1 | | | | |
| ClC1═C(N═C(NC2═CSC═C2)N═C1)C3═C[N] | <100k | <100k | <100k | <100k |
| (CCC#N)N═C3 | | | | |
| CC(C)(C)OC(═O)N1CCN(CC1)C2═CC═C | <100 | <10 | <10 | <10 |
| (NC3═NC(═C(Cl)C═N3)C4═C[N] | | | | |
| (CCC#N)N═C4)C═C2 | | | | |
| CC1═C(N═C(NCCCN2CCOCC2)N═C1)C3═C[N] | >50000 | <100k | >50000 | <100k |
| (CCC#N)N═C3 | | | | |
| CC1═C(N═C(NCCO)N═C1)C2═C[N] | <100k | <100k | <100k | <100k |
| (CCC#N)N═C2 | | | | |
| CC1═NSC(═C1)NC2═NC(═C | <10 | <10 | <10 | <10 |
| (Cl)C═N2)C3═C[N](CC4(CC4)C#N)N═C3 | | | | |
| ClC1═C(N═C(NC2═CC═C | <100 | <10 | <10 | <10 |
| (C═C2)N3CC[S](═O) | | | | |
| (═O)CC3)N═C1)C4═C[N](CCC#N)N═C4 | | | | |
| C[N]1C═CC(═N1)NC2═NC(═C | <1k | <10 | <1k | <1k |
| (Cl)C═N2)C3═C[N](CCC#N)N═C3 | | | | |
| COC(═O)C1═CC═CC(═C1)NC2═NC | <100 | <10 | <10 | <100 |
| (═C(Cl)C═N2)C3═C[N](CCC#N)N═C3 | | | | |
| COC(═O)C1═CC═C(NC2═NC(═C | <100 | <10 | <100 | <10 |
| (Cl)C═N2)C3═C[N](CCC#N)N═C3)C═C1 | | | | |
| CN(C)CC1═CC═CC(═C1)NC2═NC | <100 | <10 | <10 | <100 |
| (═C(Cl)C═N2)C3═C[N](CC4(CC4)C#N)N═C3 | | | | |
| COC(═O)C1═CC(═CC═C1N2CCOCC2)NC3═NC | <10 | <10 | <10 | <10 |
| (═C(Cl)C═N3)C4═C[N](CCC#N)N═C4 | | | | |
| O[P](O)(═O)OCCC1═CC═C(NC2═NC(═C | <100 | <10 | <100 | <10 |
| (Cl)C═N2)C3═C[N](CCC#N)N═C3)C═C1 | | | | |
| CC(═O)OCCC1═CC═C(NC2═NC(═C | <100 | <10 | <100 | <10 |
| (Cl)C═N2)C3═C[N](CCC#N)N═C3)C═C1 | | | | |
| ClC1═C(N═C(NC2═CC═C | <100 | <10 | <100 | <100 |
| (C═C2)C3═NN═N[NH]3)N═C1)C4═C[N] | | | | |
| (CCC#N)N═C4 | | | | |
| CC1═NSC(═C1)NC2═NC(═C | <100 | <10 | <100 | <10 |
| (Cl)C═N2)C3═C[N](CC4(CN(C4)C(═O)OC | | | | |
| (C)(C)C)C#N)N═C3 | | | | |
| ClC1═C(N═C(NC2═CC═C | <100k | <100k | <100k | <100k |
| (C═C2)N3CCNCC3)N═C1)C4═C[N] | | | | |
| (CC5(CC5)C#N)N═C4 | | | | |
| OC(═O)C1═CC═CC(═C1)NC2═NC | <1k | <10 | <1k | <1k |
| (═C(Cl)C═N2)C3═C[N](CCC#N)N═C3 | | | | |
| OC(═O)C1═CC═C(NC2═NC(═C | <10 | <10 | <100 | <10 |
| (Cl)C═N2)C3═C[N](CCC#N)N═C3)C═C1 | | | | |
| COC(═O)C1═NC═C(NC2═NC(═C | <1k | <10 | <100 | <100 |
| (Cl)C═N2)C3═C[N](CC4 | | | | |
| (CC4)C#N)N═C3)C═C1 | | | | |
| CN1CCN(CC1)C2═CC═C(NC3═NC(═C | <10 | <10 | <10 | <10 |
| (Cl)C═N3)C4═C[N](CC5 | | | | |
| (CC5)C#N)N═C4)C═C2 | | | | |
| CN1CCN(CC1)C2═NC═C(NC3═NC(═C | <100k | <1k | <100k | <100k |
| (Cl)C═N3)C4═C[N](CC5 | | | | |
| (CC5)C#N)N═C4)C═C2 | | | | |
| OC(═O)C1═NC═C(NC2═NC(═C | <100 | <10 | <1k | <100 |
| (Cl)C═N2)C3═C[N](CC4 | | | | |
| (CC4)C#N)N═C3)C═C1 | | | | |
| CC1═NSC(═C1)NC2═NC(═C | <10 | <10 | <10 | <10 |
| (Cl)C═N2)C3═C[N](CC4(CNC4)C#N)N═C3 | | | | |
| CC1═NSC(═C1)NC2═NC(═C | <1k | <100 | <100 | <1k |
| (Cl)C═N2)C3═C[N](CC4(CNC4)C | | | | |
| (O)═O)N═C3 | | | | |
| CN(C)CC1═CC═CC(═C1)NC2═NC | <100 | <10 | <100 | <100 |
| (═C(Cl)C═N2)C3═C[N] | | | | |
| (CC4═CC═CS4)N═C3 | | | | |
| CN(C)CC1═CC═CC(═C1)NC2═NC | <100k | <100 | <1k | <1k |
| (═CC═N2)C3═C[N] | | | | |
| (CC4═CC═CS4)N═C3 | | | | |
| CC1═NSC(═C1)NC2═NC(═C | <100 | <10 | <10 | <10 |
| (Cl)C═N2)C3═C[N](CC4(CCC4)C#N)N═C3 | | | | |
| CN1CC2═C(C1)C═C(NC3═NC(═C | <10 | <10 | <10 | <10 |
| (Cl)C═N3)C4═C[N](CCC#N)N═C4)C═C2 | | | | |
| CN(C)CC1═CC═CC(═C1)NC2═NC | <100 | <10 | <10 | <100 |
| (═C(Cl)C═N2)C3═C[N](CC4 | | | | |
| (CCC4)C#N)N═C3 | | | | |
| CC1═NSC(═C1)NC2═NC(═C | <10 | <10 | <10 | <10 |
| (Cl)C═N2)C3═C[N](CC4(COC4)C#N)N═C3 | | | | |
| CN(C)CC1═CC═CC(═C1)NC2═NC | <100 | <10 | <100 | <100 |
| (═C(Cl)C═N2)C3═C[N](CC4 | | | | |
| (COC4)C#N)N═C3 | | | | |
| CC1═NSC(═C1)NC2═NC | <1k | <100 | <1k | <1k |
| (═C3CCCC3═N2)C4═C[N](CC5(CC5)C#N)N═C4 | | | | |
| ClC1═C(N═C(NC23CC(C2)C3)N═C1)C4═C[N] | >50000 | >50000 | <100k | >50000 |
| (CC5═CC═CS5)N═C4 | | | | |
| CN(C)CC1═CC(═CC═C1)NC2═NC | <100k | <1k | <100k | <100k |
| (═C3CCCC3═N2)C4═C[N](CCC#N)N═C4 | | | | |
| CC1═NSC(═C1)NC2═NC(═C3CN | >50000 | >50000 | >50000 | >50000 |
| (CC3═N2)C(═O)OC(C)(C)C)C4═C[N] | | | | |
| (CC5(COC5)C#N)N═C4 | | | | |
| CC1═NSC(═C1)NC2═NC(═C | <100 | <10 | <100 | <10 |
| (Cl)C═N2)C3═C[N](CC4═CC═CS4)N═C3 | | | | |
| CC1═NSC(═C1)NC2═NC(═C | <100 | <10 | <100 | <100 |
| (C═N2)C3CC3)C4═C[N](CC5 | | | | |
| (COC5)C#N)N═C4 | | | | |
| CN(C)CC1═CC(═CC═C1)NC2═NC | <100k | <100 | <100k | <100k |
| (═C3CCCC3═N2)C4═C[N](CC5 | | | | |
| (CC5)C#N)N═C4 | | | | |
| CC1═NSC(═C1)NC2═NC(═C | <100 | <10 | <100 | <100 |
| (C═N2)C3CC3)C4═C[N](CC5 | | | | |
| (CCC5)C#N)N═C4 | | | | |
| CC1═NSC(═C1)NC2═NC(═C | <100 | <10 | <100 | <10 |
| (C═N2)C3CC3)C4═C[N](CC5 | | | | |
| (CC5)C#N)N═C4 | | | | |
| ClC1═C(N═C | <100k | <100 | <1k | <1k |
| (NC2═C[N]3N═CC═C3N═C2)N═C1)C4═C[N] | | | | |
| (CC5(CCC5)C#N)N═C4 | | | | |
| CN1/C═C\C(═C/C1═O)NC2═NC(═C | <1k | <10 | <1k | <1k |
| (Cl)C═N2)C3═C[N](CC4(COC4)C#N)N═C3 | | | | |
| CN(C)CC1═CC═CC(═C1)NC2═NC | <1k | <10 | <1k | <1k |
| (═C(C═N2)C3CC3)C4═C[N](CC5 | | | | |
| (COC5)C#N)N═C4 | | | | |
| COC(═O)C1═CC(═CC═C1N2CCOCC2)NC3═NC | <100 | <10 | <10 | <100 |
| (═C(C═N3)C4CC4)C5═C[N](CC6 | | | | |
| (CC6)C#N)N═C5 | | | | |
| C[S](═O)(═O)N(C1═NC(═C | >50000 | <100k | >50000 | >50000 |
| (Cl)C═N1)C2═C[N](CCC#N)N═C2)[S] | | | | |
| (C)(═O)═0 | | | | |
| C[S](═O)(═O)NC1═NC(═C | <100k | <100k | <100k | <100k |
| (Cl)C═N1)C2═C[N](CCC#N)N═C2 | | | | |
| CN(C)CC1═CC═CC(═C1)NC2═NC | <1k | <10 | <100 | <1k |
| (═C(Cl)C═N2)C3═C[N](CCC4COC4)N═C3 | | | | |
| CC1═NSC(═C1)NC2═NC(═C | <100 | <10 | <100 | <100 |
| (Cl)C═N2)C3═C[N](CCC4COC4)N═C3 | | | | |
| FC1CN(C1)CC2═CC═CC(═C2)NC3═NC(═C | <100 | <10 | <10 | <10 |
| (Cl)C═N3)C4═C[N](CCC#N)N═C4 | | | | |
| CN(C)CC1═CC═CC(═C1)NC2═NC | <1k | <10 | <1k | <1k |
| (═C(C═N2)C3CC3)C4═C[N](CC5 | | | | |
| (CCC5)C#N)N═C4 | | | | |
| CC1═NSC(═C1)NC2═NC(═C | <100 | <10 | <10 | <10 |
| (Cl)C═N2)C3═C[N](CC4(F)CC4)N═C3 | | | | |
| CC1═NSC(═C1)NC2═NC(═C | <100 | <10 | <100 | <10 |
| (Cl)C═N2)C3═C[N](CC4═CSC═C4)N═C3 | | | | |
| CN1CCN(CC1)C2═CC═C(NC3═NC(═C(C([2H]) | <100 | <10 | <10 | <10 |
| ([2H])[2H])C(═N3)[2H])C4═C[N] | | | | |
| (CC5(CC5)C#N)N═C4)C═C2 | | | | |
| CC(C)(C)OC(═O)N1CCC(NC2═NC(═C | >50000 | >50000 | >50000 | >50000 |
| (Cl)C═N2)C3═C[N](CCC#N)N═C3)C | | | | |
| (F)(F)C1 | | | | |
| NC(═O)CC[N]1C═C(C═N1)C2═C | >50000 | <100k | >50000 | >50000 |
| (Cl)C═NC(═N2)NC3CCNCC3(F)F | | | | |
| FC1(F)CNCCC1NC2═NC(═C | >50000 | <100k | >50000 | >50000 |
| (Cl)C═N2)C3═C[N](CCC#N)N═C3 | | | | |
| ClC1═C(N═C(NC2═CC(═CC═C2)CN3CCC3)N═C1) | <100 | <10 | <10 | <10 |
| C4═C[N](CCC#N)N═C4 | | | | |
| CC1═C(N═C(NCC2═CC═CC | <100k | <100k | 50000 | <100k |
| (═C2)Cl)N═C1)C3═C[N](CCC#N)N═C3 | | | | |
| CC1═NSC(═C1)NC2═NC(═C | <10 | <10 | <10 | <10 |
| (Cl)C═N2)C3═C[N](CC4(CF)CC4)N═C3 | | | | |
| NC(═O)C1═CC═C(NC2═NC(═C | <10 | <10 | <10 | <10 |
| (Cl)C═N2)C3═C[N](CC4 | | | | |
| (CC4)C#N)N═C3)C═C1 | | | | |
| NC(═O)C1═CC═CC(═C1)NC2═NC | <100 | <10 | <10 | <100 |
| (═C(Cl)C═N2)C3═C[N](CC4 | | | | |
| (CC4)C#N)N═C3 | | | | |
| CC1═NSC(═C1)NC2═NC(═C(C([2H]) | <10 | <10 | <10 | <10 |
| ([2H])[2H])C(═N2)[2H])C3═C[N] | | | | |
| (CC4(CC4)C#N)N═C3 | | | | |
| C(C1═C(N═C(NC2═CC═C(C═C2)N3CCOCC3) | <100 | <10 | <10 | <10 |
| N═C1[2H])C4═C[N](CC5(CC5)C#N)N═C4) | | | | |
| ([2H])([2H])[2H] | | | | |
| CC1═NSC(═C1)NC2═NC(═C(Cl)C═N2)C3═C[N] | <1k | <10 | <100 | <100 |
| (N═C3)C4CCOCC4 | | | | |
| CN(C)CC1═CC(═CC═C1)NC2═NC | <1k | <10 | <100 | <1k |
| (═C(Cl)C═N2)C3═C[N](N═C3)C4CCOCC4 | | | | |
| CC1═NSC(═C1)NC2═NC(═C | <100 | <10 | <100 | <10 |
| (Cl)C═N2)C3═C[N](CCC(F)(F)F)N═C3 | | | | |
| CN(C)CC1═CC(═CC═C1)NC2═NC | <100 | <10 | <100 | <100 |
| (═C(Cl)C═N2)C3═C[N](CCC(F)(F)F)N═C3 | | | | |
| CN1CCN(CC1)C2═CC═C(NC3═NC | <100 | <10 | <10 | <10 |
| (═C(Cl)C═N3)C4═C[N](CC5 | | | | |
| (F)CC5)N═C4)C═C2 | | | | |
| CN(C)CC1═CC═CC(═C1)NC2═NC | <100 | <10 | <100 | <10 |
| (═C(Cl)C═N2)C3═C[N](CCC#N)N═C3 | | | | |
| CC1═NSC(═C1)NC2═NC(═C | <100 | <10 | <10 | <10 |
| (Cl)C═N2)C3═C[N](CCC4CC4)N═C3 | | | | |
| CN1CCN(CC1)C2═CC═C(NC3═NC | <100 | <10 | <10 | <100 |
| (═C(Cl)C═N3)C4═C[N] | | | | |
| (CCC5CC5)N═C4)C═C2 | | | | |
| CC1═CC═C(C═C1)[S](═O)(═O)NC2═NC | 50000 | <100k | 50000 | <100k |
| (═C(Cl)C═N2)C3═C[N](CCC#N)N═C3 | | | | |
| CN1CCN(CC1)C2═CC═C(NC3═NC | <100 | <10 | <10 | <10 |
| (═C(Cl)C═N3)C4═C[N](CC5 | | | | |
| (CF)CC5)N═C4)C═C2 | | | | |
| CC1═NSC(═C1)NC2═NC(═C | <100 | <10 | <100 | <100 |
| (Cl)C═N2)C3═C[N](CC4═NC═C | | | | |
| (F)C═N4)N═C3 | | | | |
| CC1═NSC(═C1)NC2═NC(═C | <10 | <10 | <10 | <10 |
| (Cl)C═N2)C3═C[N](CC4(Cl)CC4)N═C3 | | | | |
| C[N]1C═C(NC2═NC(═C | <100 | <10 | <10 | <100 |
| (Cl)C═N2)C3═C[N](CCC4CC4)N═C3)C═N1 | | | | |
| CN1CCN(CC1)C2═CC═C(NC3═NC | <10 | <10 | <10 | <10 |
| (═C(Cl)C═N3)C4═C[N](CC5 | | | | |
| (Cl)CC5)N═C4)C═C2 | | | | |
| CN(C)CC1═CC═CC(═C1)NC2═NC(═C | <100 | <10 | <10 | <100 |
| (Cl)C═N2)C3═C[N](CC4(CF)CC4)N═C3 | | | | |
| CCOC(═O)C[N]1C═C(C═N1)C2═C | <1k | <10 | <100 | <1k |
| (Cl)C═NC(═N2)NC3═CC(═CC═C3)CN(C)C | | | | |
| CCOC(═O)C[N]1C═C(C═N1)C2═C | <100 | <10 | <100 | <100 |
| (Cl)C═NC(═N2)NC3═CC(═NS3)C | | | | |
| ClC1═C(N═C(N═C1)N(C(═O)C2═CC═CC═C2)C | 50000 | <100k | 50000 | 50000 |
| (═O)C3═CC═CC═C3)C4═C[N] | | | | |
| (CCC#N)N═C4 | | | | |
| CC1═NSC(═C1)NC2═NC(═C(C([2H]) | <100 | <10 | <10 | <10 |
| ([2H])[2H])C(═N2)[2H])C3═C[N] | | | | |
| (CC4(F)CC4)N═C3 | | | | |
| CN1CCN(CC1)C2═CC═C(NC3═NC(═C(C | <100 | <10 | <10 | <10 |
| ([2H])([2H])[2H])C(═N3)[2H])C4═C[N] | | | | |
| (CC5(F)CC5)N═C4)C═C2 | | | | |
| CC1═NSC(═C1)NC2═NC(═C | <100 | <10 | <100 | <100 |
| (Cl)C═N2)C3═C[N](CC4═NC═C | | | | |
| (F)C═C4)N═C3 | | | | |
| CC1═NSC(═C1)NC2═NC(═C | <10 | <10 | <10 | <10 |
| (Cl)C═N2)C3═C[N](CC4(CC4)C(F)F)N═C3 | | | | |
| FCC1(CC1)C[N]2C═C(C═N2)C3═C | <10 | <10 | <10 | <10 |
| (Cl)C═NC(═N3)NC4═CC5═C(C═C4)C | | | | |
| (═O)NC5 | | | | |
| CC1═NSC(═C1)NC2═NC(═C | <10 | <10 | <10 | <10 |
| (C)C═N2)C3═C[N](CC4(Cl)CC4)N═C3 | | | | |
| CC(C)(C)OC(═O)NCC1═CC═CC | 156.6 | <100 | <10 | <100 |
| (═C1)NC2═NC(═C(Cl)C═N2)C3═C[N] | | | | |
| (CC4(CF)CC4)N═C3 | | | | |
| NCC1═CC═CC(═C1)NC2═NC(═C | <100 | <10 | <10 | <100 |
| (Cl)C═N2)C3═C[N](CC4(CF)CC4)N═C3 | | | | |
| CC1═NSC(═C1)NC2═NC(═C | <100 | <10 | <100 | <10 |
| (Cl)C═N2)C3═C[N](CC4(F)COC4)N═C3 | | | | |
| CN1CCN(CC1)C2═CC═C(NC3═NC(═C | <100 | <10 | <10 | <100 |
| (Cl)C═N3)C4═C[N](CC5(F)COC5)N═C4)C═C2 | | | | |
| CC1═NSC(═C1)NC2═NC(═C | <10 | <10 | <10 | <10 |
| (Cl)C═N2)C3═C[N](CC4(CF)COC4)N═C3 | | | | |
| CN1CCN(CC1)C2═CC═C(NC3═NC(═C | <100 | <10 | <10 | <10 |
| (Cl)C═N3)C4═C[N](CC5 | | | | |
| (CF)COC5)N═C4)C═C2 | | | | |
| CN1CCN(CC1)C2═CC═C(NC3═NC(═C | <100 | <10 | <10 | <10 |
| (Cl)C═N3)C4═C[N](CC5(COC5)C | | | | |
| (F)F)N═C4)C═C2 | | | | |
| CC1═NSC(═C1)NC2═NC(═C | <10 | <10 | <10 | <10 |
| (Cl)C═N2)C3═C[N](CC4(COC4)C(F)F)N═C3 | | | | |
| CN1CCN(CC1)C2═CC═C(NC3═NC | <100 | <10 | <100 | <100 |
| (═C(C)C═N3)C4═C[N](CC5 | | | | |
| (CC5)C#N)N═C4)C═C2 | | | | |
| CN(C)CC1═CC═C(NC2═NC(═C | <100k | <100 | <1k | <100k |
| (Cl)C═N2)C3═C[N](CC4(CF)CC4)N═C3)N═C1 | | | | |
| CN(C)CC1═CC═NC(═C1)NC2═NC(═C | <1k | <100 | <1k | <100k |
| (Cl)C═N2)C3═C[N](CC4(CF)CC4)N═C3 | | | | |
| CC(C)(C)OC(═O)[N]1C═CC | <100k | <100 | <1k | <100k |
| (═N1)NC2═NC(═C(Cl)C═N2)C3═C[N] | | | | |
| (CC4(CF)CC4)N═C3 | | | | |
| CN1CCN(CC1)C2═CC═C(NC3═NC | <10 | <10 | <10 | <10 |
| (═C(C)C═N3)C4═C[N](CC5 | | | | |
| (Cl)CC5)N═C4)C═C2 | | | | |
| CN1CCN(CC1)C2═NC═C(NC3═NC | <100 | <10 | <100 | <100 |
| (═C(Cl)C═N3)C4═C[N](CC5 | | | | |
| (CF)COC5)N═C4)C═C2 | | | | |
| CC1═CSC(═N1)NC2═NC(═C | <1k | <100 | <1k | <1k |
| (Cl)C═N2)C3═C[N](CC4(CF)COC4)N═C3 | | | | |
| CN(C)CC1═CC═CC(═C1)NC2═NC(═C | <10 | <10 | <10 | <10 |
| (C)C═N2)C3═C[N](CC4(Cl)CC4)N═C3 | | | | |
| FCC1(CC1)C[N]2C═C(C═N2)C3═C | <1k | <10 | <100 | <1k |
| (Cl)C═NC(═N3)NC4═N[NH]C═C4 | | | | |
| CN1CC2═C(C1)C═C(NC3═NC(═C | <10 | <10 | <10 | <10 |
| (Cl)C═N3)C4═C[N](CC5(CF)CC5)N═C4)C═C2 | | | | |
| CC1═NSC(═C1)NC2═NC(═C | <100 | <10 | <100 | <10 |
| (C)C═N2)C3═C[N](CCCO)N═C3 | | | | |
| CC1═NSC(═C1)NC2═NC(═C(C([2H]) | <10 | <10 | <10 | <10 |
| ([2H])[2H])C(═N2)[2H])C3═C[N](CC4 | | | | |
| (Cl)CC4)N═C3 | | | | |
| CC1═NC═C(NC2═NC(═C | <100 | <10 | <100 | <100 |
| (Cl)C═N2)C3═C[N](CC4(CF)COC4)N═C3)S1 | | | | |
| CN(C(═O)OC(C)(C)C)C1(CC1)C[N]2C═C | <1k | <100 | <100 | <1k |
| (C═N2)C3═C(Cl)C═NC(═N3)NC4═CC(═NS4)C | | | | |
| CNC1(CC1)C[N]2C═C(C═N2)C3═C | <100 | <10 | <10 | <100 |
| (Cl)C═NC(═N3)NC4═CC(═NS4)C | | | | |
| FCC1(COC1)C[N]2C═C(C═N2)C3═C | <1k | <100 | <1k | <1k |
| (Cl)C═NC(═N3)NC4═CC═NC═C4 | | | | |
| FCC1(COC1)C[N]2C═C(C═N2)C3═C | <1k | <10 | <100 | <1k |
| (Cl)C═NC(═N3)NC4═CN═CC═C4 | | | | |
| FC(F)C1(COC1)C[N]2C═C(C═N2)C3═C | <1k | <10 | <100 | <1k |
| (Cl)C═NC(═N3)NC4═CC5═C(C═C4)C═NO5 | | | | |
| CC1═NOC(═C1)NC2═NC(═C | <100 | <10 | <1k | <100 |
| (Cl)C═N2)C3═C[N](CCC#N)N═C3 | | | | |
| CC1═CC(═NO1)NC2═NC(═C | <1k | <100 | <1k | <100 |
| (Cl)C═N2)C3═C[N](CCC#N)N═C3 | | | | |
| FC(F)(F)C1═NOC(═C1)NC2═NC(═C | >50000 | <1k | >50000 | >50000 |
| (Cl)C═N2)C3═C[N](CCC#N)N═C3 | | | | |
| CC1═C(N═C(NC2═CC3═C(C═C2)C | <10 | <10 | <10 | <10 |
| (═O)NC3)N═C1)C4═C[N](CC5 | | | | |
| (Cl)CC5)N═C4 | | | | |
| CN1C(═O)C2═C(C═C(NC3═NC(═C | <1k | <10 | <100 | <100 |
| (C)C═N3)C4═C[N](CC5 | | | | |
| (Cl)CC5)N═C4)C═C2)C1═0 | | | | |
| CN1CC2═C(C1)C═C(NC3═NC(═C | <100 | <10 | <100 | <100 |
| (C)C═N3)C4═C[N](CC5 | | | | |
| (Cl)CC5)N═C4)C═C2 | | | | |
| CC1═C(N═C(NC2═CC═C(C═C2)[S] | <100 | <10 | <10 | <10 |
| (C)(═O)═O)N═C1)C3═C[N](CC4 | | | | |
| (Cl)CC4)N═C3 | | | | |
| CC1═C(N═C(NC2═CC═C(C(═C2)F)[S] | <100 | <10 | <100 | <10 |
| (C)(═O)═O)N═C1)C3═C[N](CC4 | | | | |
| (Cl)CC4)N═C3 | | | | |
| CN(C)CC1═CC(═CC═C1)NC2═NC(═C | <1k | <100 | <100 | <1k |
| (Cl)C═N2)C3═C[N](CC4(CO)CN | | | | |
| (C4)C(═O)OC(C)(C)C)N═C3 | | | | |
| CNC(═O)C1═CC═CC(═C1)NC2═NC | <1k | <10 | <100 | <1k |
| (═C(Cl)C═N2)C3═C[N](CC4 | | | | |
| (CC4)NC)N═C3 | | | | |
| CN(C)CC1(CC1)C[N]2C═C(C═N2)C3═C | <100 | <10 | <100 | <100 |
| (C)C═NC(═N3)NC4═CC(═NS4)C | | | | |
| CC1═CC═C(NC2═NC(═C(C)C═N2)C3═C[N] | <100 | <100 | <100 | <100 |
| (CC4(Cl)CC4)N═C3)C═C1[S](C) | | | | |
| (═O)═0 | | | | |
| CN(C)CC1═CC(═CC═C1)NC2═NC(═C | <1k | <100 | <100 | <1k |
| (Cl)C═N2)C3═C[N](CC4(CO)CNC4)N═C3 | | | | |
| CC(C)(C)OC(═O)N1CC(CO)(C1)C[N]2C═C | <1k | <100 | <100 | <1k |
| (C═N2)C3═C(Cl)C═NC(═N3)NC4═CC═C | | | | |
| (C═C4)C(N)═O | | | | |
| NC(═O)C1═CC═C(NC2═NC(═C | <100 | <100 | <100 | <1k |
| (Cl)C═N2)C3═C[N](CC4(CO)CNC4)N═C3)C═C1 | | | | |
| CC(C)(C)OC(═O)N1CC(CF)(C1)C[N]2C═C | <100 | <100 | <100 | <100 |
| (C═N2)C3═C(Cl)C═NC(═N3)NC4═CC═C | | | | |
| (C═C4)C(N)═O | | | | |
| COC1═C(N═C(NC2═CC | <10 | <10 | <10 | <10 |
| (═NS2)C)N═C1)C3═C[N](CC4(Cl)CC4)N═C3 | | | | |
| CC1═NSC(═C1)NC2═NC(═C | <10 | <10 | <10 | <10 |
| (C)C═N2)C3═C[N](CC4(CF)CC4)N═C3 | | | | |
| CC1═C(N═C(NC2═CC═C(CO)C | <100 | <10 | <100 | <10 |
| (═C2)CO)N═C1)C3═C[N](CC4(CF)CC4)N═C3 | | | | |
| CN(C)CC1═CC(═CC═C1)NC2═NC | <100 | <10 | <100 | <100 |
| (═C(Cl)C═N2)C3═C[N](CC4(CN | | | | |
| (C4)C(═O)OC(C)(C)C)C#N)N═C3 | | | | |
| CN(C)CC1═CC(═CC═C1)NC2═NC | <1k | <10 | <100 | <100 |
| (═C(Cl)C═N2)C3═C[N](CC4(CF)CN | | | | |
| (C4)C(═O)OC(C)(C)C)N═C3 | | | | |
| CC1═NSC(═C1)NC2═NC(═C(Cl)C═N2)C3═C[N] | <100 | <10 | <100 | <100 |
| (CC4(CF)CN(C4)C(═O)OC(C)(C)C)N═C3 | | | | |
| CCC1═C(N═C(NC2═CC | <100 | <10 | <10 | <100 |
| (═NS2)C)N═C1)C3═C[N](CC4(Cl)CC4)N═C3 | | | | |
| CC1═NSC(═C1)NC2═NC(═C | <100 | <10 | <100 | <100 |
| (C═N2)C3CC3)C4═C[N](CC5(Cl)CC5)N═C4 | | | | |
| CC1═C(N═C(NC2═CC═CC═N2)N═C1)C3═C[N] | <100k | <1k | <100k | <100k |
| (CC4(Cl)CC4)N═C3 | | | | |
| FCC1(COC1)C[N]2C═C(C═N2)C3═C | <100k | <1k | <100k | <100k |
| (Cl)C═NC(═N3)NC4═CC═CC═N4 | | | | |
| NC(═O)C1═CC═C(NC2═NC(═C | <100 | <10 | <10 | <100 |
| (Cl)C═N2)C3═C[N](CC4(CF)CNC4)N═C3)C═C1 | | | | |
| CN(C)CC1═CC(═CC═C1)NC2═NC(═C | <1k | <10 | <10 | <1k |
| (Cl)C═N2)C3═C[N](CC4(CF)CNC4)N═C3 | | | | |
| CC1═NSC(═C1)NC2═NC(═C | <1k | <100 | <100 | <100 |
| (Cl)C═N2)C3═C[N](CC4(CO)CN(C4)C | | | | |
| (═O)OC(C)(C)C)N═C3 | | | | |
| CC1═NSC(═C1)NC2═NC(═C | <100 | <10 | <10 | <10 |
| (Cl)C═N2)C3═C[N](CC4(CF)CNC4)N═C3 | | | | |
| CC1═NSC(═C1)NC2═NC(═C | <100 | <10 | <10 | <100 |
| (Cl)C═N2)C3═C[N](CC4(CO)CNC4)N═C3 | | | | |
| CC[S](═O)(═O)N1CC(CF)(C[N]2C═C | <100 | <10 | <100 | <100 |
| (C═N2)C3═C(Cl)C═NC(═N3)NC4═CC | | | | |
| (═NS4)C)C1 | | | | |
| CN(C)CC1═CC(═CC═C1)NC2═NC(═C | <100 | <10 | <10 | <100 |
| (Cl)C═N2)C3═C[N](CC4(CNC4)C#N)N═C3 | | | | |
| CN1CC2═CC═CC(═C2C1)NC3═NC(═C | <100k | <1k | <100k | 50000 |
| (C)C═N3)C4═C[N](CC5(Cl)CC5)N═C4 | | | | |
| CC1═C(N═C(NC2═C3CNC | <100k | <100 | <1k | <100k |
| (═O)C3═CC═C2)N═C1)C4═C[N] | | | | |
| (CC5(Cl)CC5)N═C4 | | | | |
| COC1═C(N═C(NC2═CC | <100 | <10 | <10 | <100 |
| (═NS2)C)N═C1)C3═C[N](CC4(CF)CC4)N═C3 | | | | |
| CC1═NSC(═C1)NC2═NC(═C | <1k | <100 | <100k | <1k |
| (C)C═N2)C3═C[N](N═C3)C4CN(C4)C | | | | |
| (═O)OC(C)(C)C | | | | |
| CC1═NSC(═C1)NC2═NC(═C | <100 | <10 | <10 | <10 |
| (F)C═N2)C3═C[N](CC4(Cl)CC4)N═C3 | | | | |
| CC1═NSC(═C1)NC2═NC(═C | <1k | <1k | <1k | <1k |
| (C)C═N2)C3═C[N](N═C3)C4CNC4 | | | | |
| CC1═NSC(═C1)NC2═NC(═C | <10 | <10 | <100 | <10 |
| (C)C═N2)C3═C[N](N═C3)C4CN(CC#N)C4 | | | | |
| CC1═NSC(═C1)NC2═NC(═C | <100 | <100 | <1k | <100 |
| (C)C═N2)C3═C[N](N═C3)C4CN(CF)C4 | | | | |
| CC1═C(N═C(NC(═O)NC2═CC═C | >50000 | >50000 | >50000 | >50000 |
| (Cl)C═C2)N═C1)C3═C[N](CC4(CF)CC4)N═C3 | | | | |
| CC(C)(C)OC(═O)N1CC(C1)(C[N]2C═C | <100 | <10 | <100 | <100 |
| (C═N2)C3═C(Cl)C═NC(═N3)NC4═CC═C | | | | |
| (C═C4)C(N)═O)C#N | | | | |
| CC1═C(N═C(NC(═O)OC2═CC═C | <100k | <1k | <100k | <100k |
| (Cl)C═C2)N═C1)C3═C[N](CC4 | | | | |
| (CF)CC4)N═C3 | | | | |
| CN1CCN(CC1)C2═CC═C(NC3═NC(═C | <100 | <10 | <10 | <10 |
| (C)C═N3)C4═C[N](CC5(CF)CC5)N═C4)C═C2 | | | | |
| NC(═O)C1═CC═C(NC2═NC(═C | <10 | <10 | <10 | <10 |
| (Cl)C═N2)C3═C[N](CC4 | | | | |
| (CNC4)C#N)N═C3)C═C1 | | | | |
| CC1═NSC(═C1)NC2═NC(═C | <100 | <10 | <100 | <100 |
| (C)C═N2)C3═C[N](CC45CC(CO4) | | | | |
| (C5)C#N)N═C3 | | | | |
| CC1═NSC(═C1)NC2═NC(═C | <10 | <10 | <10 | <10 |
| (C)C═N2)C3═C[N](CC4(CCC4)C#N)N═C3 | | | | |
| CC1═NSC(═C1)NC2═NC(═C | <10 | <10 | <10 | <10 |
| (Cl)C═N2)C3═C[N](CC4(CN | | | | |
| (CC#N)C4)C#N)N═C3 | | | | |
| N2)C3═C(Cl)C═NC(═N3)NC4═CC | <10 | <10 | <10 | <10 |
| (═NS4)C)(C1)C#N | | | | |
| CN1CCN(CC1)C2═CC═C(NC3═NC | <100 | <10 | <100 | <100 |
| (═C(C)C═N3)C4═C[N](CC5 | | | | |
| (CF)COC5)N═C4)C═C2 | | | | |
| CC1═C(N═C(NC2═CC═NS2)N═C1)C3═C[N] | <10 | <10 | <10 | <10 |
| (CC4(CF)CC4)N═C3 | | | | |
| CC[S](═O)(═O)N1CC(CO) | <1k | <1k | <100k | <100k |
| (C[N]2C═C(C═N2)C3═C(Cl)C═NC | | | | |
| (═N3)NC4═CC(═NS4)C)C1 | | | | |
| CN1CCN(CC1)C2═CC═C(NC3═NC═C | <100 | <10 | <10 | <10 |
| (C)C(═N3)C4═C[N](CC5 | | | | |
| (CCC5)C#N)N═C4)C═C2 | | | | |
| CC1═NSC(═C1)NC2═NC═C(C)C | <10 | <10 | <10 | <10 |
| (═N2)C3═C[N](CC4(CF)CCC4)N═C3 | | | | |
| CN1CCN(CC1)C2═CC═C(NC3═NC═C | <100 | <10 | <10 | <10 |
| (C)C(═N3)C4═C[N](CC5 | | | | |
| (CF)CCC5)N═C4)C═C2 | | | | |
| CN1CC2═CC═C(NC3═NC(═C | <10 | <10 | <10 | <10 |
| (C)C═N3)C4═C[N](CC5 | | | | |
| (CF)CC5)N═C4)C═C2C1 | | | | |
| CC1═NSC(═C1)NC2═NC(═C | <100 | <100 | <100 | <100 |
| (C)C═N2)C3═C[N](CCN4CCC4)N═C3 | | | | |
| CC1═C(N═C(NC2═CC═C | <1k | <100 | <100 | <1k |
| (C═C2)N3CCOCC3)N═C1)C4═C[N] | | | | |
| (CCN5CCC5)N═C4 | | | | |
| CC1═CN═C(NC2═CC═C | <100 | <10 | <10 | <10 |
| (C═C2)N3CCOCC3)N═C1C4═C[N] | | | | |
| (CC5(CF)CC5)N═C4 | | | | |
| CC1═C(N═C(NC2═CC═NS2)N═C1)C3═C[N] | <100 | <10 | <100 | <10 |
| (CC4(CF)CCC4)N═C3 | | | | |
| CC1═C(N═C(NC2═CC═NS2)N═C1)C3═C[N] | <10 | <10 | <10 | <10 |
| (CC4(Cl)CC4)N═C3 | | | | |
| CC1═C(N═C(NC2═CC(═NS2)C | <100 | <10 | <100 | <100 |
| (O)═O)N═C1)C3═C[N](CC4 | | | | |
| (CF)CC4)N═C3 | | | | |
| CC1═C(N═C(NC2═CC═NS2)N═C1)C3═C[N] | <100 | <10 | <100 | <100 |
| (CCCO)N═C3 | | | | |
| CC1═NSC(═C1)NC2═NC(═C | <10 | <10 | <10 | <10 |
| (C)C═N2)C3═C[N](CC4(CF)COC4)N═C3 | | | | |
| CC1═C(N═C(NC2═CC═NS2)N═C1)C3═C[N] | <100 | <10 | <100 | <100 |
| (CC4(CF)COC4)N═C3 | | | | |
| CC1═C(N═C(NC2═CC(═C | <100 | <10 | <10 | <10 |
| (CO)C═C2)CO)N═C1)C3═C[N] | | | | |
| (CC4(CF)CCC4)N═C3 | | | | |
| CC1═NSC(═C1)NC2═NC(═C | <10 | <10 | <10 | <10 |
| (C)C═N2)C3═C[N](CCC(F)F)N═C3 | | | | |
| CC1═NSC(═C1)NC2═NC(═C | <10 | <10 | <10 | <10 |
| (C)C═N2)C3═C[N](CCCF)N═C3 | | | | |
| CC1═NSC(═C1)NC2═NC(═C | <1k | <100 | <1k | <1k |
| (C)C═N2)C3═C[N](CC4CCN(C4)C | | | | |
| (═O)OC(C)(C)C)N═C3 | | | | |
| CC1═NSC(═C1)NC2═NC(═C | <10 | <10 | <100 | <10 |
| (C)C═N2)C3═C[N](CC(F)F)N═C3 | | | | |
| CN(C)CCOC1═CC═C(NC2═NC(═C | <100k | <1k | <1k | <100k |
| (C)C═N2)C3═C[N](CC4 | | | | |
| (CF)CCC4)N═C3)N═C1 | | | | |
| CC1═NC═C(NC2═NC(═C | <100 | <10 | <100 | <100 |
| (C)C═N2)C3═C[N](CC4(CF)CCC4)N═C3)S1 | | | | |
| CC1═CN═CC(═C1)NC2═NC(═C | <100 | <10 | <100 | <100 |
| (C)C═N2)C3═C[N](CC4(CF)CCC4)N═C3 | | | | |
| CN(C)CCOC1═CC(═CC═N1)NC2═NC | <100 | <10 | <100 | <100 |
| (═C(C)C═N2)C3═C[N](CC4 | | | | |
| (CF)CCC4)N═C3 | | | | |
| CC1═C(N═C(NC2═CN═C | <1k | <10 | <100 | <1k |
| (N═C2)N3CCOCC3)N═C1)C4═C[N] | | | | |
| (CC5(CF)CC5)N═C4 | | | | |
| COC1═C(N═C(NC2═CC═C | <100 | <10 | <10 | <100 |
| (C═C2)N3CCN(C)CC3)N═C1)C4═C[N](CC5 | | | | |
| (Cl)CC5)N═C4 | | | | |
| CC1═C(N═C(NC2═CC═C(CNC(═O)OC | <100 | <10 | <100 | <100 |
| (C)(C)C)C═C2)N═C1)C3═C[N](CC4 | | | | |
| (CF)CC4)N═C3 | | | | |
| CC1═NC═CC(═C1)NC2═NC(═C | <1k | <10 | <100 | <100 |
| (C)C═N2)C3═C[N](CC4(CF)CCC4)N═C3 | | | | |
| CC1═NSC(═C1)NC2═NC(═C | <100 | <10 | <100 | <10 |
| (C)C═N2)C3═C[N](CC4(CCCC4)C#N)N═C3 | | | | |
| CC1═C(N═C(NC2═CC(═NS2)CO)N═C1)C3═C[N] | <10 | <10 | <10 | <10 |
| (CC4(CF)CC4)N═C3 | | | | |
| CC1═C(N═C(NC2═CC═C | <100 | <10 | <100 | <100 |
| (CN)C═C2)N═C1)C3═C[N](CC4 | | | | |
| (CF)CC4)N═C3 | | | | |
| C[C@H](NC(═O)OC(C)(C)C)C | <100 | <10 | <100 | <100 |
| (═O)NCC1═CC═C(NC2═NC(═C | | | | |
| (C)C═N2)C3═C[N](CC4(CF)CC4)N═C3)C═C1 | | | | |
| CN1CCN(CC1)C2═CC═C(NC3═NC(═C | <1k | <10 | <100 | <100 |
| (C═N3)C4CC4)C5═C[N](CC6 | | | | |
| (Cl)CC6)N═C5)C═C2 | | | | |
| CN(C)CC1═NSC(═C1)NC2═NC(═C | <100 | <10 | <100 | <10 |
| (C)C═N2)C3═C[N](CC4(CF)CC4)N═C3 | | | | |
| CC1═C(N═C(NC2═CC═C(CNC | <100 | <10 | <100 | <100 |
| (═O)[C@@H]3CCCN3C(═O)OC(C) | | | | |
| (C)C)C═C2)N═C1)C4═C[N](CC5(CF)CC5)N═C4 | | | | |
| COC1═C(N═C(NC2═CC═C(C═C2)N3CCN | <100 | <10 | <100 | <100 |
| (C)CC3)N═C1)C4═C[N](CC5(CF)CC5)N═C4 | | | | |
| CC1═C(N═C(NC2═CC═C(CN3CCOCC3)C═C2)N═C1) | <100 | <10 | <10 | <100 |
| C4═C[N](CC5(CF)CC5)N═C4 | | | | |
| COC1═C(N═C(NC2═CC═C(C═C2)N3CCN | <100 | <10 | <100 | <100 |
| (C)CC3)N═C1)C4═C[N](CC5(CC5)C#N)N═C4 | | | | |
| C[C@H](N)C(═O)NCC1═CC═C(NC2═NC | <100 | <10 | <100 | <100 |
| (═C(C)C═N2)C3═C[N](CC4 | | | | |
| (CF)CC4)N═C3)C═C1 | | | | |
| CC1═C(N═C(NC2═CC═C(CNC | <100 | <10 | <10 | <100 |
| (═O)[C@@H]3CCCN3)C═C2)N═C1)C4═C[N] | | | | |
| (CC5(CF)CC5)N═C4 | | | | |
| COC1═C(N═C(NC2═CC | <100 | <10 | <100 | <100 |
| (═NS2)CO)N═C1)C3═C[N](CC4(CF)CC4)N═C3 | | | | |
| COC1═NSC(═C1)NC2═NC(═C | <100 | <10 | <100 | <10 |
| (C)C═N2)C3═C[N](CC4(CF)CC4)N═C3 | | | | |
| COCC1═NSC(═C1)NC2═NC(═C | <1k | <100 | <1k | <1k |
| (C)C═N2)C3═C[N](CC4(CF)CC4)N═C3 | | | | |
| CN1CCN(CC1)CC2═CC═C(NC3═NC | <1k | <10 | <100 | <100 |
| (═C(C)C═N3)C4═C[N](CC5 | | | | |
| (CF)CC5)N═C4)C═C2 | | | | |
| CN1CCN(CC1)CC2═NSC(═C2)NC3═NC | <1k | <10 | <100 | <100 |
| (═C(C)C═N3)C4═C[N](CC5 | | | | |
| (CF)CC5)N═C4 | | | | |
| COC1═C(N═C(NC2═CC(═NS2)C)N═C1)C3═C[N] | <100 | <10 | <100 | <100 |
| (CC4(CF)CCC4)N═C3 | | | | |
| COC1═C(N═C(NC2═CC═C(C═C2)N3CCN | <1k | <10 | <100 | <1k |
| (C)CC3)N═C1)C4═C[N](CC5(CF)CCC5)N═C4 | | | | |
| CN(C)CCC1═CC═C(NC2═NC(═C | <1k | <10 | <100 | <100 |
| (C)C═N2)C3═C[N](CC4(CF)CC4)N═C3)C═C1 | | | | |
| CC1═C(N═C(NC2═CC | <100 | <10 | <100 | <10 |
| (═NS2)CN3CCOCC3)N═C1)C4═C[N](CC5 | | | | |
| (CF)CC5)N═C4 | | | | |
| CC1═C(N═C(NC2═CC(═CC═C2)CNC | <100 | <10 | <10 | <100 |
| (═O)OC(C)(C)C)N═C1)C3═C[N](CC4 | | | | |
| (CF)CC4)N═C3 | | | | |
| CN1CC2═C(C1)C═C(NC3═NC(═C | <1k | <10 | <100 | <100 |
| (C)C═N3)C4═C[N](CC5(CF)CCC5)N═C4)C═C2 | | | | |
| CC1═C(N═C(NC2═CC | <100 | <10 | <100 | <100 |
| (═CC═C2)CN)N═C1)C3═C[N](CC4(CF)CC4)N═C3 | | | | |
| CN1CCN(CC1)CC2═CC═CC(═C2)NC3═NC(═C | <1k | <10 | <100 | <1k |
| (C)C═N3)C4═C[N](CC5(CF)CC5)N═C4 | | | | |
| CC1═C(N═C(NC2═CC | <100 | <10 | <10 | <100 |
| (═CC═C2)CN3CCOCC3)N═C1)C4═C[N](CC5 | | | | |
| (CF)CC5)N═C4 | | | | |
| COC1═C(N═C(NC2═CC | <10 | <10 | <10 | <10 |
| (═NS2)C)N═C1)C3═C[N](CC4(CC4)C#N)N═C3 | | | | |
| COCCOC1═CC═C(NC2═NC(═C | <100 | <10 | <100 | <100 |
| (C)C═N2)C3═C[N](CC4(CF)CC4)N═C3)C═C1 | | | | |
| CC1═C(N═C(NC2═CC | <10 | <10 | <10 | <10 |
| (═NS2)CO)N═C1)C3═C[N](CC4(Cl)CC4)N═C3 | | | | |
| COC1═C(N═C(NC2═CC(═NS2)COC | <100 | <10 | <100 | <100 |
| (═O)C3═CC═CC═C3)N═C1)C4═C[N] | | | | |
| (CC5(CF)CC5)N═C4 | | | | |
| CNCC1═NSC(═C1)NC2═NC(═C | <100 | <10 | <100 | <100 |
| (C)C═N2)C3═C[N](CC4(CF)CC4)N═C3 | | | | |
| CC1═C(N═C(NC2═CC | <10 | <10 | <100 | <10 |
| (═NS2)CO)N═C1)C3═C[N](CC4(CC4)C#N)N═C3 | | | | |
| CC1═C(N═C(NC2═CC═C(C═C2)N3CCN | <1k | <10 | <100 | <100 |
| (CC3)C(═O)OC(C)(C)C)N═C1)C4═C[N] | | | | |
| (CC5(CF)CC5)N═C4 | | | | |
| COCCOCC1═NSC(═C1)NC2═NC(═C | <1k | <1k | <1k | <1k |
| (C)C═N2)C3═C[N](CC4(CF)CC4)N═C3 | | | | |
| CN1CCN(CC1)CCC(═O)OCC2═NSC | <100 | <10 | <10 | <10 |
| (═C2)NC3═NC(═C(C)C═N3)C4═C[N] | | | | |
| (CC5(CF)CC5)N═C4 | | | | |
| CN(C)CC(═O)NCC1═CC═CC(═C1)NC2═NC | <100 | <10 | <10 | <100 |
| (═C(C)C═N2)C3═C[N](CC4 | | | | |
| (CF)CC4)N═C3 | | | | |
| CN(C)C1CN(C1)C2═CC═C(NC3═NC | <100 | <10 | <10 | <100 |
| (═C(C)C═N3)C4═C[N](CC5 | | | | |
| (CF)CC5)N═C4)C═C2 | | | | |
| CC1═C(N═C(NC2═CC═C(C═C2)N3CC4 | <100 | <10 | <10 | <100 |
| (COC4)C3)N═C1)C5═C[N](CC6(CF)CC6)N═C5 | | | | |
| CC1═C(N═C(NC2═CC═C | <100 | <10 | <100 | <100 |
| (CO)N═C2)N═C1)C3═C[N](CC4(CF)CC4)N═C3 | | | | |
| COC1═CC═C(NC2═NC(═C | <1k | <10 | <100 | <100 |
| (C)C═N2)C3═C[N](CC4(CF)CC4)N═C3)C═N1 | | | | |
| CN(C)CCOC1═CC(═CC═N1)NC2═NC | <100 | <10 | <100 | <100 |
| (═C(C)C═N2)C3═C[N](CC4(CF)CC4)N═C3 | | | | |
| CC1═C(N═C(NC2═CC | <100 | <10 | <100 | <100 |
| (═NS2)CN)N═C1)C3═C[N](CC4(CF)CC4)N═C3 | | | | |
| COC1═C(N═C(NC2═CC | <100 | <10 | <100 | <100 |
| (═NS2)CO)N═C1)C3═C[N](CC4(Cl)CC4)N═C3 | | | | |
| CN(C)CCC(═O)OCC1═NSC(═C1)NC2═NC | <100 | <10 | <10 | <10 |
| (═C(C)C═N2)C3═C[N](CC4(CF)CC4)N═C3 | | | | |
| CC1═C(N═C(NC2═CC(═NS2)COC | <100 | <10 | <10 | <10 |
| (═O)CCN3CCOCC3)N═C1)C4═C[N](CC5 | | | | |
| (CF)CC5)N═C4 | | | | |
| CC1═C(N═C(NC2═CC(═CC═C2)CNC | <100k | <100 | <1k | <10k |
| (═O)[C@H](CCCN\C(NC(═O)OC(C) | | | | |
| (C)C)═N\C(═O)OC(C)(C)C)NC(═O)OC | | | | |
| (C)(C)C)N═C1)C3═C[N](CC4(CF)CC4)N═C3 | | | | |
| CC1═C(N═C(NC2═CC═C | <100 | <10 | <10 | <10 |
| (C═C2)N3CCNCC3)N═C1)C4═C[N](CC5 | | | | |
| (CF)CC5)N═C4 | | | | |
| COC1═C(N═C(NC2═CC═NS2)N═C1)C3═C[N] | <100 | <10 | <100 | <100 |
| (CC4(CC4)C#N)N═C3 | | | | |
| CC1═C(N═C(NC2═CC(═CC═C2)CNC | <1k | <10 | <100 | <100 |
| (═O)[C@@H](N)CCCNC | | | | |
| (N)═N)N═C1)C3═C[N](CC4(CF)CC4)N═C3 | | | | |
| COCCOC1═CC═C(NC2═NC(═C | <100 | <10 | <100 | <100 |
| (C)C═N2)C3═C[N](CC4(CF)CC4)N═C3)C═N1 | | | | |
| COC1═C(N═C(NC2═CC(═NS2)CO)N═C1)C3═C[N] | <10 | <10 | <100 | <10 |
| (CC4(CC4)C#N)N═C3 | | | | |
| CC1═NSC(═C1)NC2═NC(═C | <10 | <10 | <10 | <10 |
| (C)C═N2)C3═C[N](CC4(CC4)CC#N)N═C3 | | | | |
| CC1═C(N═C(NC2═CC═NS2)N═C1)C3═C[N] | <10 | <10 | <100 | <10 |
| (CC4(CC4)C#N)N═C3 | | | | |
| ClC1═C(N═C(NC2═CC═NS2)N═C1)C3═C[N] | <100 | <10 | <100 | <100 |
| (CC4(CCC4)C#N)N═C3 | | | | |
| COC1═C(N═C(NC2═CC═NS2)N═C1)C3═C[N] | <1k | <10 | <100 | <1k |
| (CC4(CF)CCC4)N═C3 | | | | |
| CC1═NSC(═C1)NC2═NC(═C | <100 | <10 | <10 | <10 |
| (C)C═N2)C3═C[N](CC(C)(C)CF)N═C3CN | | | | |
| (C)CCOC1═CC═C(NC2═NC | <100 | <10 | <100 | <100 |
| (═C(C)C═N2)C3═C[N](CC4 | | | | |
| (CF)CC4)N═C3)C═C1 | | | | |
| CC1═C(N═C(NC2═CC(═C(C═C2)N3CC4 | <100 | <10 | <10 | <10 |
| (COC4)C3)F)N═C1)C5═C[N](CC6 | | | | |
| (CF)CC6)N═C5 | | | | |
| C[N]1C═C(NC2═NC(═C(C)C═N2)C3═C[N] | <1k | <100 | <1k | <1k |
| (CC4(CF)CC4)N═C3)N═N1 | | | | |
| CN(C)CC1═CC(═CC═N1)NC2═NC(═C | <1k | <10 | <100 | <100 |
| (C)C═N2)C3═C[N](CC4(CF)CCC4)N═C3 | | | | |
| COC(═O)C1═CC═C(NC2═NC(═C | <100 | <10 | <100 | <100 |
| (C)C═N2)C3═C[N](CC4 | | | | |
| (CF)CC4)N═C3)C═C1 | | | | |
| COC1═C(N═C(NC2═CC═NS2)N═C1)C3═C[N] | <100 | <10 | <100 | <100 |
| (CC4(CF)CC4)N═C3 | | | | |
| CN(C)C(═O)C1═CC═C(NC2═NC(═C | <100 | <10 | <100 | <100 |
| (C)C═N2)C3═C[N](CC4(CF)CC4)N═C3)C═C1 | | | | |
| COC1═C(N═C(NC2═CC═NS2)N═C1)C3═C[N] | <100 | <10 | <100 | <100 |
| (CC4(Cl)CC4)N═C3 | | | | |
| COC1═C(N═C(NC2═CC(═NS2)C)N═C1)C3═C[N] | <100 | <10 | <100 | <100 |
| (CC4(CC4)CC#N)N═C3 | | | | |
| COC(═O)C1═CC(═CC═C1)NC2═NC(═C | <100 | <10 | <10 | <100 |
| (C)C═N2)C3═C[N](CC4(CF)CC4)N═C3 | | | | |
| CN(C)C(═O)C1═CC(═CC═C1)NC2═NC | <100 | <10 | <100 | <100 |
| (═C(C)C═N2)C3═C[N](CC4 | | | | |
| (CF)CC4)N═C3 | | | | |
| CC1═C(N═C(NC2═CC═CC(═C2)C | <100 | <10 | <100 | <100 |
| (N)═O)N═C1)C3═C[N](CC4 | | | | |
| (CF)CC4)N═C3 | | | | |
| CC1═C(N═C(NC2═CC═C(C═C2)C | <10 | <10 | <10 | <10 |
| (N)═O)N═C1)C3═C[N](CC4 | | | | |
| (CF)CC4)N═C3 | | | | |
| CN(C)CCOC1═CC═C(NC2═NC(═C | <1k | <10 | <100 | <100 |
| (C)C═N2)C3═C[N](CC4(CF)CC4)N═C3)C═C1F | | | | |
| CN(C)CCOC1═CC(═CC═N1)NC2═NC(═C | <100 | <10 | <100 | <100 |
| (C)C═N2)C3═C[N](CC4(CC4)CC#N)N═C3 | | | | |
| COC1(COC1)C2═CC═C(NC3═NC(═C | 1879.697 | <100 | <1k | <1k |
| (C)C═N3)C4═C[N](CC5(CF)CC5)N═C4)C═C2 | | | | |
| CC1═C(N═C(NC2═CC═NS2)N═C1)C3═C[N] | <10 | <10 | <10 | <10 |
| (CC4(CC4)CC#N)N═C3 | | | | |
| CN(C)CCC(═O)NCC1═CC═C(NC2═NC(═C | <100 | <10 | <100 | <100 |
| (C)C═N2)C3═C[N](CC4(CF)CC4)N═C3)C═C1 | | | | |
| CC1═C(N═C(NC2═CC═C(CNC | <100 | <10 | <100 | <100 |
| (═O)CCN3CCOCC3)C═C2)N═C1)C4═C[N] | | | | |
| (CC5(CF)CC5)N═C4 | | | | |
| CNC(═O)C1═CC═C(NC2═NC(═C | <10 | <10 | <10 | <10 |
| (C)C═N2)C3═C[N](CC4(CF)CC4)N═C3)C═C1 | | | | |
| CC1═NSC(═C1)NC2═NC(═CC═N2)C3═C[N] | <100 | <10 | <100 | <10 |
| (CC4(CC4)CC#N)N═C3 | | | | |
| CN1CCOC2═CC(═CC═C12)NC3═NC(═C | <100 | <10 | <100 | <100 |
| (C)C═N3)C4═C[N](CC5(CF)CC5)N═C4 | | | | |
| CC1═C(N═C(NC2═CC═C(C═C2)C3 | <100 | <10 | <10 | <10 |
| (O)COC3)N═C1)C4═C[N](CC5 | | | | |
| (CF)CC5)N═C4 | | | | |
| OCC1═NSC(═C1)NC2═NC(═C | <100 | <10 | <100 | <100 |
| (C═N2)C3CC3)C4═C[N](CC5 | | | | |
| (CC5)C#N)N═C4 | | | | |
| OCC1═NSC(═C1)NC2═NC(═C | <100 | <10 | <100 | <100 |
| (C═N2)C3CC3)C4═C[N](CC5(CF)CC5)N═C4 | | | | |
| CC1═C(N═C(NC2═CC(═CC═C2)C | <100 | <10 | <100 | <10 |
| (O)CO)N═C1)C3═C[N](CC4(CF)CC4)N═C3 | | | | |
| CN(C)CCNC(═O)C1═CC═C(NC2═NC | <100 | <10 | <100 | <10 |
| (═C(C)C═N2)C3═C[N](CC4 | | | | |
| (CF)CC4)N═C3)C═C1 | | | | |
| OCC1═NSC(═C1)NC2═NC(═CC═N2)C3═C[N] | <100 | <10 | <100 | <10 |
| (CC4(CC4)CC#N)N═C3 | | | | |
| COC1═C(N═C(NC2═CC═NS2)N═C1)C3═C[N] | <100 | <10 | <100 | <100 |
| (CC4(CC4)CC#N)N═C3 | | | | |
| N#CCC1(CC1)C[N]2C═C(C═N2)C3═C | <100 | <10 | <100 | <100 |
| (C═NC(═N3)NC4═CC═NS4)C5CC5 | | | | |
| CC1═NSC(═C1)NC2═NC(═C | <100 | <10 | <100 | <100 |
| (C═N2)C3CC3)C4═C[N](CC5(CC5)CC#N)N═C4 | | | | |
| COC1═C(N═C(NC2═CC(═NS2)CO)N═C1)C3═C[N] | <1k | <100 | <1k | <1k |
| (CC4(CC4)CC#N)N═C3 | | | | |
| OCC1═NSC(═C1)NC2═NC(═C | <100 | <10 | <100 | <100 |
| (C═N2)C3CC3)C4═C[N](CC5(CC5)CC#N)N═C4 | | | | |
| CC1═C(N═C(NC2═CC═C(C═C2)C3 | <100k | <1k | <100k | <100k |
| (N)COC3)N═C1)C4═C[N](CC5(CF)CC5)N═C4 | | | | |
| CC1═NSC(═C1)NC2═NC(═CC═N2)C3═C[N] | <100 | <10 | <100 | <10 |
| (CC4(COC4)CC#N)N═C3 | | | | |
| CC1═C(N═C(NC2═CC═C(C═C2)C | <100 | <10 | <100 | <10 |
| (O)═O)N═C1)C3═C[N](CC4(CF)CC4)N═C3 | | | | |
| CC1═C(N═C(NC2═CC═CC(═C2)C | <1k | <10 | <100 | <1k |
| (O)═O)N═C1)C3═C[N](CC4(CF)CC4)N═C3 | | | | |
| CC1═C(N═C(NC2═CC═CC(═C2)C3 | <1k | <10 | <100 | <1k |
| (O)COC3)N═C1)C4═C[N](CC5(CF)CC5)N═C4 | | | | |
| CC1═C(N═C(NC2═CC(═CC═C2)CCO)N═C1)C3═C[N] | <10 | <10 | <10 | <10 |
| (CC4(CF)CC4)N═C3 | | | | |
| COC1═C(N═C(NC2═CC═C(C═C2)N3CCN | <1k | <10 | <100 | <1k |
| (C)CC3)N═C1)C4═C[N](CC5(CC5)CC#N)N═C4 | | | | |
| CN1CCN(CC1)C2═C(C)C═C(NC3═NC | <100 | <10 | <10 | <10 |
| (═C(C)C═N3)C4═C[N](CC5 | | | | |
| (CF)CC5)N═C4)C═C2 | | | | |
| CN(C)CCC(═O)NCC1═NSC(═C1)NC2═NC | <10 | <10 | <10 | <10 |
| (═C(C)C═N2)C3═C[N](CC4(CF)CC4)N═C3 | | | | |
| N#CCC1(CC1)C[N]2C═C(C═N2)C3═CC═NC | <100 | <10 | <100 | <100 |
| (═N3)NC4═CC═NS4 | | | | |
| COC1═C(N═C(NC2═CC═C(C═C2)C3 | <1k | <10 | <100 | <100 |
| (O)COC3)N═C1)C4═C[N](CC5 | | | | |
| (CF)CC5)N═C4 | | | | |
| CC1═C(N═C(NC2═CC(═C | <100 | <10 | <10 | <10 |
| (C═C2)N3CCOCC3)C)N═C1)C4═C[N] | | | | |
| (CC5(CF)CC5)N═C4 | | | | |
| OCC(O)C1═CC═CC(═C1)NC2═NC(═C | <10 | <10 | <10 | <10 |
| (Cl)C═N2)C3═C[N](CC4(CF)CC4)N═C3 | | | | |

*Anderson, E., G. D. Veith, and D. Weininger. 1987. SMILES: A line notation and computerized interpreter for chemical structures. Report No. EPA/600/M-87/021. U.S. Environmental Protection Agency, Environmental Research Laboratory-Duluth, Duluth, MN 55804.

In some embodiments, the compounds described herein are selected from a compound of Table 1, or a pharmaceutically acceptable salt thereof.

The compounds provided herein may possess one or more stereocenters, and each stereocenter may exist independently in either the R or S configuration. In some embodiments, the compounds provided herein are present in optically active or racemic forms. It is to be understood that the compounds provided herein encompass racemic, optically-active, regioisomeric, and stereoisomeric forms, or combinations thereof that possess the therapeutically useful properties provided herein.

Preparation of optically active forms can be achieved in any suitable manner, including by way of non-limiting example, by resolution of the racemic form with recrystallization techniques, synthesis from optically-active starting materials, chiral synthesis, or chromatographic separation using a chiral stationary phase. In some embodiments, a mixture of one or more isomer can be utilized as the therapeutic compound provided herein. In some embodiments, compounds provided herein contain one or more chiral centers. The compounds provided herein can be prepared by any means, including stereoselective synthesis, enantioselective synthesis or separation of a mixture of enantiomers or diastereomers. Resolution of compounds and isomers thereof can be achieved by any means including, by way of non-limiting example, chemical processes, enzymatic processes, fractional crystallization, distillation, or chromatography.

In some embodiments, the compounds provided herein exist as tautomers. All tautomers may be included within the scope of the compounds presented herein.

In some embodiments, the compounds provided herein also include isotopically-labeled compounds wherein one or more atoms is replaced by an atom having the same atomic number, but an atomic mass or mass number different from the atomic mass or mass number most frequently occurring in nature. Examples of isotopes suitable for inclusion in the compounds provided herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In some embodiments, isotopically-labeled compounds are useful in drug or substrate tissue distribution studies. In some embodiments, substitution with heavier isotopes such as deuterium affords greater metabolic stability (for example, increased in vivo half-life or reduced dosage requirements). In some embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In some embodiments, the compounds provided herein are labeled by other means, including, but not limited to, the use of chromophores or fluorescent moieties, bioluminescent labels, or chemiluminescent labels.

The compounds provided herein, and other related compounds having different substituents are synthesized using techniques and materials provided herein and as described, for example, in: Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); Larock's Comprehensive Organic Transformations (VCH Publishers Inc., 1989); March, Advanced Organic Chemistry 4th Ed., (Wiley 1992); Carey and Sundberg, Advanced Organic Chemistry 4th Ed., Vols. A and B (Plenum 2000, 2001); and Green and Wuts, Protective Groups in Organic Synthesis 3rd Ed., (Wiley 1999) (all of which are incorporated herein in their entirety by reference for such disclosure). General methods for the preparation of compound as provided herein are modified by the use of appropriate reagents and conditions, for the introduction of the various moieties found in the formulae as provided herein.

Compounds provided herein are synthesized using any suitable procedures starting from compounds that are available from commercial sources, or are prepared using procedures provided herein.

In some embodiments, reactive functional groups, such as hydroxyl, amino, imino, thio, or carboxy groups, are protected in order to avoid their unwanted participation in reactions. Protecting groups are used to block some or all of the reactive moieties and prevent such groups from participating in chemical reactions until the protective group is removed. In some embodiments, each protective group is removable by a different means. Protective groups that are cleaved under totally disparate reaction conditions fulfill the requirement of differential removal.

In some embodiments, protective groups are removed by acid, base, reducing conditions (such as, for example, hydrogenolysis), or oxidative conditions. For example, groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

### Methods

The compounds disclosed herein, and compositions including them, have kinase inhibitory activity, and are thus useful in modulating the action of kinases, and in treatment or prevention of diseases or conditions influenced by kinases. The compounds and compositions provided herein may be used to modulate the action of kinases either in a cell in vitro or in a cell in a living body in vivo. In some embodiments, provided herein are methods of inhibiting the action of a kinase comprising applying to a medium such as an assay medium or contacting with a cell either in a cell in vitro or in a cell in a living body in vivo a therapeutically effective inhibitory amount of a compound or composition described herein. In some embodiments, the kinase inhibited is a JAK kinase, such as JAK1, JAK2, JAK3, TYK2, or a combination thereof. In some embodiments, the compounds and compositions provided herein are useful as JAK-inhibitors. Accordingly, in some embodiments the compounds and compositions provided herein are useful in treating JAK-associated diseases or disorders.

Examples of JAK-associated diseases include diseases involving the immune system including, for example, organ transplant rejection (e.g., allograft rejection and graft versus host disease). Further examples of JAK-associated diseases include autoimmune diseases such as multiple sclerosis, rheumatoid arthritis, juvenile arthritis, psoriatic arthritis, type I diabetes, lupus, psoriasis, inflammatory bowel disease, ulcerative colitis, Crohn's disease, myasthenia gravis, immunoglobulin nephropathies, myocarditis, autoimmune thyroid disorders, chronic obstructive pulmonary disease (COPD), and the like. In some embodiments, the autoimmune disease is arthritis.

Further examples of JAK-associated diseases include allergic conditions such as asthma, food allergies, eczematous dermatitis, contact dermatitis, atopic dermatitis (atropic eczema), and rhinitis. Further examples of JAK-associated diseases include viral diseases such as Epstein Barr Virus (EBV), Hepatitis B, Hepatitis C, HIV, HTLV 1, Varicella-Zoster Virus (VZV) and Human Papilloma Virus (HPV).

Further examples of JAK-associated diseases or conditions include those characterized by solid tumors (e.g., prostate cancer, renal cancer, hepatic cancer, pancreatic cancer, gastric cancer, breast cancer, lung cancer, cancers of the head and neck, thyroid cancer, glioblastoma, Kaposi's sarcoma, Castleman's disease, uterine leiomyosarcoma, melanoma etc.), hematological cancers (e.g., lymphoma, leukemia such as acute lymphoblastic leukemia (ALL), acute myelogenous leukemia (AML) or multiple myeloma), and skin cancer such as cutaneous T-cell lymphoma (CTCL) and cutaneous B-cell lymphoma. Example CTCLs include Sezary syndrome and mycosis fungoides. Other examples of JAK-associated diseases or conditions include pulmonary arterial hypertension.

Other examples of JAK-associated diseases or conditions include inflammation-associated cancers. In some embodiments, the cancer is associated with inflammatory bowel disease. In some embodiments, the inflammatory bowel disease is ulcerative colitis. In some embodiments, the inflammatory bowel disease is Crohn's disease. In some embodiments, the inflammation-associated cancer is colitis-associated cancer. In some embodiments, the inflammation-associated cancer is colon cancer or colorectal cancer. In some embodiments, the cancer is gastric cancer, gastrointestinal carcinoid tumor, gastrointestinal stromal tumor (GIST), adenocarcinoma, small intestine cancer, or rectal cancer.

Thus, in one embodiment, provided herein are methods of treating a JAK-associated disease in a subject in need thereof, comprising administering a therapeutically effective amount or dose of a compound or composition provided herein to the subject.

In some embodiments, provided herein are methods of treating a JAK-associated disease in a subject in need thereof, comprising administering to the subject a compound provided in Table 1, or a pharmaceutically acceptable salt thereof. In some embodiments, provided herein are methods of treating a JAK-associated disease in a subject in need thereof, comprising administering to the subject a composition, such as a pharmaceutical composition, comprising a compound provided in Table 1, or a pharmaceutically acceptable salt thereof.

In some embodiments, the JAK-associated diseases include diseases involving the immune system. In some embodiments, the JAK-associated diseases include organ transplant rejection, such as allograft rejection or graft-versus-host disease. In some embodiments, the JAK-associated diseases include autoimmune diseases, such as multiple sclerosis, rheumatoid arthritis, juvenile arthritis, type I diabetes, lupus, psoriasis, inflammatory bowel disease, ulcerative colitis, Crohn's disease, myasthenia gravis, immunoglobulin nephropathies, and autoimmune thyroid disorders. In some embodiments, the autoimmune disease is an autoimmune bullous skin disorder such as pemphigus vulgaris (PV) or bullous pemphigoid (BP).

In some embodiments, the JAK-associated diseases include allergic conditions. In some embodiments, the JAK-associated diseases include asthma, food allergies, atopic dermatitis, and rhinitis.

In some embodiments, the JAK-associated diseases include viral diseases. In some embodiments, the JAK-associated diseases include Epstein Barr Virus (EBV), Hepatitis B, Hepatitis C, HIV, HTLV 1, Varicella-Zoster Virus (VZV), Human Papilloma Virus (HPV), and SARS-COV-2 virus.

In some embodiments, the JAK-associated diseases include skin disorders. In some embodiments, the JAK-associated diseases include psoriasis (for example, psoriasis vulgaris), atopic dermatitis, skin rash, skin irritation, skin sensitization such as contact dermatitis or allergic contact dermatitis. Certain substances, including some pharmaceuticals when topically applied, can cause skin sensitization. In some embodiments, co-administration or sequential administration of at least one compound or composition provided herein together with the agent causing unwanted sensitization can be helpful in treating such unwanted sensitization or dermatitis. In some embodiments, the skin disorder is treated by topical administration of at least one compound or composition provided herein.

In some embodiments, the JAK-associated diseases include cancers. In some embodiments, the JAK-associated diseases include solid tumors such as prostate cancer, renal cancer, hepatic cancer, pancreatic cancer, gastric cancer, breast cancer, lung cancer, cancers of the head and neck, thyroid cancer, glioblastoma, Kaposi's sarcoma, Castleman's disease, and melanoma. In some embodiments, the JAK-associated diseases include hematological cancers such as lymphoma, leukemia such as acute lymphoblastic leukemia (ALL), acute myelogenous leukemia (AML), chronic myelogenous leukemia (CML), or multiple myeloma. In some embodiments, the JAK-associated diseases include myeloproliferative diseases or conditions such as essential thrombocythemia, polycythemia vera, myelofibrosis, post-essential thrombocythemia, post-polycythemia vera, systemic mastocytosis, hypereosinophilic syndrome (HES), erythrocytosis, leukocytosis, thrombocytosis, polycythemia, chronic myelomonocytic leukemia (CMML), myelodysplastic syndrome, myeloid metaplasia with myelofibrosis (MMM), and acute myeloid leukemia. In some embodiments, the JAK-associated diseases include skin cancers such as cutaneous T-cell lymphoma (CTCL) and cutaneous B-cell lymphoma. Examples of cutaneous T-cell lymphomas include Sézary syndrome and mycosis fungoides.

In some embodiments, the JAK-associated diseases include those characterized by expression of a mutant JAK2. In some embodiments, the JAK-associated diseases include those associated with a JAK having at least one mutation in the pseudo-kinase domain, such as JAK2V617F.

In some embodiments, the JAK-associated diseases include inflammation and inflammatory diseases. In some embodiments, the JAK-associated diseases include inflammatory diseases of the eye such as iritis, uveitis, scleritis, conjunctivitis, and related disease. In some embodiments, the JAK-associated diseases include inflammatory diseases of the respiratory tract such as those of the upper respiratory tract including the nose and sinuses such as rhinitis or sinusitis or the lower respiratory tract including bronchitis, and chronic obstructive pulmonary disease. In some embodiments, the JAK-associated diseases include inflammatory myopathy such as myocarditis, and other inflammatory diseases. Other inflammatory diseases treatable by the compounds or compositions provided herein include systemic inflammatory response syndrome (SIRS) or septic shock.

The compounds of the present disclosure are used in methods of inhibiting kinases in a cell, a tissue or a subject such as a human comprising contacting the cell with an amount of one or more of the compounds of the present disclosure therapeutically effective to inhibit the kinase. In one embodiment, the compounds are administered in a pharmaceutically acceptable composition, such as in or with a pharmaceutically acceptable carrier.

In another embodiment, the compounds of the present disclosure are used in methods for modulating the action of a kinase in a cell comprising contacting the cell with amount of one or more compounds of the present disclosure therapeutically effective to modulate the action of a kinase in a cell. In one embodiment, the compounds of the present disclosure are administered in a pharmaceutically acceptable composition, such as in or with a pharmaceutically acceptable carrier.

Treatment or prevention of diseases or conditions for which the compounds of the present disclosure may be useful includes any of the diseases or conditions associated with kinase activity or diseases or conditions affected by kinases. Examples of these types of diseases include neurodegenerative diseases, such as Alzheimer's; ocular diseases, such as diabetic eye diseases, wet age-related macular degeneration, or dry age-related macular degeneration, inflammatory eye diseases, retinal degradation and glaucoma; cardiovascular diseases; and cancer. Additional examples include bone disorder, obesity, hepatic disease, renal disease, pancreatitis, gastric disturbance, hypertension, fertility control, disorders of hair growth, nasal congestion, neurogenic bladder disorder, gastrointestinal disorder, dermatological disorder, and respiratory indications.

In some embodiments, the compounds of the present disclosure will be administered in conjunction with one or more additional therapeutic agents. Suitable classes of additional therapeutic agents include, but are not limited to, beta blockers, alpha-agonists, carbonic anhydrase inhibitors, prostaglandin-like compounds, miotic or cholinergic agents, epinephrine compounds, or neuroprotective or anti-inflammatory compounds.

Beta blockers. These compounds are thought to lower intraocular pressure (IOP) by reducing the production of aqueous humor. Examples include levobunolol (BETAGAN™), timolol (BETIMOL™, TIMOPTIC™), betaxolol (BETOPTIC™) and metipranolol (OPTIPRANOLOL™).

Alpha-agonists. These compounds are thought to lower IOP by reducing the production of aqueous humor and increasing drainage. Examples include apraclonidine (IOPIDINE™) and brimonidine (ALPHAGAN™).

Carbonic anhydrase inhibitors. These compounds are thought to lower IOP by also reducing the production of aqueous humor. Examples include dorzolamide (TRUSOPT™) and brinzolamide (AZOPT™).

Prostaglandin-like compounds. These compounds are thought to lower IOP by increasing the outflow of aqueous humor by the uveoscleral pathway. Examples include AR-102, latanoprost (XALATAN™), bimatoprost (LUMIGAN™), tafluprost (ZIOPTAN™), and travoprost (TRAVATAN™).

Miotic or cholinergic agents. These agents are thought to function by causing the pupil to constrict, which opens drainage channels in the eye. Examples include pilocarpine (ISOPTO CARPINE™, PILOPINE™) and carbachol (ISOPTO CARBACHOL™).

Epinephrine compounds. These compounds, such as dipivefrin (PROPINE™), are thought to function by both decreasing the outflow of aqueous humor, as well as increasing fluid drainage.

Neuroprotective or anti-inflammatory compounds. These compounds, such as Aflibercept (EYLEA™) are treatments for conditions of the retina such as Macular Degeneration, and are designed as anti-VEGF treatments or have similar types of anti-growth or anti-inflammatory activity.

Also provided herein are methods of treating an ocular disorder in a subject in need thereof, comprising administering to the subject a compound, a composition, or a pharmaceutical composition provided herein.

Also provided herein are methods of reducing intraocular pressure in a subject in need thereof, comprising administering to the subject a compound, a composition, or a pharmaceutical composition provided herein.

In some embodiments, provided herein are methods of treating an ocular disorder in a subject in need thereof, comprising administering to the subject a compound, or a pharmaceutically acceptable salt thereof, provided herein.

In some embodiments, the ocular disorder is glaucoma.

In some embodiments, provided herein are methods of reducing intraocular pressure in a subject in need thereof, comprising administering to the subject a compound, or a pharmaceutically acceptable salt thereof, provided herein.

In some embodiments, the compound is administered topically to the subject, such as to a mucus membrane of the subject, to the skin of the subject, or to an eye of the subject.

In some embodiments, provided herein are methods of treating an ocular disorder in a subject in need thereof, comprising administering to the subject a compound of one of the Formulae provided herein, or a pharmaceutically acceptable salt thereof.

In some embodiments, provided herein are methods of treating an ocular disorder in a subject in need thereof, comprising administering to the subject a compound provided in Table 1, or a pharmaceutically acceptable salt thereof.

In some embodiments, provided herein are methods of reducing intraocular pressure in a subject in need thereof, comprising administering to the subject a compound of one of the Formulae provided herein, or a pharmaceutically acceptable salt thereof.

In some embodiments, provided herein are methods of reducing intraocular pressure in a subject in need thereof, comprising administering to the subject a compound provided in Table 1, or a pharmaceutically acceptable salt thereof.

In some embodiments of these methods, the method further comprises administering one or more additional therapeutic agents. In some embodiments, the one or more additional therapeutic agents is a beta blocker, an alpha-agonist, a carbonic anhydrase inhibitor, a prostaglandin or a prostaglandin-like compound, a miotic or cholinergic agent, an epinephrine compound, or a neuroprotective or anti-inflammatory compound. In some embodiments, the one or more additional therapeutic agents is a prostaglandin or a prostaglandin-like compound. In some embodiment, the prostaglandin-like compound is AR-102, latanoprost, bimatoprost, tafluprost, or travoprost.

Also provided herein are methods of treating an autoimmune disease in a subject in need thereof, comprising administering to the subject a compound, a composition, or a pharmaceutical composition provided herein.

In some embodiments, provided herein are methods of treating an autoimmune disease in a subject in need thereof, comprising administering to the subject a compound of one of the Formulae provided herein, or a pharmaceutically acceptable salt thereof.

In some embodiments, provided herein are methods of treating an autoimmune disease in a subject in need thereof, comprising administering to the subject a compound provided in Table 1, or a pharmaceutically acceptable salt thereof.

In some embodiments, the autoimmune disease is multiple sclerosis, rheumatoid arthritis, juvenile arthritis, psoriatic arthritis, type I diabetes, lupus, psoriasis, inflammatory bowel disease, ulcerative colitis, Crohn's disease, myasthenia gravis, immunoglobulin nephropathies, myocarditis, autoimmune thyroid disorders, or chronic obstructive pulmonary disease.

### Compositions and Administration

The additional therapeutic agent or agents can be administered simultaneously or sequentially with the compounds of the present disclosure. Sequential administration includes administration before or after the compounds of the present disclosure. In some embodiments, the additional therapeutic agent or agents can be administered in the same composition as the compounds of the present disclosure. In other embodiments, there can be an interval of time between administration of the additional therapeutic agent and the compounds of the present disclosure.

In some embodiments, the administration of an additional therapeutic agent with a compound of the present disclosure will enable lower doses of the other therapeutic agents to be administered for a longer period of time.

Also provided herein are compositions comprising a compound provided herein, or a pharmaceutically acceptable salt thereof. In one embodiment, the compositions provided herein are pharmaceutical compositions comprising a pharmaceutically acceptable carrier.

Pharmaceutical compositions for use in accordance with the present disclosure may be formulated in a conventional manner using one or more physiologically acceptable carriers or excipients. Thus, the compounds and their physiologically acceptable salts and solvates may be formulated for administration by, for example, solid dosing, eyedrop, in a topical oil-based formulation, injection (including injection of a drug-eluting device either into the body as a whole, or into specific tissues of the eye), inhalation (either through the mouth or the nose), implants, or oral, buccal, parenteral or rectal administration. Techniques and formulations may generally be found in “Remington's Pharmaceutical Sciences,” (Meade Publishing Co., Easton, PA).

The route by which the compounds of the present disclosure (component A) will be administered and the form of the composition will dictate the type of carrier (component B) to be used. The composition may be in a variety of forms, suitable, for example, for systemic administration (e.g., oral, rectal, nasal, sublingual, buccal, implants, or parenteral, or by ocular injection into one of the chambers of the eye, such as intravitreal injection, intracameral injection, or injection into the aqueous humour.) or topical administration (e.g., local application on the skin, ocular, liposome delivery systems, or iontophoresis).

Carriers for systemic administration typically comprise at least one of a) diluents, b) lubricants, c) binders, d) disintegrants, e) colorants, f) flavors, g) sweeteners, h) antioxidants, j) preservatives, k) glidants, m) solvents, n) suspending agents, o) wetting agents, p) surfactants, combinations thereof, and others. All carriers are optional in the systemic compositions.
Ingredient a) is a diluent. Suitable diluents for solid dosage forms include sugars such as glucose, lactose, dextrose, and sucrose; diols such as propylene glycol; calcium carbonate; sodium carbonate; sugar alcohols, such as glycerin; mannitol; and sorbitol. The amount of ingredient a) in the systemic or topical composition is typically about 50 to about 90%.
Ingredient b) is a lubricant. Suitable lubricants for solid dosage forms are exemplified by solid lubricants including silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of Theobroma. The amount of ingredient b) in the systemic or topical composition is typically about 5 to about 10%.
Ingredient c) is a binder. Suitable binders for solid dosage forms include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of ingredient c) in the systemic composition is typically about 5 to about 50%, and in ocular solid dosing forms up to 99%.
Ingredient d) is a disintegrant. Suitable disintegrants for solid dosage forms include agar, alginic acid and the sodium salt thereof, effervescent mixtures, croscarmelose, crospovidone, sodium carboxymethyl starch, sodium starch glycolate, clays, and ion exchange resins. The amount of ingredient d) in the systemic or topical composition is typically about 0.1 to about 10%.
Ingredient e) for solid dosage forms is a colorant such as an FD&C dye. When used, the amount of ingredient e) in the systemic or topical composition is typically about 0.005 to about 0.1%.
Ingredient f) for solid dosage forms is a flavor such as menthol, peppermint, and fruit flavors. The amount of ingredient f), when used, in the systemic or topical composition is typically about 0.1 to about 1.0%.
Ingredient g) for solid dosage forms is a sweetener such as aspartame and saccharin. The amount of ingredient g) in the systemic or topical composition is typically about 0.001 to about 1%.
Ingredient h) is an antioxidant such as butylated hydroxyanisole (“BHA”), butylated hydroxytoluene (“BHT”), and vitamin E. The amount of ingredient h) in the systemic or topical composition is typically about 0.1 to about 5%.
Ingredient j) is a preservative such as benzalkonium chloride, methyl paraben and sodium benzoate. The amount of ingredient j) in the systemic or topical composition is typically about 0.01 to about 5%.
Ingredient k) for solid dosage forms is a glidant such as silicon dioxide. The amount of ingredient k) in the systemic or topical composition is typically about 1 to about 5%.
Ingredient m) is a solvent, such as water, isotonic saline, ethyl oleate, glycerine, hydroxylated castor oils, alcohols such as ethanol, and phosphate buffer solutions. The amount of ingredient m) in the systemic or topical composition is typically from about 0 to about 100%.
Ingredient n) is a suspending agent. Suitable suspending agents include AVICEL® RC-591 (from FMC Corporation of Philadelphia, PA) and sodium alginate. The amount of ingredient n) in the systemic or topical composition is typically about 1 to about 8%.
Ingredient o) is a surfactant such as lecithin, Polysorbate 80, and sodium lauryl sulfate, and the TWEENS® from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and Mccutcheon's Volume 1, Emulsifiers & Detergents, 1994, North American Edition, pp. 236-239. The amount of ingredient o) in the systemic or topical composition is typically about 0.1% to about 5%.

Although the amounts of components A and B in the systemic compositions will vary depending on the type of systemic composition prepared, the specific derivative selected for component A and the ingredients of component B, in general, system compositions comprise 0.01% to 50% of component A and 50 to 99.99% of component B.

Compositions for parenteral administration typically comprise A) 0.1 to 10% of the compounds of the present disclosure and B) 90 to 99.9% of a carrier comprising a) a diluent and m) a solvent. In one embodiment, component a) comprises propylene glycol and m) comprises ethanol or ethyl oleate.

Compositions for oral administration can have various dosage forms. For example, solid forms include tablets, capsules, granules, and bulk powders. These oral dosage forms comprise a safe and therapeutically effective amount, usually at least about 5%, and more particularly from about 25% to about 50% of component A). The oral dosage compositions further comprise about 50 to about 95% of component B), and more particularly, from about 50 to about 75%.

Tablets can be compressed, tablet triturates, enteric-coated, sugar-coated, film-coated, or multiple-compressed. Tablets typically comprise component A, and component B a carrier comprising ingredients selected from the group consisting of a) diluents, b) lubricants, c) binders, d) disintegrants, e) colorants, f) flavors, g) sweeteners, k) glidants, and combinations thereof. Specific diluents include calcium carbonate, sodium carbonate, mannitol, lactose and cellulose. Specific binders include starch, gelatin, and sucrose. Specific disintegrants include alginic acid and croscarmelose. Specific lubricants include magnesium stearate, stearic acid, and talc. Specific colorants are the FD&C dyes, which can be added for appearance. Chewable tablets preferably contain g) sweeteners such as aspartame and saccharin, or f) flavors such as menthol, peppermint, fruit flavors, or a combination thereof.

Capsules (including implants, time release and sustained release formulations) typically comprise component A, and a carrier comprising one or more a) diluents disclosed above in a capsule comprising gelatin. Granules typically comprise component A, and preferably further comprise k) glidants such as silicon dioxide to improve flow characteristics. Implants can be of the biodegradable or the non-biodegradable type. Implants may be prepared using any known biocompatible formulation.

The selection of ingredients in the carrier for oral compositions depends on secondary considerations like taste, cost, and shelf stability, which are not critical for the purposes of this disclosure. One skilled in the art would know how to select appropriate ingredients without undue experimentation.

The solid compositions may also be coated by conventional methods, typically with pH or time-dependent coatings, such that component A is released in the gastrointestinal tract in the vicinity of the desired application, or at various points and times to extend the desired action. The coatings typically comprise one or more components selected from the group consisting of cellulose acetate phthalate, polyvinyl acetate phthalate, hydroxypropyl methyl cellulose phthalate, ethyl cellulose, EUDRAGIT® coatings (available from Rohm & Haas G.M.B.H. of Darmstadt, Germany), waxes and shellac.

Compositions for oral administration can also have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically comprise component A and component B, namely, a carrier comprising ingredients selected from the group consisting of a) diluents, e) colorants, f) flavors, g) sweeteners, j) preservatives, m) solvents, n) suspending agents, and o) surfactants. Peroral liquid compositions preferably comprise one or more ingredients selected from the group consisting of e) colorants, f) flavors, and g) sweeteners.

Other compositions useful for attaining systemic delivery of the subject compounds include injection, sublingual, buccal and nasal dosage forms. Such compositions typically comprise one or more of soluble filler substances such as a) diluents including sucrose, sorbitol and mannitol; and c) binders such as acacia, microcrystalline cellulose, carboxymethyl cellulose, and hydroxypropyl methylcellulose. Such compositions may further comprise b) lubricants, e) colorants, f) flavors, g) sweeteners, h) antioxidants, and k) glidants.

In one embodiment of the disclosure, the compounds of the present disclosure are topically administered. Topical compositions that can be applied locally to the eye may be in any form known in the art, non-limiting Examples of which include solids, gelable drops, sprays, ointments, or a sustained or non-sustained release unit placed in the conjunctival cul-du-sac of the eye or another appropriate location.

Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions comprise: component A, the compounds described above, and component B, a carrier. The carrier of the topical composition preferably aids penetration of the compounds into the eye. Component B may further comprise one or more optional components.

A therapeutically effective amount of a compound according to the present disclosure will vary with the particular condition being treated, the age and physical condition of the patient being treated, the severity of the condition, the duration of treatment, the nature of concurrent therapy, the route of administration, the particular pharmaceutically-acceptable carrier utilized, and like factors within the knowledge and expertise of the attending physician. For example, a therapeutically effective amount of the compounds of the present disclosure for systemic administration is from about 0.01 to about 1000 μg/kg body weight, preferably from about 0.1 to about 100 μg/kg per body weight, most preferably form about 1 to about 50 μg/kg body weight per day. The transdermal dosages will be designed to attain similar serum or plasma levels, based upon techniques known to those skilled in the art of pharmacokinetics and transdermal formulations. Plasma levels for systemic administration are expected to be in the range of 0.01 to 100 ng/ml, more preferably from 0.05 to 50 ng/ml and most preferably from 0.1 to 10 ng/ml. While these dosages are based upon a daily administration rate, the compounds of the present disclosure may also be administered at other intervals, such as twice per day, twice weekly, once weekly, or once a month. One of ordinary skill in the art would be able to calculate suitable therapeutically effective amounts for other intervals of administration.

The compounds of the present disclosure are useful in a method of reducing or decreasing intraocular pressure. The compounds of the present disclosure may be administered to a subject in need of treatment in a amount therapeutically effective to reduce intraocular pressure. Thus, these compounds are useful in the treatment of glaucoma. The preferred route of administration for treating glaucoma is topically.

The exact amounts of each component in the topical composition depend on various factors. The amount of component A added to the topical composition is dependent on the IC₅₀ of component A, typically expressed in nanomolar (nM) units. For example, if the IC₅₀ of the medicament is 1 nM, the amount of component A will be from about 0.001 to about 0.3%. If the IC₅₀ of the medicament is 10 nM, the amount of component A) will be from about 0.01 to about 1%. If the IC₅₀ of the medicament is 100 nM, the amount of component A will be from about 0.1 to about 10%. If the amount of component A is outside the ranges specified above (i.e., lower), efficacy of the treatment may be reduced. One skilled in the art understands how to calculate and understand an IC₅₀. The remainder of the composition, up to 100%, is component B.

The amount of the carrier employed in conjunction with component A is sufficient to provide a practical quantity of composition for administration per unit dose of the medicament. Techniques and compositions for making dosage forms useful in the methods of this disclosure are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

Component B may comprise a single ingredient or a combination of two or more ingredients. In the topical compositions, component B comprises a topical carrier. Suitable topical carriers comprise one or more ingredients selected from the group consisting of phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols and symmetrical alcohols.

The carrier of the topical composition may further comprise one or more ingredients selected from the group consisting of q) emollients, r) propellants, s) solvents, t) humectants, u) thickeners, v) powders, w) fragrances, x) pigments, and y) preservatives.
Ingredient q) is an emollient. The amount of ingredient q) in a skin-based topical composition is typically about 5 to about 95%. Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane.
Ingredient r) is a propellant. The amount of ingredient r) in the topical composition is typically about 0 to about 95%. Suitable propellants include propane, butane, isobutane, dimethyl ether, carbon dioxide, nitrous oxide, and combinations thereof.
Ingredient s) is a solvent. The amount of ingredient s) in the topical composition is typically about 0 to about 95%. Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols.
Ingredient t) is a humectant. The amount of ingredient t) in the topical composition is typically 0 to 95%. Suitable humectants include glycerin, sorbitol, sodium 2-pyrrolidone-5-carboxylate, soluble collagen, dibutyl phthalate, gelatin, and combinations thereof. Specific humectants include glycerin.
Ingredient u) is a thickener. The amount of ingredient u) in the topical composition is typically about 0 to about 95%.
Ingredient v) is a powder. The amount of ingredient v) in the topical composition is typically 0 to 95%. Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. For ocular applications, specific powders include beta-cyclodextrin, hydroxypropyl cyclodextrin, and sodium polyacrylate. For gel dosing ocular formulations, sodium polyacrylate may be used.
Ingredient w) is a fragrance. The amount of ingredient w) in the topical composition is typically about 0 to about 0.5%, particularly, about 0.001 to about 0.1%. For ocular applications a fragrance is not typically used.
Ingredient x) is a pigment. Suitable pigments for skin applications include inorganic pigments, organic lake pigments, pearlescent pigments, and mixtures thereof. Inorganic pigments useful in this disclosure include those selected from the group consisting of rutile or anatase titanium dioxide, coded in the Color Index under the reference CI 77,891; black, yellow, red and brown iron oxides, coded under references CI 77,499, 77,492 and, 77,491; manganese violet (CI 77,742); ultramarine blue (CI 77,007); chromium oxide (CI 77,288); chromium hydrate (CI 77,289); and ferric blue (CI 77,510) and mixtures thereof.

The organic pigments and lakes useful in this disclosure include those selected from the group consisting of D&C Red No. 19 (CI 45,170), D&C Red No. 9 (CI 15,585), D&C Red No. 21 (CI 45,380), D&C Orange No. 4 (CI 15,510), D&C Orange No. 5 (CI 45,370), D&C Red No. 27 (CI 45,410), D&C Red No. 13 (CI 15,630), D&C Red No. 7 (CI 15,850), D&C Red No. 6 (CI 15,850), D&C Yellow No. 5 (CI 19,140), D&C Red No. 36 (CI 12,085), D&C Orange No. 10 (CI 45,425), D&C Yellow No. 6 (CI 15,985), D&C Red No. 30 (CI 73,360), D&C Red No. 3 (CI 45,430), the dye or lakes based on Cochineal Carmine (CI 75,570) and mixtures thereof.

The pearlescent pigments useful in this disclosure include those selected from the group consisting of the white pearlescent pigments such as mica coated with titanium oxide, bismuth oxychloride, colored pearlescent pigments such as titanium mica with iron oxides, titanium mica with ferric blue, chromium oxide and the like, titanium mica with an organic pigment of the above-mentioned type as well as those based on bismuth oxychloride and mixtures thereof. The amount of pigment in the topical composition is typically about 0 to about 10%. For ocular applications a pigment is generally not used.

In a particularly preferred embodiment of the disclosure, topical pharmaceutical compositions for ocular administration are prepared typically comprising component A and B (a carrier), such as purified water, and one or more ingredients selected from the group consisting of y) sugars or sugar alcohols such as dextrans, particularly mannitol and dextran 70, z) cellulose or a derivative thereof, aa) a salt, bb) disodium EDTA (Edetate disodium), and cc) a pH adjusting additive.
Examples of z) cellulose derivatives suitable for use in the topical pharmaceutical composition for ocular administration include sodium carboxymethylcellulose, ethylcellulose, methylcellulose, and hydroxypropyl-methylcellulose, particularly, hydroxypropyl-methylcellulose.
Examples of aa) salts suitable for use in the topical pharmaceutical composition for ocular administration include mono-, di- and trisodium phosphate, sodium chloride, potassium chloride, and combinations thereof.
Examples of cc) pH adjusting additives include HCl or NaOH in amounts sufficient to adjust the pH of the topical pharmaceutical composition for ocular administration to the range of 4.5-7.5 pH units.

### Kits

Component A may be included in kits comprising a compound as described herein, a systemic or topical composition described above, or both; and information, instructions, or both that use of the kit will provide treatment for cosmetic and medical conditions in mammals (particularly humans). The information and instructions may be in the form of words, pictures, or both, and the like. In addition or in the alternative, the kit may comprise the medicament, a composition, or both; and information, instructions, or both, regarding methods of application of medicament, or of composition, preferably with the benefit of treating or preventing cosmetic and medical conditions in mammals (e.g., humans).

In some embodiments, provided herein are packaged dosage forms, comprising a container holding a therapeutically effective amount of a compound provided herein or its salt, or a composition provided herein, and instructions for using the dosage form in accordance with one or more of the methods provided herein.

The dosage forms and associated materials can be finished as a commercial product by the usual steps performed in the present field, for example by appropriate sterilization and packaging steps. For example, the material can be treated by UV/vis irradiation (200-500 nm), for example using photo-initiators with different absorption wavelengths (for example, Irgacure 184, 2959), preferably water-soluble initiators (for example, Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (for example, by the addition of specific product information leaflets) into suitable containers (boxes, etc.).

According to further embodiments, the dosage forms can also be provided in kit form combined with other components necessary for administration of the material to the patient. For example, disclosed kits, such as for use in the treatments described herein, can further comprise, for example, administration materials.

The kits may be designed in various forms based on the specific deficiencies they are designed to treat.

The compounds or compositions herein may be prepared and placed in a container for storage at ambient or elevated temperature. When the compounds or compositions provided herein are stored in a polyolefin plastic container as compared to, for example, a polyvinyl chloride plastic container, discoloration of the compounds or compositions may be reduced. Without wishing to be bound by theory, the container may reduce exposure of the container's contents to electromagnetic radiation, whether visible light (for example, having a wavelength of about 380-780 nm) or ultraviolet (UV) light (for example, having a wavelength of about 190-320 nm (UV B light) or about 320-380 nm (UV A light)). Some containers also include the capacity to reduce adherence or adsorption of an active ingredient to the surface of the container, which could effectively dilute the concentration of active compound in the contained solution. Some containers also include the capacity to reduce exposure of the container's contents to infrared light, or a second component with such a capacity. Some containers further include the capacity to reduce the exposure of the container's contents to heat or humidity. The containers that may be used include those made from a polyolefin such as polyethylene, polypropylene, polyethylene terephthalate, polycarbonate, polymethylpentene, polybutene, a polyester, or any combination thereof. In some embodiments, the container is one made from a polymer including polyethylene, polypropylene, or a combination thereof. In some embodiments, the container is a glass container. The container may further be disposed within a second container, for example, a paper container, cardboard container, paperboard container, metallic film container, or foil container, or a combination thereof, to further reduce exposure of the container's contents to UV, visible, or infrared light. Articles of manufacture benefiting from reduced discoloration, decomposition, or both during storage, include the compounds described herein whether in neutral form, as a salt, or a composition thereof. The compounds or compositions provided herein may need storage lasting up to, or longer than, three months; in some cases up to, or longer than one year. The containers may be in any form suitable to contain the contents—for example, a bag, a bottle, or a box.

The disclosure will be further explained by the following illustrative Examples that are to be considered to be non-limiting.

### EXAMPLES

### Example 1: Preparation of E3

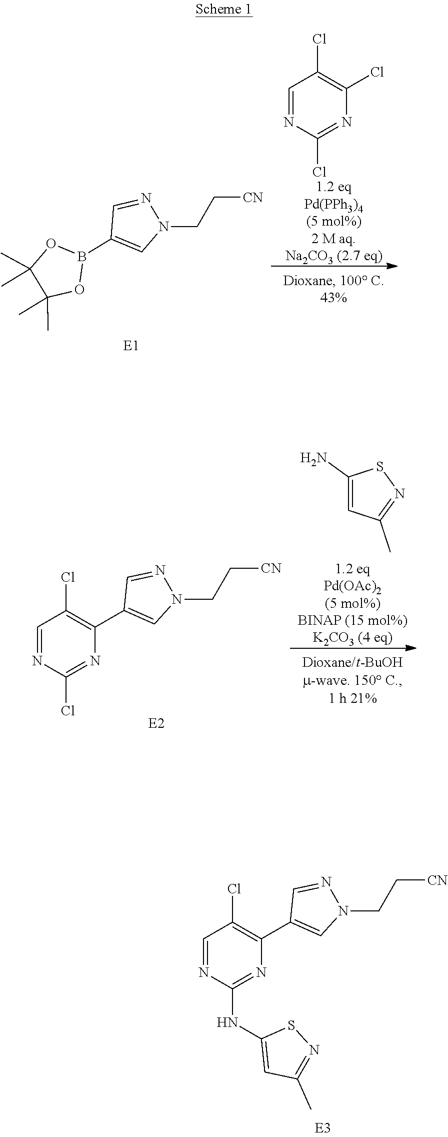

Preparation of 3-(4-(2,5-dichloropyrimidin-4-yl)-1H-pyrazol-1-yl)propanenitrile (E2): To a dry vial was added 3-(4-(4,4,5,5-tetramethyl-1,3,2-dioxaborolan-2-yl)-1H-pyrazol-1-yl)propanenitrile (E1, 1 eq), 2,4,5-trichloropyrimidine (1.2 eq), tetrakis(triphenylphosphine)palladium (0) (5 mol %), and 2 mL aqueous sodium carbonate. The solution was diluted with 1,4-dioxane, then heated to 100° C. for 6 h. The solution was cooled to room temperature, then buffered to pH=7. The reaction mixture was extracted with ethyl acetate (3×). The combined organics were dried over magnesium sulfate, then filtered and evaporated. Column chromatography (hexanes:ethyl acetate) afforded 3-(4-(2,5-dichloropyrimidin-4-yl)-1H-pyrazol-1-yl)propanenitrile (E2, 48%), which was carried forward without any further purification.

Preparation of 3-(4-(5-chloro-2-((3-methylisothiazol-5-yl)amino)pyrimidin-4-yl)-1H-pyrazol-1-yl)propanenitrile (E3). To a dry microwave vial was added 3-(4-(2,5-dichloropyrimidin-4-yl)-1H-pyrazol-1-yl)propanenitrile (E2) (1 eq), 3-Methyl-1,2-thiazol-5-amine (1.2 eq), palladium (II) acetate (5 mol %), BINAP (15 mol %), potassium carbonate (4 eq), 1,4-dioxane, and tert-butyl alcohol. The microwave vial was capped under a blanket of nitrogen. The reaction mixture was irradiated for 1 h at 150° C. The reaction mixture was filtered through a syringe filter, then buffered to pH=7. The aqueous layer was extracted first with 3:1 DCM: IPA, then ethyl acetate (3×). The combined organics were dried over magnesium sulfate, then filtered and evaporated. Column chromatography (hexanes:ethyl acetate, then dichloromethane:methanol) to afford 3-(4-(5-chloro-2-((3-methylisothiazol-5-yl)amino)pyrimidin-4-yl)-1H-pyrazol-1-yl)propanenitrile (E3, 35%).

### Example 2: Preparation of E7

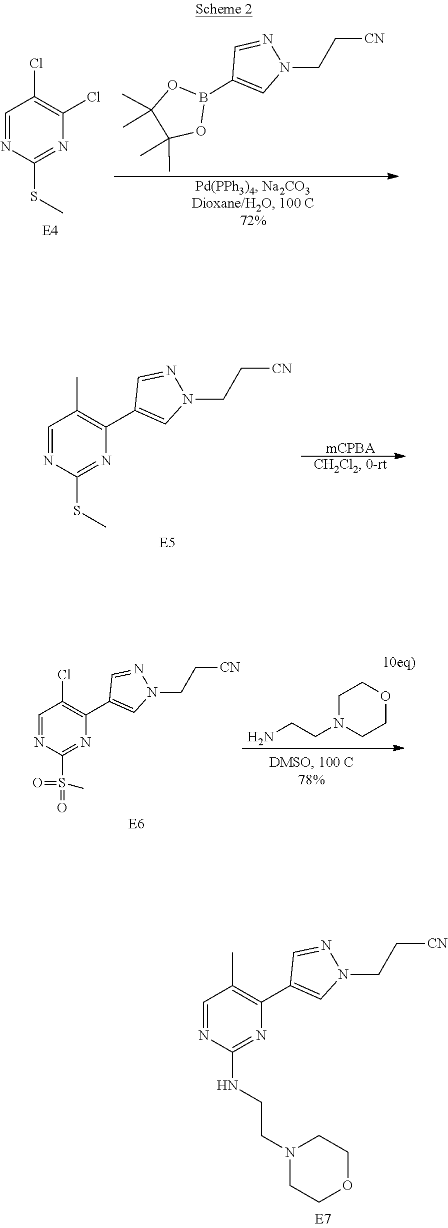

Preparation of 3-(4-(5-methyl-2-(methylthio)pyrimidin-4-yl)-1H-pyrazol-1-yl)propanenitrile (E5). To 4-chloro-5-methyl-2-(methylthio) pyrimidine (E4) dissolved in dioxane and water was added 3-(4-(4,4,5,5-tetramethyl-1,3,2-dioxaborolan-2-yl)-1H-pyrazol-1-yl)propanenitrile, Na₂CO₃ and Pd(PPh₃)₄ and the solution was heated to 98-100° C. for 4-5 h. The mixture was cooled and poured into EtOAc and water and extracted with EtOAc, dried (Na₂SO₄), filtered and evaporated. Column Chromatography (Hexanes-Ethyl Acetate) gave 3-(4-(5-methyl-2-(methylthio)pyrimidin-4-yl)-1H-pyrazol-1-yl)propanenitrile (E5, 72%).

Preparation of 3-(4-(5-methyl-2-(methylsulfonyl)pyrimidin-4-yl)-1H-pyrazol-1-yl)propanenitrile (E6). To 3-(4-(5-methyl-2-(methylthio)pyrimidin-4-yl)-1H-pyrazol-1-yl)propanenitrile (E5) in CH₂Cl₂ cooled to 0° C. was added MCPBA and the solution was allowed to slowly warm to room temperature for 4-5 hours. The mixture was poured into CH₂Cl₂ (or EtOAc) and NaHCO₃(sat), extracted, dried (Na₂SO₄), filtered and evaporated. Column chromatography (CH₂Cl₂-MeOH) gave pure 3-(4-(5-methyl-2-(methylsulfonyl)pyrimidin-4-yl)-1H-pyrazol-1-yl)propanenitrile (E6, 49%).

Preparation of 3-(4-(5-methyl-2-((2-morpholinoethyl)amino)pyrimidin-4-yl)-1H-pyrazol-1-yl)propanenitrile (E7). To 3-(4-(5-methyl-2-(methylsulfonyl)pyrimidin-4-yl)-1H-pyrazol-1-yl)propanenitrile (E6) in DMSO was added 2-morpholinoethan-1-amine (10 eq) and the mixture was heated to 100° C. for 4-12 h. The solution was cooled to room temperature and poured into CH₂Cl₂ and NaHCO₃ (sat) and extracted with CH₂Cl₂, dried (Na₂SO₄), filtered and evaporated. Column chromatography (CH₂Cl₂-MeOH) have pure of 3-(4-(5-methyl-2-((2-morpholinoethyl)amino)pyrimidin-4-yl)-1H-pyrazol-1-yl)propanenitrile (E7, 76%).

### Example 3: Preparation of E10

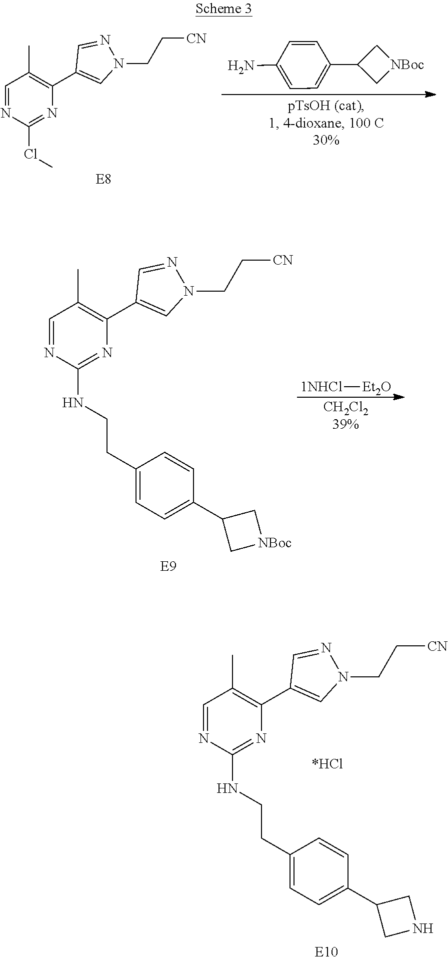

Preparation of tert-butyl 3-(4-((4-(1-(2-cyanoethyl)-1H-pyrazol-4-yl)-5-methylpyrimidin-2-yl)amino)phenyl)azetidine-1-carboxylate (E9). To 3-(4-(2-chloro-5-methylpyrimidin-4-yl)-1H-pyrazol-1-yl)propanenitrile (E8) in 1,4-dioxane was added tert-butyl 3-(4-aminophenyl)azetidine-1-carboxylate and pTsOH and the mixture was heated in a pressure tube to 100° C. for 12 h. The solution was cooled to room temperature, poured into EtOAc and NaHCO₃(sat) and extracted with EtOAc, dried (Na₂SO₄), filtered and evaporated. Column chromatography (CH₂Cl₂-MeOH and or Hexanes-Ethyl acetate) gave tert-butyl3-(4-((4-(1-(2-cyanoethyl)-1H-pyrazol-4-yl)-5-methylpyrimidin-2yl)amino)phenyl)azetidine-1-carboxylate (E9, 30%).

Preparation of 3-(4-(2-((4-(azetidin-3-yl)phenyl)amino)-5-methylpyrimidin-4-yl)-1H-pyrazol-1-yl)propanenitrile hydrochloride (E10). To tert-butyl 3-(4-((4-(1-(2-cyanoethyl)-1H-pyrazol-4-yl)-5-methylpyrimidin-2-yl)amino)phenyl)azetidine-1-carboxylate (E9) in CH₂Cl₂ was added (HCl-ether (1N) and the solution was stirred for 12 hours at 30° C. The solvents were evaporated and column chromatography (CH₂Cl₂-MeOH and CH₂Cl₂-EtOH) gave 3-(4-(2-((4-(azetidin-3-yl)phenyl)amino)-5-methylpyrimidin-4-yl)-1H-pyrazol-1-yl)propanenitrile hydrochloride (E10).

Procedures analogous to those set forth above for Schemes 1, 2, and 3, with substitution of the appropriate starting materials, were used to prepare additional compounds provided herein.

### Example 4: Preparation of E12

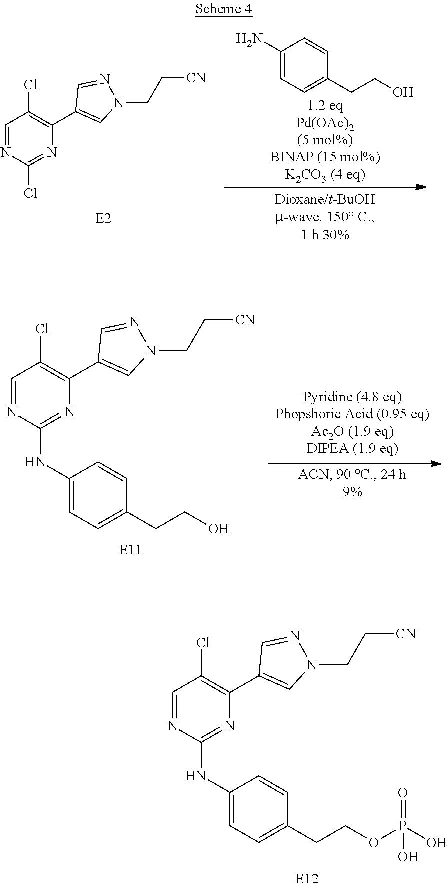 [SMILES: N#CCCn1cc(-c2nc(Cl)ncc2Cl)cn1 ; Nc1ccc(CCO)cc1 ; N#CCCn1cc(-c2nc(Nc3ccc(CCO)cc3)ncc2Cl)cn1 ; N#CCCn1cc(-c2nc(Nc3ccc(CCOP(=O)(O)O)cc3)ncc2Cl)cn1]

Preparation of 5-chloro-2-((4-(2-hydroxyethyl)phenyl)amino)pyrimidin-4-yl)-1H-pyrazol-1-yl)propanenitrile (E11). To a dry microwave vial was added 3-(4-(2,5-dichloropyrimidin-4-yl)-1H-pyrazol-1-yl)propanenitrile (E2) (1 eq), 2-(4-aminophenyl) ethan-1-ol (1.2 eq), palladium (II) acetate (5 mol %), BINAP (15 mol %), potassium carbonate (4 eq), 1,4-dioxane, and tert-butyl alcohol. The microwave vial was capped under a blanket of nitrogen. The reaction mixture was irradiated for 1 h at 150° C. The reaction mixture was filtered through a syringe filter, then buffered to pH=7. The aqueous layer was extracted first with 3:1 DCM: IPA, then ethyl acetate (3×). The combined organics were dried over magnesium sulfate, then filtered and evaporated. Column chromatography (dichloromethane:methanol) to afford 5-chloro-2-((4-(2-hydroxyethyl)phenyl)amino)pyrimidin-4-yl)-1H-pyrazol-1-yl)propanenitrile (E11, 30%).

### Preparation of 5-chloro-4-(1-(2-cyanoethyl)-1H-pyrazol-4-yl)pyrimidin-2-yl)amino)phenethyl dihydrogen phosphate (E)

To a dry vial was added 5-chloro-2-((4-(2-hydroxyethyl)phenyl)amino)pyrimidin-4-yl)-1H-pyrazol-1-yl)propanenitrile (E2) (1 eq), phosphoric acid (0.95 eq), pyridine (4.8 eq), and acetonitrile. DIPEA (1.9 eq) was added and the reaction mixture stirred for 5 min. Acetic Anhydride (1.9 eq) was added and the reaction brought to 90° C. overnight. Cooled to room temperature and evaporated onto Celite. Column chromatography (dichloromethane:methanol) afforded 4-((5-chloro-4-(1-(2-cyanoethyl)-1H-pyrazol-4-yl)pyrimidin-2-yl)amino)phenethyl dihydrogen phosphate (E11, 30%).

### Example 5: Preparation of E16

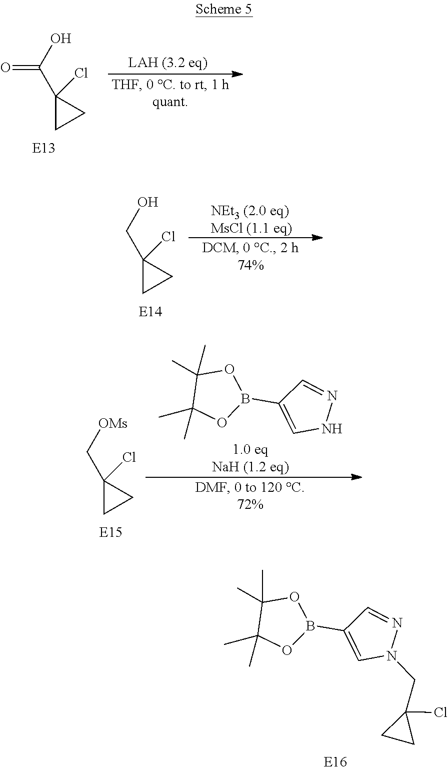 [SMILES: O=C(O)C1(Cl)CC1 ; OCC1(Cl)CC1 ; CS(=O)(=O)OCC1(Cl)CC1 ; CC1(C)OB(c2cn[nH]c2)OC1(C)C ; CC1(C)OB(c2cnn(CC3(Cl)CC3)c2)OC1(C)C]

Preparation of (1-chlorocyclopropyl) methanol (E14): To a dry roundbottomed flask was added 1-chlorocyclopropane-1-carboxylic acid (E13) (1 eq) in anhydrous THF. The solution was cooled to 0° C. in an ice/water bath. A solution of 1 M lithium aluminum hydride in THF (3.2 eq) was added dropwise. The solution was stirred at room temperature for 1 h. The solution was cooled to 0° C. in an ice/water bath. A solution of ethyl acetate/water (1:1) was added slowly due to generation of hydrogen gas. The resultant slurry was filtered through a plug of Celite and rinsed copiously with ethyl acetate and water. The organic layer was separated. The aqueous layer was extracted with ethyl acetate twice. The combined organics were dried over magnesium sulfate, then filtered and evaporated. Upon evaporation, (1-chlorocyclopropyl) methanol (E14) was carried forward without any further purification.

Preparation of (1-chlorocyclopropyl) methyl methanesulfonate (E15) To a dry roundbottomed flask was added (1-chlorocyclopropyl) methanol (E14) (1 eq) followed by anhydrous DCM and triethylamine (2 eq). The solution was cooled to 0° C. in an ice/water bath. Mesyl chloride (1.1 eq) was added dropwise. The solution was stirred at 0° C. for 2 h. The solution was quenched with saturated sodium bicarbonate and diluted with diethyl ether. The reaction mixture was stirred for 30 minutes at room temperature. The layers were separated and the aqueous layer extracted further with diethyl ether. The combined organics were dried over magnesium sulfate, then filtered and evaporated. Upon evaporation, (1-chlorocyclopropyl) methyl methanesulfonate (E15, 74%) was carried forward without any further purification.

Preparation of 1-((1-chlorocyclopropyl)methyl)-4-(4,4,5,5-tetramethyl-1,3,2-dioxaborolan-2-yl)-1H-pyrazole (E16) To a dry roundbottomed flask was added 4-(4,4,5,-tetramethyl-1,3,2-dioxaborolan-2-yl)-1H-pyrazole (1 eq) and anhydrous DMF. The solution was cooled to 0° C. in an ice/water bath. Sodium hydride (1.2 eq) was added all at once. The solution was stirred at 0° C. for 30 minutes. Then, (1-chlorocyclopropyl) methyl methanesulfonate (E15) was added in DMF via cannula. Following addition, the reaction mixture was heated to 120° C. overnight. The reaction mixture was cooled to room temperature. The reaction mixture was diluted with saturated sodium chloride, then extracted with ethyl acetate (3×). The combined organics were dried over magnesium sulfate, then filtered and evaporated. Column chromatography (hexanes:ethyl acetate) afforded 1-((1-chlorocyclopropyl)methyl)-4-(4,4,5,5-tetramethyl-1,3,2-dioxaborolan-2-yl)-1H-pyrazole (E16, 72%), which was carried forward without any further purification.

### Example 6: Preparation of E18

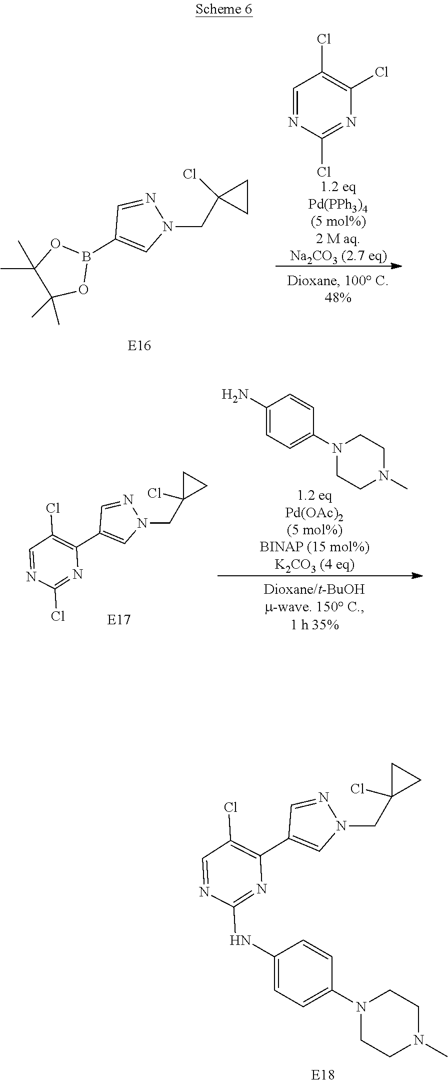 [SMILES: Clc1ncc(Cl)c(Cl)n1 ; CC1(C)OB(c2cnn(CC3(Cl)CC3)c2)OC1(C)C ; Clc1ncc(Cl)c(-c2cnn(CC3(Cl)CC3)c2)n1 ; CN1CCN(c2ccc(N)cc2)CC1 ; CN1CCN(c2ccc(Nc3ncc(Cl)c(-c4cnn(CC5(Cl)CC5)c4)n3)cc2)CC1]

Preparation of 2,5-dichloro-4-(1-((1-chlorocyclopropyl)methyl)-1H-pyrazol-4-yl)pyrimidine (E17). To a dry vial was added 1-((1-chlorocyclopropyl)methyl)-4-(4,4,5,5-tetramethyl-1,3,2-dioxaborolan-2-yl)-1H-pyrazole (E16), (1 eq), 2,4,5-trichloropyrimidine (1.2 eq), tetrakis(triphenylphosphine)palladium (0) (5 mol %), and 2 mL aqueous sodium carbonate. The solution was diluted with 1,4-dioxane, then heated to 100° C. for 6 h. The solution was cooled to room temperature, then buffered to pH=7. The reaction mixture was extracted with ethyl acetate (3×). The combined organics were dried over magnesium sulfate, then filtered and evaporated. Column chromatography (hexanes:ethyl acetate) afforded 2,5-dichloro-4-(1-((1-chlorocyclopropyl)methyl)-1H-pyrazol-4-yl)pyrimidine (E17, 48%) which was carried forward without any further purification.

Preparation of 5-chloro-4-(1-((1-chlorocyclopropyl)methyl)-1H-pyrazol-4-yl)-N-(4-(4-methylpiperazin-1-yl)phenyl)pyrimidin-2-amine (E18) To a dry microwave vial was added 2,5-dichloro-4-(1-((1-chlorocyclopropyl)methyl)-1H-pyrazol-4-yl)pyrimidine (E17) (1 eq), 4-Methyl-1-(4-aminophenyl) piperazine (1.2 eq), palladium (II) acetate (5 mol %), BINAP (15 mol %), potassium carbonate (4 eq), 1,4-dioxane, and tert-butyl alcohol. The microwave vial was capped under a blanket of nitrogen. The reaction mixture was irradiated for 1 h at 150° C. The reaction mixture was filtered through a syringe filter, then buffered to pH=7. The aqueous layer was extracted first with 3:1 DCM: IPA, then ethyl acetate (3×). The combined organics were dried over magnesium sulfate, then filtered and evaporated. Column chromatography (hexanes:ethyl acetate, then dichloromethane:methanol) to afford 5-chloro-4-(1-((1-chlorocyclopropyl)methyl)-1H-pyrazol-4-yl)-N-(4-(4-methylpiperazin-1-yl)phenyl)pyrimidin-2-amine (E18, 35%)

### Example 7: Preparation of E22

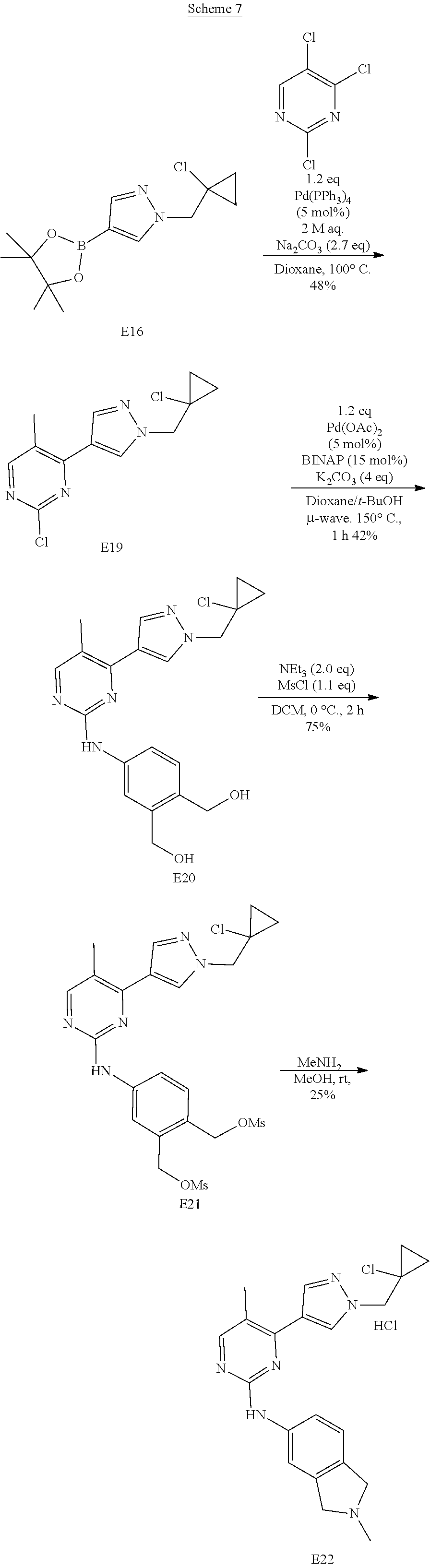

Preparation of 2-chloro-4-(1-((1-chlorocyclopropyl)methyl)-1H-pyrazol-4-yl)-5-methylpyrimidine (E19). To a dry vial was added 1-((1-chlorocyclopropyl)methyl)-4-(4,4,5,5-tetramethyl-1,3,2-dioxaborolan-2-yl)-1H-pyrazole (E16), (1 eq), 5-methyl-2,4-trichloropyrimidine (1.2 eq), tetrakis(triphenylphosphine)palladium (0) (5 mol %), and 2 mL aqueous sodium carbonate. The solution was diluted with 1,4-dioxane, then heated to 100° C. for 6 h. The solution was cooled to room temperature, then buffered to pH=7. The reaction mixture was extracted with ethyl acetate (3×). The combined organics were dried over magnesium sulfate, then filtered and evaporated. Column chromatography (hexanes:ethyl acetate) afforded 2-chloro-4-(1-((1-chlorocyclopropyl)methyl)-1H-pyrazol-4-yl)-5-methylpyrimidine (EX, 48%) which was carried forward without any further purification.

Preparation of (4-((4-1-((1-chlorocyclopropyl)methyl)-1H-pyrazol-4-yl)-5-methylpyrimidin-2-yl)amino)-1,2-phenylenedimethanol (E20) To a dry microwave vial was added 2-chloro-4-(1-((1-chlorocyclopropyl)methyl)-1H-pyrazol-4-yl)-5-methylpyrimidine (E19) (1 eq), (4-amino-1,2-phenylene) dimethanol (1.2 eq), palladium (II) acetate (5 mol %), BINAP (15 mol %), potassium carbonate (4 eq), 1,4-dioxane, and tert-butyl alcohol. The microwave vial was capped under a blanket of nitrogen. The reaction mixture was irradiated for 1 h at 150° C. The reaction mixture was filtered through a syringe filter, then buffered to pH=7. The aqueous layer was extracted first with 3:1 DCM: IPA, then ethyl acetate (3×). The combined organics were dried over magnesium sulfate, then filtered and evaporated. Column chromatography (hexanes:ethyl acetate, then dichloromethane:methanol) to afford (4-((4-1-((1-chlorocyclopropyl)methyl)-1H-pyrazol-4-yl)-5-methylpyrimidin-2-yl)amino)-1,2-phenylenedimethanol (E20, 42%)

Preparation of (4-((4-(1-((1-chlorocyclopropyl)methyl)-1H-pyrazol-4-yl)-5-methylpyrimidin-2-yl)amino)-1,2-phenylene)bis(methylene) dimethanesulfonate (E21) To a dry roundbottomed flask was added (4-((4-1-((1-chlorocyclopropyl)methyl)-1H-pyrazol-4-yl)-5-methylpyrimidin-2-yl)amino)-1,2-phenylenedimethanol (EX) (1 eq) followed by anhydrous DCM and triethylamine (2 eq). The solution was cooled to 0° C. in an ice/water bath. Mesyl chloride (1.1 eq) was added dropwise. The solution was stirred at 0° C. for 2 h. The solution was quenched with saturated sodium bicarbonate and diluted with DCM. The reaction mixture was stirred for 30 minutes at room temperature. The layers were separated and the aqueous layer extracted further with DCM. The combined organics were dried over magnesium sulfate, then filtered and evaporated. Upon evaporation, (4-((4-(1-((1-chlorocyclopropyl)methyl)-1H-pyrazol-4-yl)-5-methylpyrimidin-2-yl)amino)-1,2-phenylene)bis(methylene) dimethanesulfonate (E21, 75%) was carried forward without any further purification.

Preparation of N-4-(1-((1-chlorocyclopropyl)-1H-pyrazol-4-yl)-5-methylpyrimidin-2-yl)-2-methylisoindolin-5-amine (E22) To a dry roundbottomed flask was added (4-((4-(1-((1-chlorocyclopropyl)methyl)-1H-pyrazol-4-yl)-5-methylpyrimidin-2-yl)amino)-1,2-phenylene)bis(methylene) dimethanesulfonate (E21) (1 eq) in methanol followed by methylamine solution (2 M in methanol) (5 mL) at 0° C. in an ice/water bath. Brought to room temperature overnight. The reaction mixture was evaporated onto silica and was purified by column chromatography (hexanes:ethyl acetate, then dichloromethane:methanol) to afford N-4-(1-((1-chlorocyclopropyl)-1H-pyrazol-4-yl)-5-methylpyrimidin-2-yl)-2-methylisoindolin-5-amine. (E21). N-4-(1-((1-chlorocyclopropyl)-1H-pyrazol-4-yl)-5-methylpyrimidin-2-yl)-2-methylisoindolin-5-amine (E21) was suspended in 4 N HCl in dioxane followed by sonication for 5 min. After evaporation and drying under high vacuum, N-4-(1-((1-chlorocyclopropyl)-1H-pyrazol-4-yl)-5-methylpyrimidin-2-yl)-2-methylisoindolin-5-amine hydrochloride (E21) was obtained in 25% yield.

### Example 8: Preparation of E25 and E26

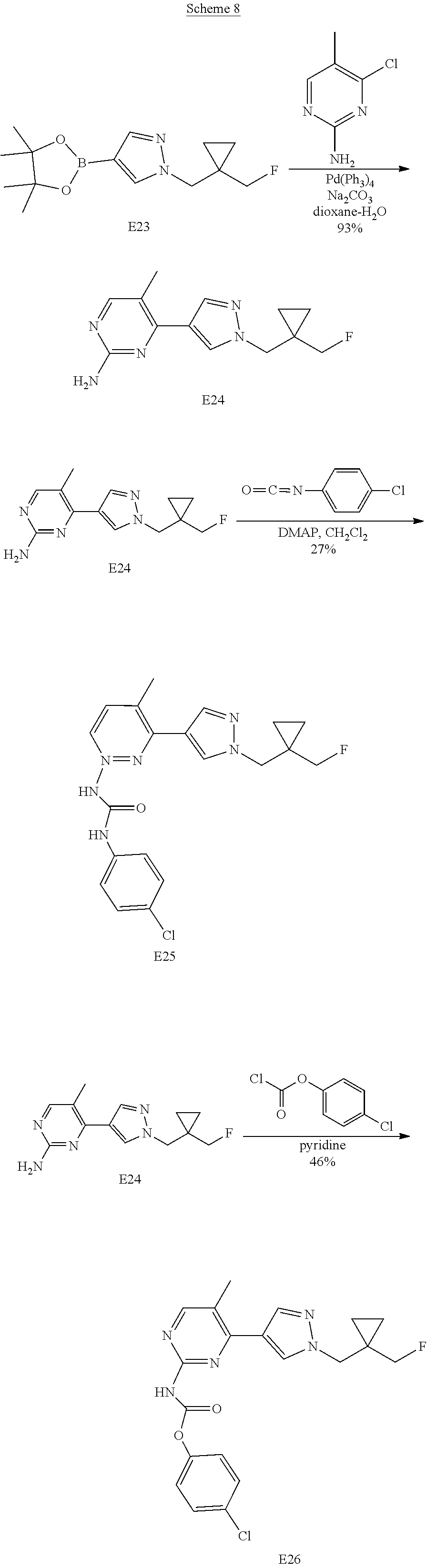

Preparation of 4-(1-((1-(fluoromethyl)cyclopropyl)methyl)-1H-pyrazol-4-yl)-5-methylpyrimidin-2-amine (E24). To 1-((1-(fluoromethyl)cyclopropyl)methyl)-4-(4,4,5,5-tetramethyl-1,3,2-dioxaborolan-2-yl)-1H-pyrazole (E23) in dioxane (3.7 mL) and water (1.2 mL) was added sodium carbonate (3 eq), 4-chloro-5-methylpyrimidin-2-amine (1 eq) and Pd(Ph₃)₄ (0.1 eq) and solution was stirred for 6 hours at 95-98° C. The mixture was cooled and poured into EtOAc and water. The aqueous was extracted with EtOAc and the organics were dried (Na₂SO₄), filtered and evaporation to give crude E24. Column chromatography (CH₂Cl₂-MeOH) gave pure 4-(1-((1-(fluoromethyl)cyclopropyl)methyl)-1H-pyrazol-4-yl)-5-methylpyrimidin-2-amine (E24, 93%).

Preparation of 1-(4-chlorophenyl)-3-(4-(1-((1-(fluoromethyl)cyclopropyl)methyl)-1H-pyrazol-4-yl)-5-methylpyrimidin-2-yl) urea (E25). To 4-(1-((1-(fluoromethyl)cyclopropyl)methyl)-1H-pyrazol-4-yl)-5-methylpyrimidin-2-amine (E24) in CH₂Cl₂ was added DMAP (0.05 eq) and 4-chlorophenylisocyanate (1.5 eq) and the solution was stirred at room temperature for 12 hours. The mixture was poured into EtOAc and NaHCO₃(sat) and further extracted with EtOAc, dried (Na₂SO₄), filtered and evaporated. Column chromatography (CH₂Cl₂-MeOH) gave To 1-(4-chlorophenyl)-3-(4-(1-((1-(fluoromethyl)cyclopropyl)methyl)-1H-pyrazol-4-yl)-5-methylpyrimidin-2-yl) urea (E25, 27%).

Preparation of 4-chlorophenyl (4-(1-((1-(fluoromethyl)cyclopropyl)methyl)-1H-pyrazol-4-yl)-5-methylpyrimidin-2-yl) carbamate (E26). To 4-(1-((1-(fluoromethyl)cyclopropyl)methyl)-1H-pyrazol-4-yl)-5-methylpyrimidin-2-amine (E24) in pyridine at 0° C. was added 4-chlorophenyl chloroformate (1.1 eq) and solution was slowly warmed to room temperature (3 hours). Then, the reaction as poured into EtOAc and NaHCO₃(sat) and further extracted with EtOAc, dried (Na₂SO₄), filtered and evaporated. Column chromatography (CH₂Cl₂-MeOH) gave pure 4-chlorophenyl (4-(1-((1-(fluoromethyl)cyclopropyl)methyl)-1H-pyrazol-4-yl)-5-methylpyrimidin-2-yl) carbamate (E26, 46%).

### Example 9: JAK Assays

All compounds were initially prepared as 10 mM stocks in anhydrous dimethylsulfoxide (DMSO). A 20 μL aliquot of the 10 mM solutions was transferred to individual wells in column 1 of a 96-well polypropylene microtiter plate (Corning #3363) and diluted with DMSO to give a final compound concentration of 4 mM. Test compounds were then serially diluted 1:5 in DMSO for an 11-point concentration response and further diluted in the assay buffer bringing all compound concentrations to a final range of 100 μM to 10 pM in 2.5% DMSO. The assay was performed in white 96-well, flat-bottom, half-area, non-binding assay plate (Corning #3642) in assay buffer consisting of 20 mM HEPES (pH 7.5), 10 mM MgCl₂*6H₂O, 100 μM sodium orthovanadate, 0.05% CHAPS and 0.1% bovine serum albumin. A 10 μL aliquot of compound from each well of the intermediate dilution plate and 20 μL of a 2×JAK substrate/enzyme solution containing acceptor substrate (800 nM Abl peptide), JAK enzyme (10 nM JAK1, JAK2, JAK3, or TYK2), and 1,4-Dithiothreitol (DTT, 2 μM) were added to all wells. The reaction was initiated by the addition of 10 μL of 4× stock solution ATP (2 μM). Reactions were thoroughly mixed manually, covered and allowed to incubate at room temperature for 75 min. Protein kinase activity was quantitated using Promega's KINASE-GLO™ luminescent Kinase Assay Kit according to the manufacturer's directions. ATP concentrations remaining in Test wells following the termination of the enzymatic reaction were compared against control wells containing equivalent amounts of DMSO containing no inhibitor (CTRL). ATP concentrations in both Test wells and CTRL wells were normalized against background (BKG) ATP concentrations in wells containing concentrations of inhibitor that completely inhibited the protein kinase under investigation (i.e. a concentration that prevented any consumption of ATP over the course of the incubation). Percent of Control (POC) values were determined for each concentration of compound tested according to the equation:

$$POC = ( ( Test well value - BKG ) / ( CTRL - BKG ) ) * 100.$$

IC₅₀ values were calculated using the following 4-parameter logistic curve-fitting algorithm:

$$f ( x ) = ( A + ( ( B - A ) / ( 1 + ( ( x / C ) \hat{} D ) ) ) ) .$$

IC₅₀ values were converted to Kᵢ values using the Cheng-Prusoff Equation:

$${K}_{i} = {IC}_{50} / ( 1 + ( [ ATP ] / Km ATP ] ) ) .$$

### Example 10. Reference Example—Pharmacological Activity for Glaucoma Assay

Pharmacological activity for glaucoma can be demonstrated using assays designed to test the ability of the subject compounds to decrease intraocular pressure. Examples of such assays are described in the following reference, incorporated herein by reference: C. Liljebris, G. Selen, B. Resul, J. Sternschantz, and U. Hacksell, “Derivatives of 17-phenyl-18, 19, 20-trinorprostaglandin F2a Isopropyl Ester: Potential Anti-glaucoma Agents,” Journal of Medicinal Chemistry 1995, 38 (2): 289-304.

While the disclosure has been described in detail and with reference to specific embodiments thereof, it will be apparent to one skilled in the art that various changes and modifications can be made without departing from the spirit and scope of the disclosure.

The preceding disclosures are illustrative embodiments. It should be appreciated by those of skill in the art that the devices, techniques and methods disclosed herein elucidate representative embodiments that function well in the practice of the present disclosure. However, those of skill in the art should, in light of the present disclosure, appreciate that many changes can be made in the specific embodiments that are disclosed and still obtain a like or similar result without departing from the spirit and scope of the disclosure.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the disclosure provided herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this disclosure are described herein, including the best mode known to the inventors for carrying out the disclosure. Of course, variations on those preferred embodiments will become apparent to those of ordinary skill in the art upon reading the foregoing description. The inventor expects those of ordinary skill in the art to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

Further, it is to be understood that the embodiments of the disclosure provided herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

We claim:

1. A compound of the formula: 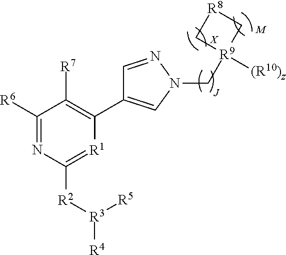
or a pharmaceutically acceptable salt thereof,
wherein
R¹ is N, CH, CF, CCl, or CBr;
R² is NH, 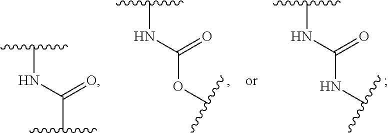 [SMILES: *NC(*)=O ; *NC(=O)O* ; *NC(=O)N*]
R³ is C₆₋₁₀ aryl or C₂₋₁₀ heteroaryl;
R⁴ is H, C₁-4 alkyl, C₁₋₄ haloalkyl, F, Cl, Br, I, O(C₁₋₄ alkyl), C₁₋₄ alkyl-OH, CH₂NH₂, CH₂N(C₁₋₄ alkyl)(C₁₋₄ alkyl), Boc, C(O)N(H)(C₁₋₄ alkyl), CH₂N(H)Boc, C(O)O(C₁₋₄ alkyl), C₂₋₈ heterocycloalkyl, CH₂—(C₂₋₈ heterocycloalkyl), (C₂₋₈ heterocycloalkyl)-CH₃, CH₂—(C₂₋₈ heterocycloalkyl)-CH₃, C(O)OH, S(O₂)(C₁₋₄ alkyl), C(O)NH₂, C₁₋₆ heteroalkyl, CH₂OC(O)—(C₆₋₁₀ aryl), (C₂₋₈ heterocycloalkyl)-Boc, (C₁₋₆ heteroalkyl)-(C₂₋₈ heterocycloalkyl)-(C₁₋₄ alkyl), CH₂OC(O)(C₁₋₆ heteroalkyl), CH₂OC(O)CH₂CH₂—(C₂₋₈ heterocycloalkyl), C(O)N(C₁₋₄ alkyl)(C₁₋₄ alkyl), CH₂N(H)C(O)C(CH₃)N(H)Boc, CH₂N(H)C(O)-pyrrolidinyl-Boc, CH₂N(H)C(O)C(CH₃)NH₂, CH₂N(H)C(O)-pyrrolidinyl, CH₂N(H)Boc, CH₂N(H)C(O)CH₂N(CH₃)₂, CH₂N(H)C(O)C(N(H)Boc)CH₂CH₂CH₂-guanidinyl(Boc)₂, CH₂N(H)C(O)C(NH₂)CH₂CH₂CH₂-guanidinyl, (C₂₋₈ heterocycloalkyl)-N(C₁₋₄ alkyl)(C₁₋₄ alkyl), or a C₅₋₁₀ spiroheterocycloalkyl;
R⁵ is H, F, Cl, Br, I, C₁₋₄ alkyl, C₁₋₄ haloalkyl, C₁₋₄ O-alkyll, C₁₋₄ alkyl-OH, ═O, or C(O)O(C₁₋₄ alkyl);
or R⁴ and R⁵ together form —O—N═C(H)—, —C(H₂)—N(H)—C(O)—, —C(O)—N(CH₃)—C(O)—, —C(H₂)—N(CH₃)—C(H₂)—, or ═C(H)—C(H)═N—;
R⁶ is H and R⁷ is H, F, Cl, Br, I, C₁₋₄ alkyl, C₁₋₄ haloalkyl, C₁₋₄ O-alkyll, or C₃₋₆ cycloalkyl;
or R⁶ and R⁷ together form a C₂₋₅ alkylene or a C₂₋₅ heteroalkylene;
R⁸ is a bond, NH, O, CH₂, N(Boc), N(CH₂F), N(CHF₂), N(CF₃), N(CH₂CH₂F), N(CH₂CHF₂), N(CH₂CF₃), N(CH₂CN), or N(CH₂CH₂CN);
R⁹ is N and Z is 0;
or R⁹ is C and Z is 1;
R¹⁰ is H, CH₂F, CHF₂, CF₃, CH₂Cl, CHCl₂, CCl₃, CH₂Br, CH₂I, F, Cl, Br, I, C₁₋₄ O-alkyll, C₁₋₃ alkylene-OH, CN, CH₂CN, CH₂CH₂CN, C(O)OH, OH, NH₂, N(H)CH₃, N(CH₃)₂, CH₂NH₂, CH₂N(H)CH₃, CH₂N(CH₃)₂, or N(Boc)CH₃;
J is 0, 1, or 2;
X is 0, 1, or 2;
M is 1 or 2; and
each hydrogen, independently, may be replaced with deuterium.

2. The compound of claim 1, wherein R¹ is N, CH, or CF.

3. The compound of claim 1, wherein R⁶ is H and R⁷ is H, F, Cl, CH₃, CH₂CH₃, OCH₃, or cyclopropyl.

4. The compound of claim 1, wherein R⁸ is a bond, NH, O, CH₂, N(CH₂F), or N(CH₂CN).

5. The compound of claim 1, wherein R⁹ is C and Z is 1.

6. The compound of claim 1, wherein R¹⁰ is H, CH₂F, CHF₂, F, Cl, CH₂OH, CN, CH₂CN, C(O)OH, N(H)CH₃, or CH₂N(CH₃)₂.

7. The compound of claim 1, wherein R⁴ is H, CH₃, CH₂NH₂, CH₂N(CH₃)₂, BOC, C(O)N(H)CH₃, CH₂N(H)Boc, piperazinyl, C(O)OCH₃, piperazinyl-CH₃, C(O)OH, S(O₂)CH₃, C(O)NH₂, morpholinyl, CH₂OH, Cl, OCH₃, CH₂-morpholinyl, CH₂OCH₃, CH₂-piperazinyl-CH₃, CH₂CH₂N(CH₃)₂, CH₂OC(O)-phenyl, CH₂N(H)CH₃, piperazinyl-Boc, CH₂OCH₂CH₂OCH₃, CH₂OC(O)CH₂CH₂-piperazynyl-CH₃, CH₂OC(O)CH₂CH₂N(CH₃)₂, CH₂OC(O)CH₂CH₂-morpholinyl, C(O)N(CH₃)₂, CH₂N(H)C(O)C(CH₃)N(H)Boc, CH₂N(H)C(O)-pyrrolidinyl-Boc, CH₂N(H)C(O)C(CH₃)NH₂, CH₂N(H)C(O)-pyrrolidinyl, CH₂N(H)Boc, CH₂N(H)C(O)CH₂N(CH₃)₂, CH₂N(H)C(O)C(N(H)Boc)CH₂CH₂CH₂-guanidinyl(Boc)₂, CH₂N(H)C(O)C(NH₂)CH₂CH₂CH₂-guanidinyl, CF₃, OCH₂CH₂N(CH₃)₂, OCH₂CH₂OCH₃, azetidinyl-N(CH₃)₂, or 2-oxa-6-azaspiro[3.3]heptanyl.

8. The compound of claim 1, wherein R⁴ is H, CH₃, CH₂NH₂, CH₂N(CH₃)₂, C(O)N(H)CH₃, piperazinyl, C(O)OCH₃, piperazinyl-CH₃, C(O)OH, S(O₂)CH₃, C(O)NH₂, morpholinyl, CH₂OH, Cl, OCH₃, CH₂-morpholinyl, CH₂OCH₃, CH₂-piperazinyl-CH₃, CH₂CH₂N(CH₃)₂, CH₂OC(O)-phenyl, CH₂N(H)CH₃, CH₂OCH₂CH₂OCH₃, CH₂OC(O)CH₂CH₂-piperazynyl-CH₃, CH₂OC(O)CH₂CH₂N(CH₃)₂, CH₂OC(O)CH₂CH₂-morpholinyl, C(O)N(CH₃)₂, CH₂N(H)C(O)C(CH₃)NH₂, CH₂N(H)C(O)-pyrrolidinyl, CH₂N(H)Boc, CH₂N(H)C(O)CH₂N(CH₃)₂, CH₂N(H)C(O)C(NH₂)CH₂CH₂CH₂-guanidinyl, CF₃, OCH₂CH₂N(CH₃)₂, OCH₂CH₂OCH₃, azetidinyl-N(CH₃)₂, or 2-oxa-6-azaspiro[3.3]heptanyl.

9. The compound of claim 1, wherein:
R⁸ is a bond, NH, O, CH₂, or N(CH₂F);
X is 1; and
M is 1.

10. The compound of claim 1, wherein R³ is phenyl, isothiazolyl, pyridinyl, pyrazolyl, pyrimidinyl, pyrazolopyrimidinyl, triazolyl, or isoxazolyl.

11. The compound of claim 1, wherein R⁵ is H, F, CH₃, ═O, C(O)OCH₃, or CH₂OH.

12. The compound of claim 1, wherein the compound is of the formula: 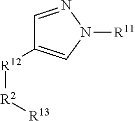
or a pharmaceutically acceptable salt thereof,
wherein
R² is NH, 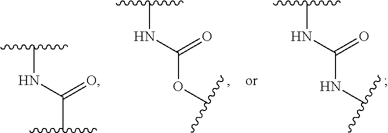 [SMILES: *NC(*)=O ; *NC(=O)O* ; *NC(=O)N*]
R¹¹ is 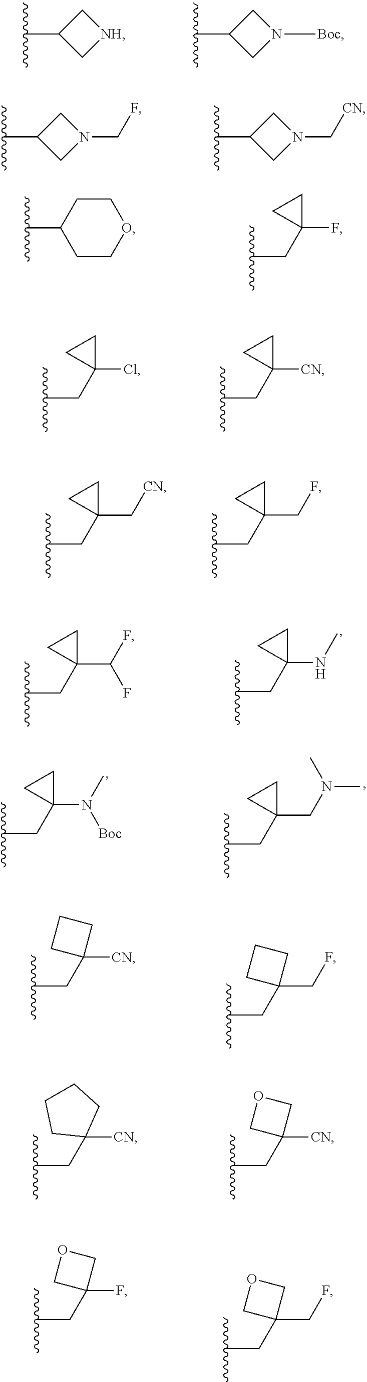 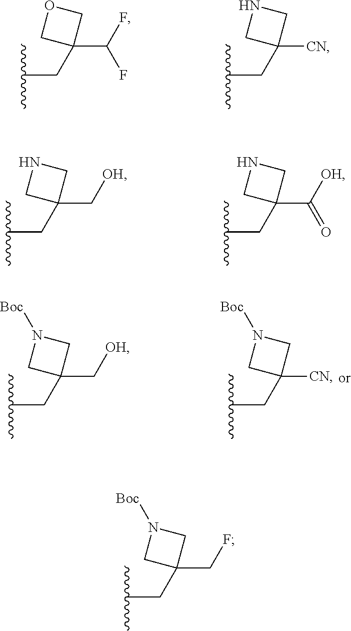
R¹² is 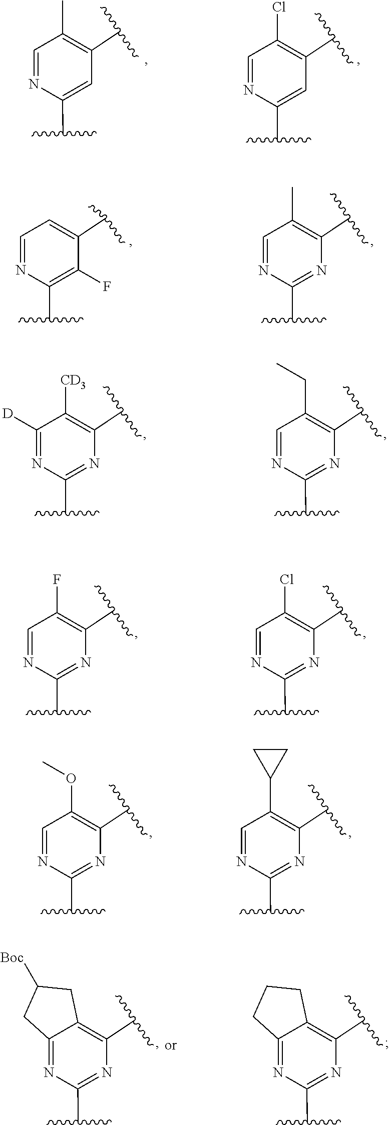
and
R¹³ is

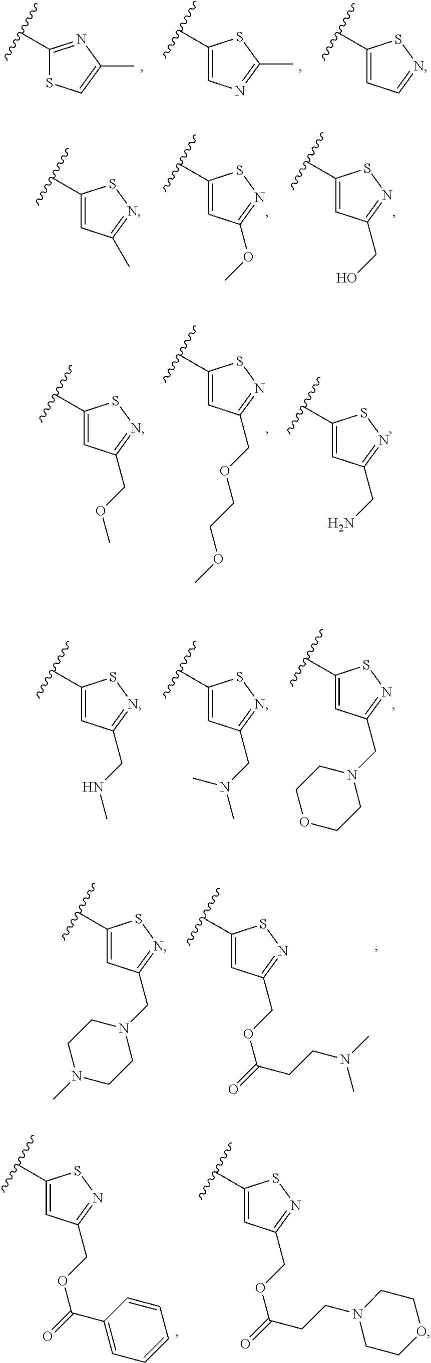

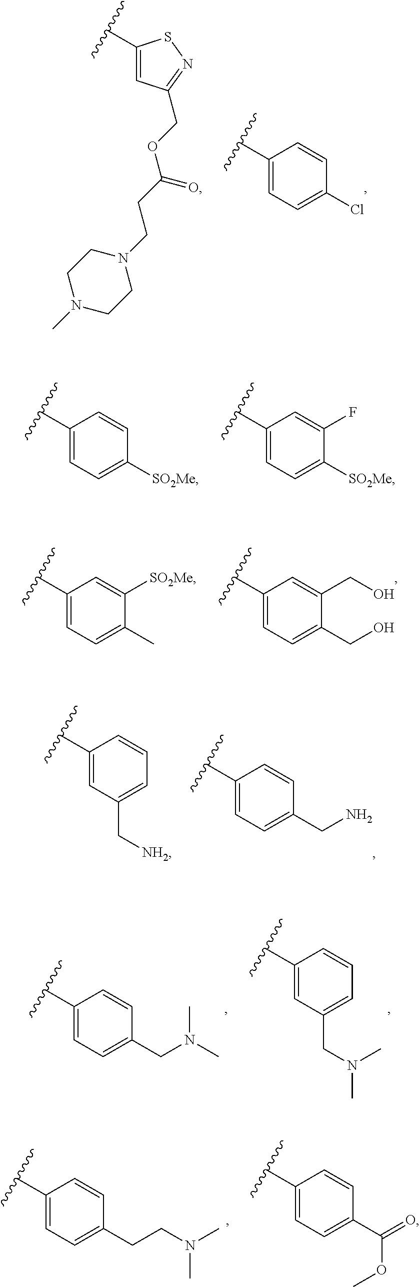

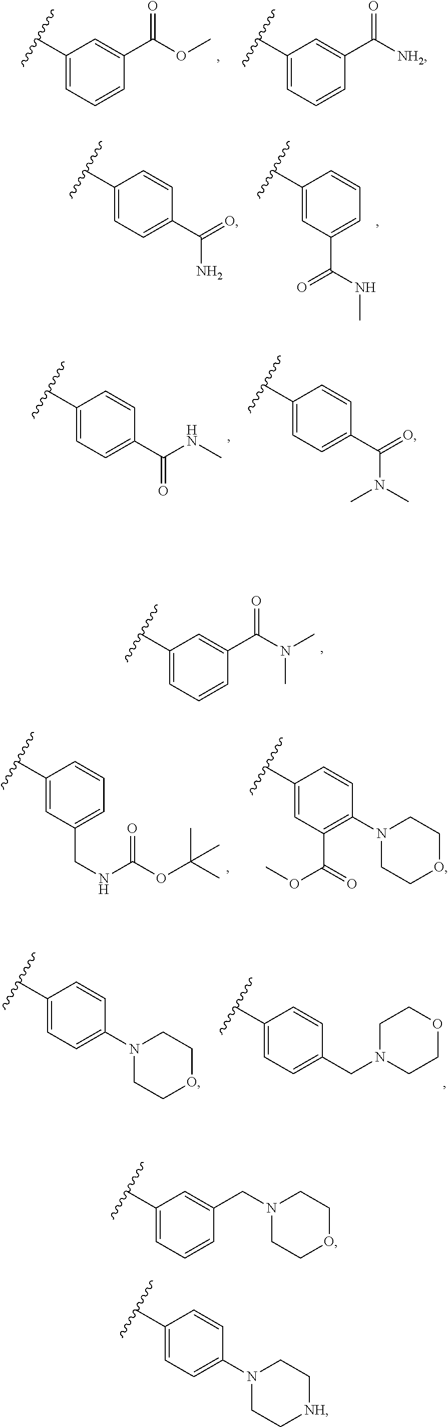

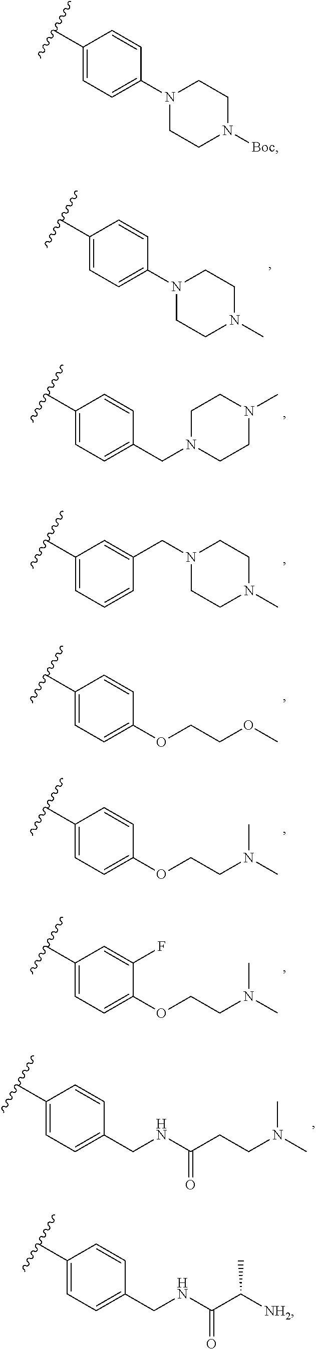

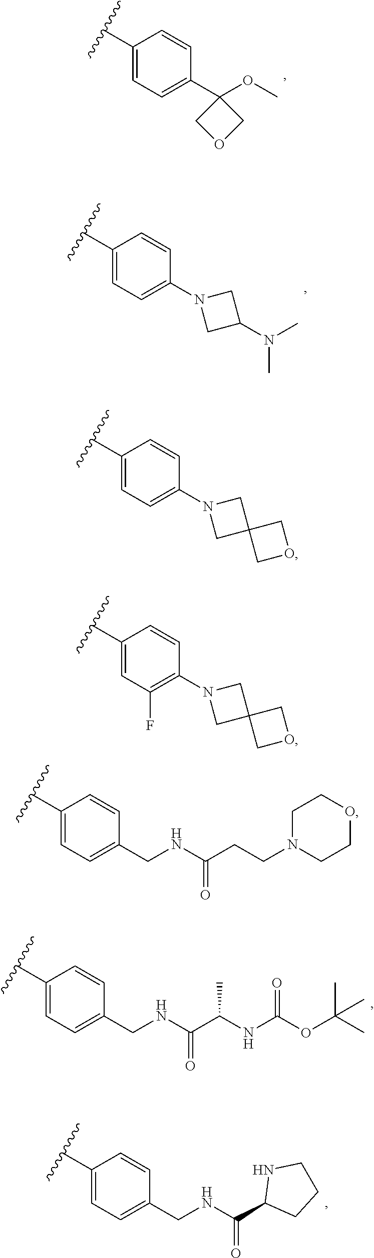

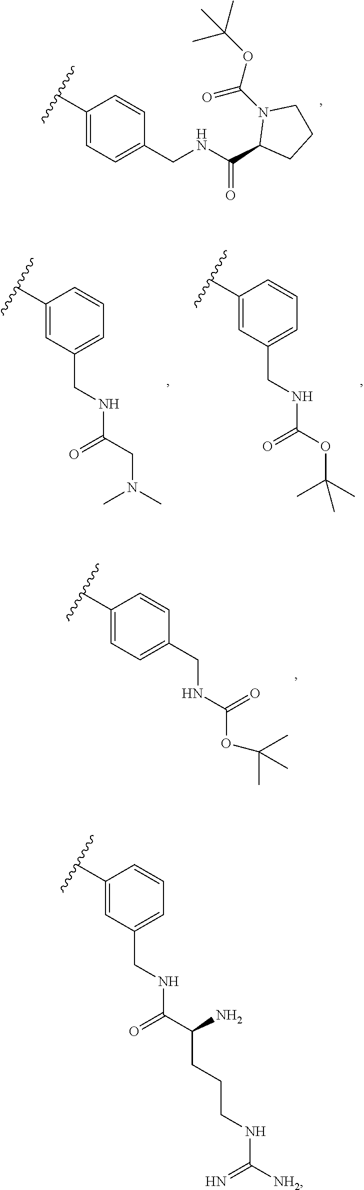 [SMILES: *c1ccc(CNC(=O)[C@@H]2CCCN2C(=O)OC(C)(C)C)cc1 ; *c1cccc(CNC(=O)CN(C)C)c1 ; *c1cccc(CNC(=O)OC(C)(C)C)c1 ; *c1ccc(CNC(=O)OC(C)(C)C)cc1 ; *c1cccc(CNC(=O)[C@@H](N)CCCNC(=N)N)c1]

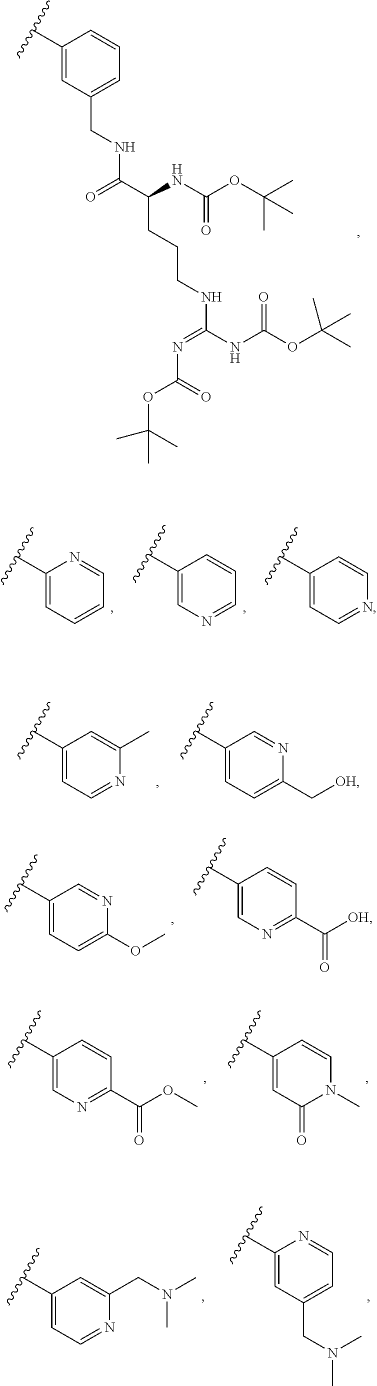

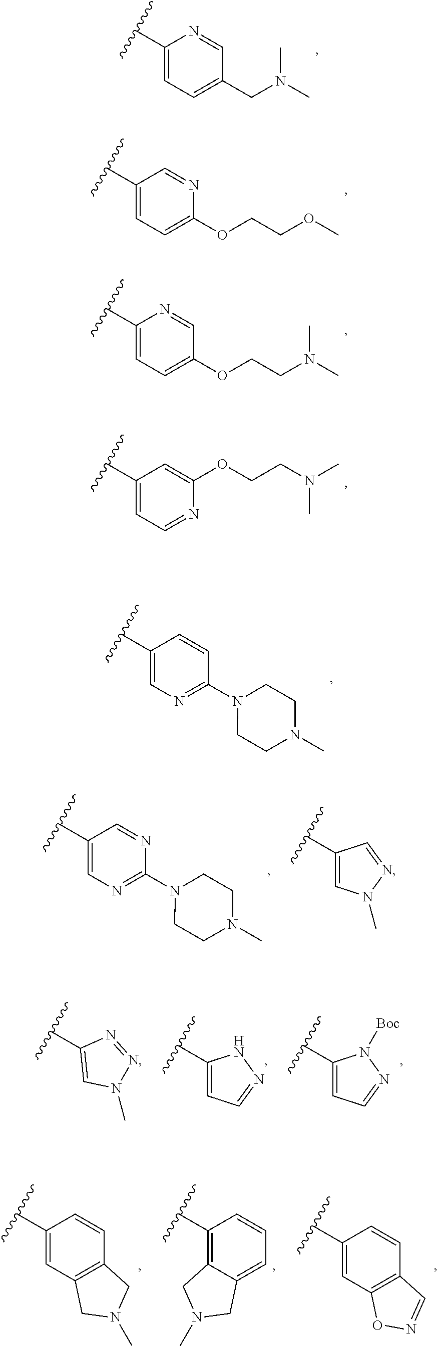

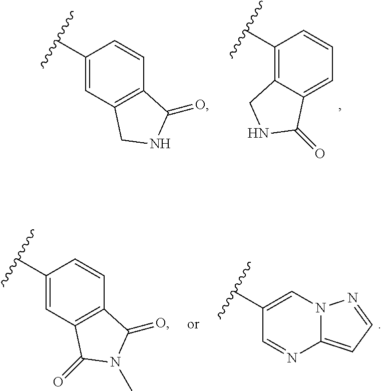 [SMILES: *c1ccc2c(c1)CNC2=O ; *c1cccc2c1CNC2=O ; *c1ccc2c(c1)C(=O)N(C)C2=O ; *c1cnc2ccnn2c1]

13. The compound of one of claims 1-12, wherein R² is NH.

14. The compound of claim 1, wherein the compound is 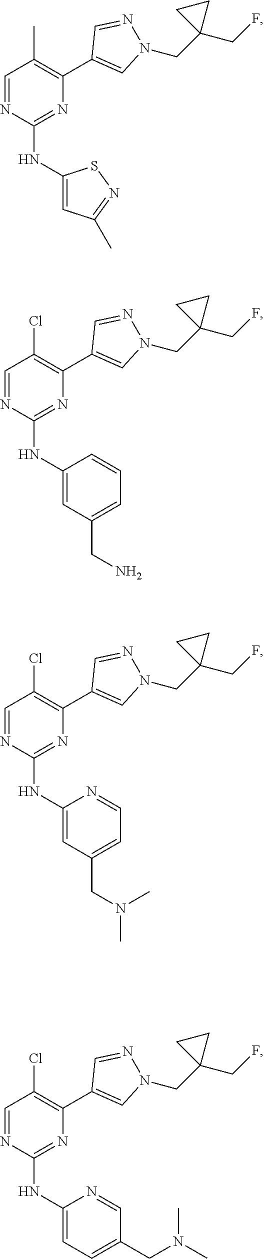 [SMILES: Cc1cc(Nc2ncc(C)c(-c3cnn(CC4(CF)CC4)c3)n2)sn1 ; NCc1cccc(Nc2ncc(Cl)c(-c3cnn(CC4(CF)CC4)c3)n2)c1 ; CN(C)Cc1ccnc(Nc2ncc(Cl)c(-c3cnn(CC4(CF)CC4)c3)n2)c1 ; CN(C)Cc1ccc(Nc2ncc(Cl)c(-c3cnn(CC4(CF)CC4)c3)n2)nc1] 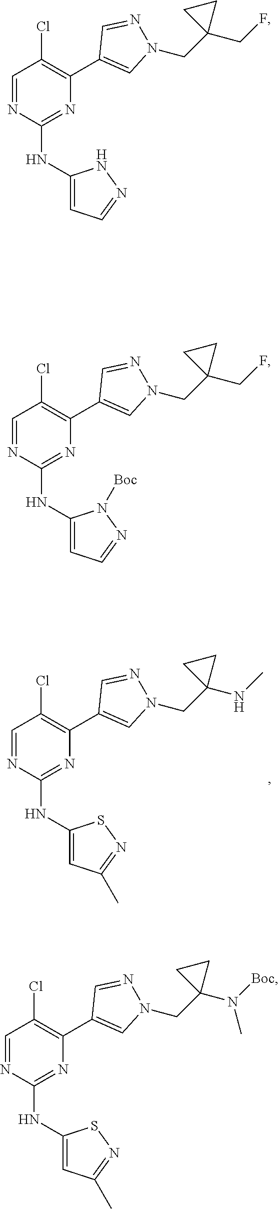 [SMILES: FCC1(Cn2cc(-c3nc(Nc4ccn[nH]4)ncc3Cl)cn2)CC1 ; CC(C)(C)OC(=O)n1nccc1Nc1ncc(Cl)c(-c2cnn(CC3(CF)CC3)c2)n1 ; CNC1(Cn2cc(-c3nc(Nc4cc(C)ns4)ncc3Cl)cn2)CC1 ; Cc1cc(Nc2ncc(Cl)c(-c3cnn(CC4(N(C)C(=O)OC(C)(C)C)CC4)c3)n2)sn1] 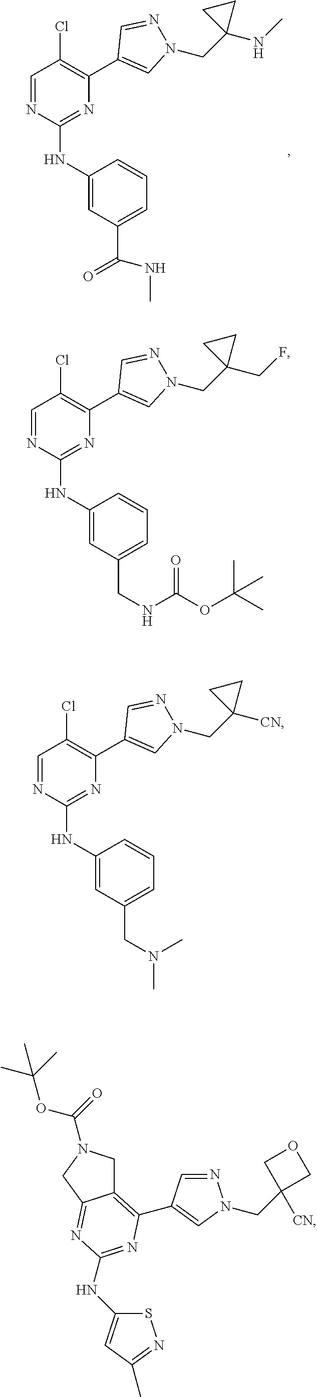 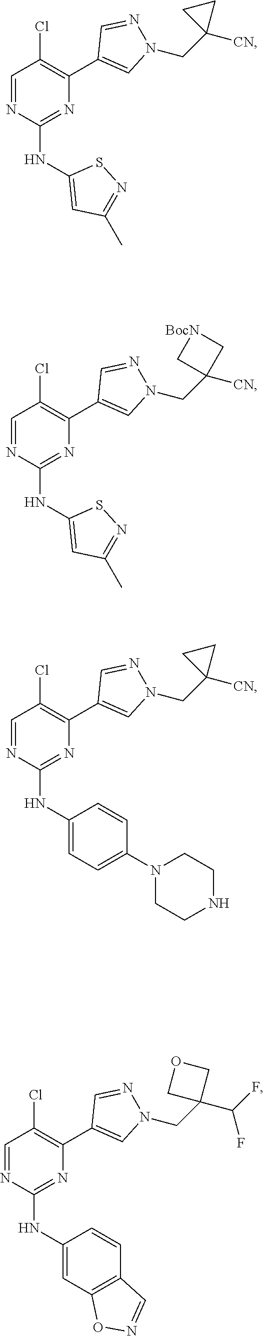 [SMILES: Cc1cc(Nc2ncc(Cl)c(-c3cnn(CC4(C#N)CC4)c3)n2)sn1 ; Cc1cc(Nc2ncc(Cl)c(-c3cnn(CC4(C#N)CN(C(=O)OC(C)(C)C)C4)c3)n2)sn1 ; N#CC1(Cn2cc(-c3nc(Nc4ccc(N5CCNCC5)cc4)ncc3Cl)cn2)CC1 ; FC(F)C1(Cn2cc(-c3nc(Nc4ccc5cnoc5c4)ncc3Cl)cn2)COC1] 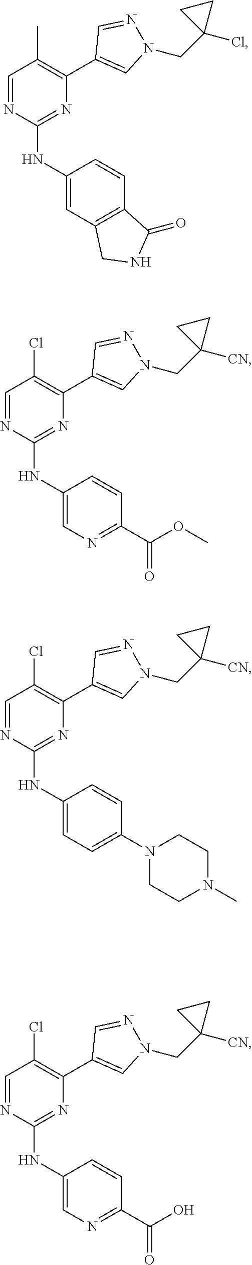 [SMILES: Cc1cnc(Nc2ccc3c(c2)CNC3=O)nc1-c1cnn(CC2(Cl)CC2)c1 ; COC(=O)c1ccc(Nc2ncc(Cl)c(-c3cnn(CC4(C#N)CC4)c3)n2)cn1 ; CN1CCN(c2ccc(Nc3ncc(Cl)c(-c4cnn(CC5(C#N)CC5)c4)n3)cc2)CC1 ; N#CC1(Cn2cc(-c3nc(Nc4ccc(C(=O)O)nc4)ncc3Cl)cn2)CC1] 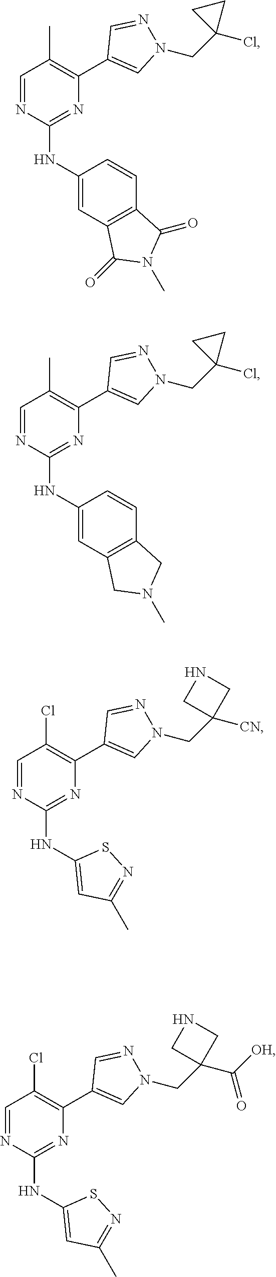 [SMILES: Cc1cnc(Nc2ccc3c(c2)C(=O)N(C)C3=O)nc1-c1cnn(CC2(Cl)CC2)c1 ; Cc1cnc(Nc2ccc3c(c2)CN(C)C3)nc1-c1cnn(CC2(Cl)CC2)c1 ; Cc1cc(Nc2ncc(Cl)c(-c3cnn(CC4(C#N)CNC4)c3)n2)sn1 ; Cc1cc(Nc2ncc(Cl)c(-c3cnn(CC4(C(=O)O)CNC4)c3)n2)sn1] 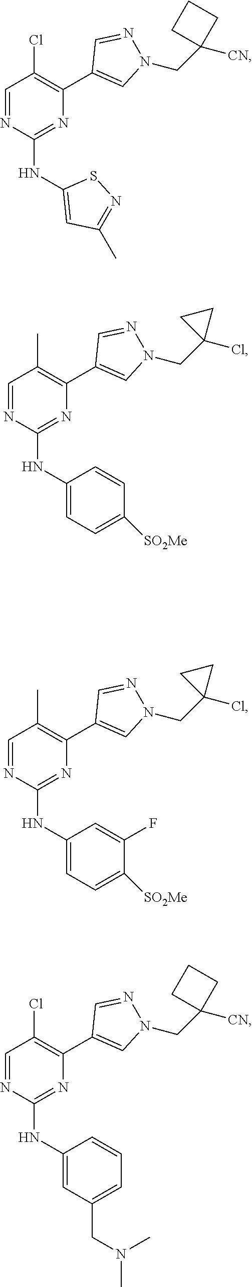 [SMILES: Cc1cc(Nc2ncc(Cl)c(-c3cnn(CC4(C#N)CCC4)c3)n2)sn1 ; Cc1cnc(Nc2ccc(S(C)(=O)=O)cc2)nc1-c1cnn(CC2(Cl)CC2)c1 ; Cc1cnc(Nc2ccc(S(C)(=O)=O)c(F)c2)nc1-c1cnn(CC2(Cl)CC2)c1 ; CN(C)Cc1cccc(Nc2ncc(Cl)c(-c3cnn(CC4(C#N)CCC4)c3)n2)c1] 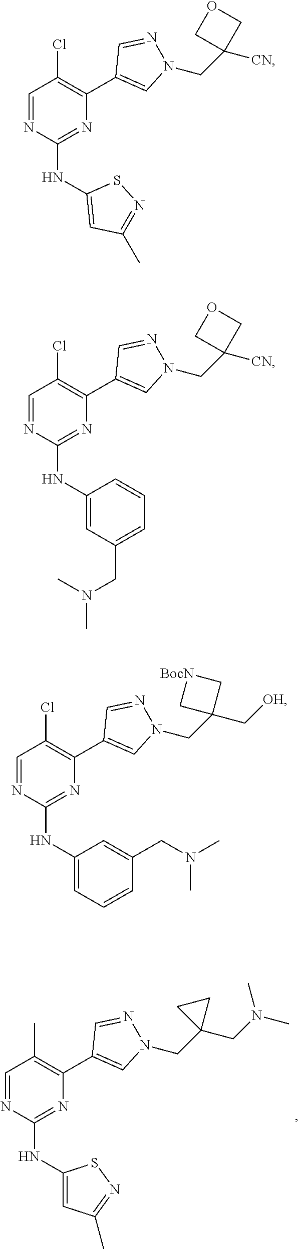 [SMILES: Cc1cc(Nc2ncc(Cl)c(-c3cnn(CC4(C#N)COC4)c3)n2)sn1 ; CN(C)Cc1cccc(Nc2ncc(Cl)c(-c3cnn(CC4(C#N)COC4)c3)n2)c1 ; CN(C)Cc1cccc(Nc2ncc(Cl)c(-c3cnn(CC4(CO)CN(C(=O)OC(C)(C)C)C4)c3)n2)c1 ; Cc1cc(Nc2ncc(C)c(-c3cnn(CC4(CN(C)C)CC4)c3)n2)sn1] 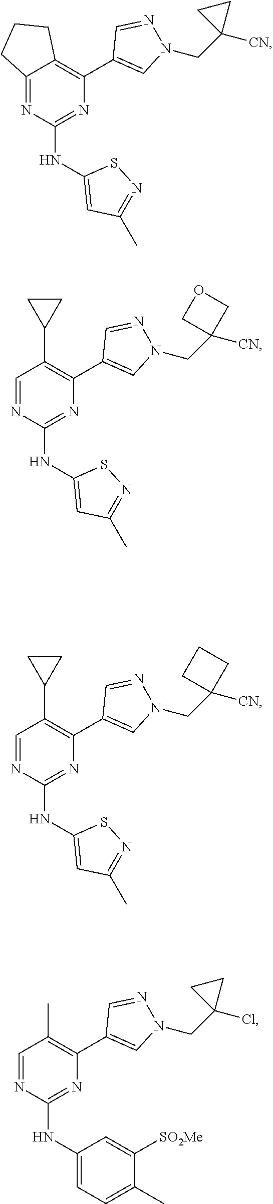 [SMILES: Cc1cc(Nc2nc3c(c(-c4cnn(CC5(C#N)CC5)c4)n2)CCC3)sn1 ; Cc1cc(Nc2ncc(C3CC3)c(-c3cnn(CC4(C#N)COC4)c3)n2)sn1 ; Cc1cc(Nc2ncc(C3CC3)c(-c3cnn(CC4(C#N)CCC4)c3)n2)sn1 ; Cc1ccc(Nc2ncc(C)c(-c3cnn(CC4(Cl)CC4)c3)n2)cc1S(C)(=O)=O] 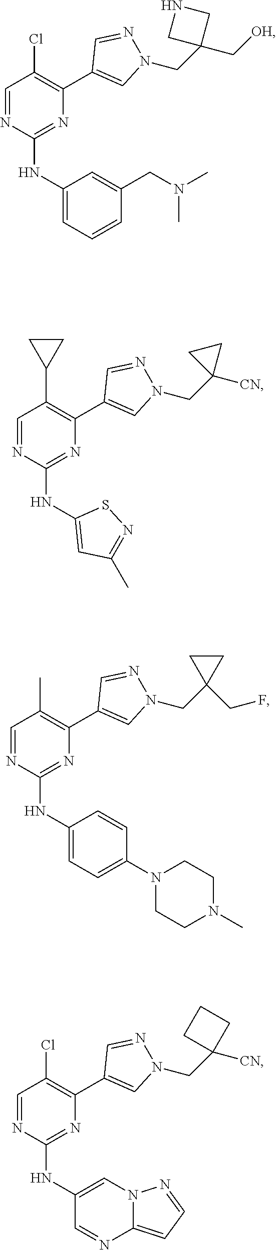 [SMILES: CN(C)Cc1cccc(Nc2ncc(Cl)c(-c3cnn(CC4(CO)CNC4)c3)n2)c1 ; Cc1cc(Nc2ncc(C3CC3)c(-c3cnn(CC4(C#N)CC4)c3)n2)sn1 ; Cc1cnc(Nc2ccc(N3CCN(C)CC3)cc2)nc1-c1cnn(CC2(CF)CC2)c1 ; N#CC1(Cn2cc(-c3nc(Nc4cnc5ccnn5c4)ncc3Cl)cn2)CCC1] 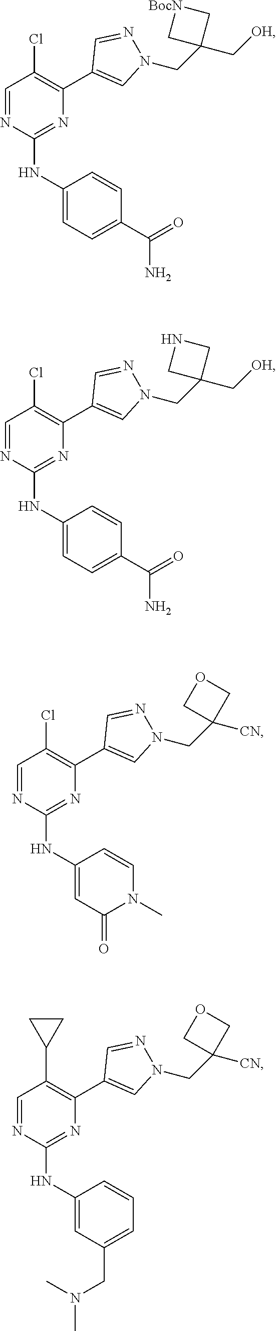 [SMILES: CC(C)(C)OC(=O)N1CC(CO)(Cn2cc(-c3nc(Nc4ccc(C(N)=O)cc4)ncc3Cl)cn2)C1 ; NC(=O)c1ccc(Nc2ncc(Cl)c(-c3cnn(CC4(CO)CNC4)c3)n2)cc1 ; Cn1ccc(Nc2ncc(Cl)c(-c3cnn(CC4(C#N)COC4)c3)n2)cc1=O ; CN(C)Cc1cccc(Nc2ncc(C3CC3)c(-c3cnn(CC4(C#N)COC4)c3)n2)c1] 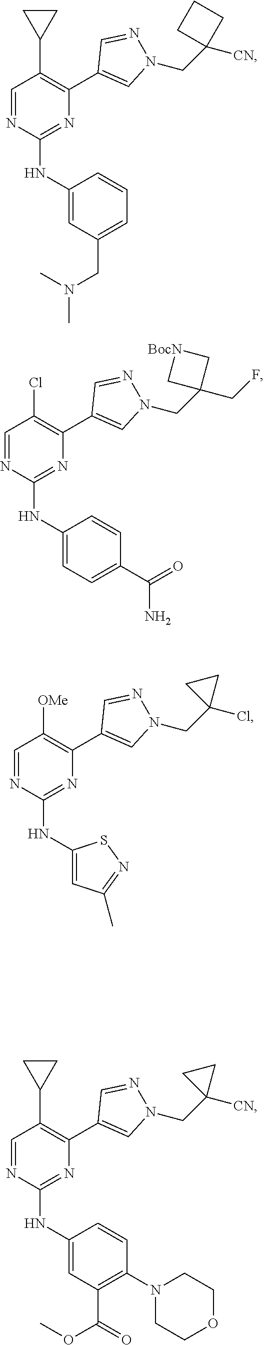 [SMILES: CN(C)Cc1cccc(Nc2ncc(C3CC3)c(-c3cnn(CC4(C#N)CCC4)c3)n2)c1 ; CC(C)(C)OC(=O)N1CC(CF)(Cn2cc(-c3nc(Nc4ccc(C(N)=O)cc4)ncc3Cl)cn2)C1 ; COc1cnc(Nc2cc(C)ns2)nc1-c1cnn(CC2(Cl)CC2)c1 ; COC(=O)c1cc(Nc2ncc(C3CC3)c(-c3cnn(CC4(C#N)CC4)c3)n2)ccc1N1CCOCC1] 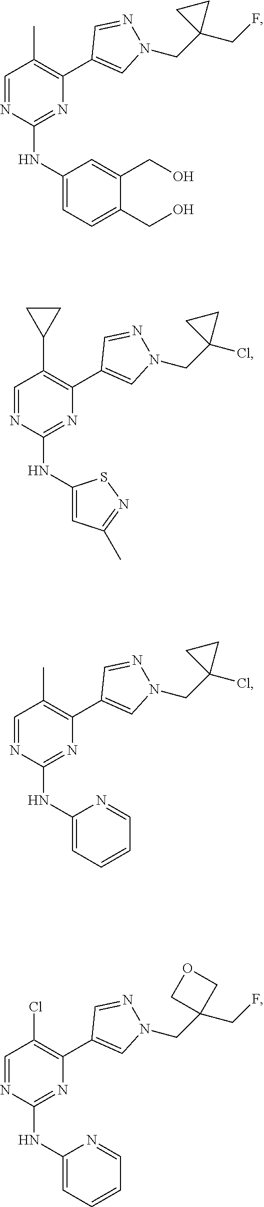 [SMILES: Cc1cnc(Nc2ccc(CO)c(CO)c2)nc1-c1cnn(CC2(CF)CC2)c1 ; Cc1cc(Nc2ncc(C3CC3)c(-c3cnn(CC4(Cl)CC4)c3)n2)sn1 ; Cc1cnc(Nc2ccccn2)nc1-c1cnn(CC2(Cl)CC2)c1 ; FCC1(Cn2cc(-c3nc(Nc4ccccn4)ncc3Cl)cn2)COC1] 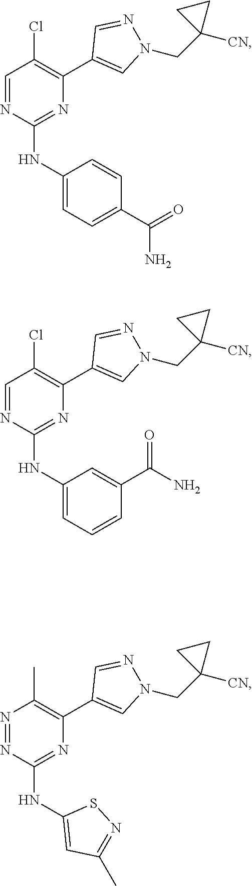 [SMILES: N#CC1(Cn2cc(-c3nc(Nc4ccc(C(N)=O)cc4)ncc3Cl)cn2)CC1 ; N#CC1(Cn2cc(-c3nc(Nc4cccc(C(N)=O)c4)ncc3Cl)cn2)CC1 ; Cc1cc(Nc2nnc(C)c(-c3cnn(CC4(C#N)CC4)c3)n2)sn1] 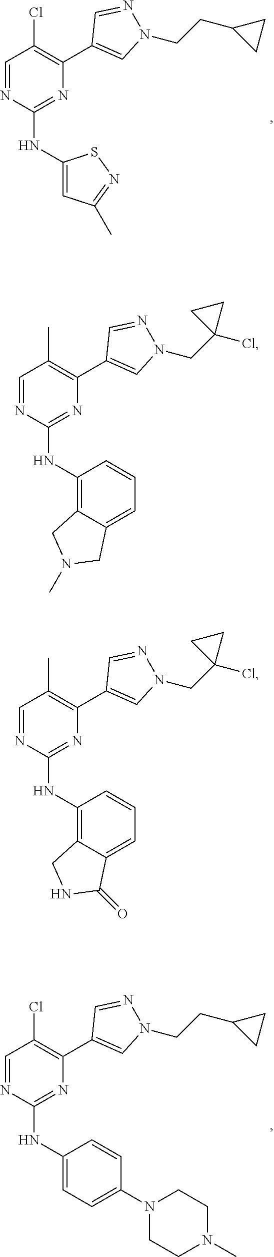 [SMILES: Cc1cc(Nc2ncc(Cl)c(-c3cnn(CCC4CC4)c3)n2)sn1 ; Cc1cnc(Nc2cccc3c2CN(C)C3)nc1-c1cnn(CC2(Cl)CC2)c1 ; Cc1cnc(Nc2cccc3c2CNC3=O)nc1-c1cnn(CC2(Cl)CC2)c1 ; CN1CCN(c2ccc(Nc3ncc(Cl)c(-c4cnn(CCC5CC5)c4)n3)cc2)CC1] 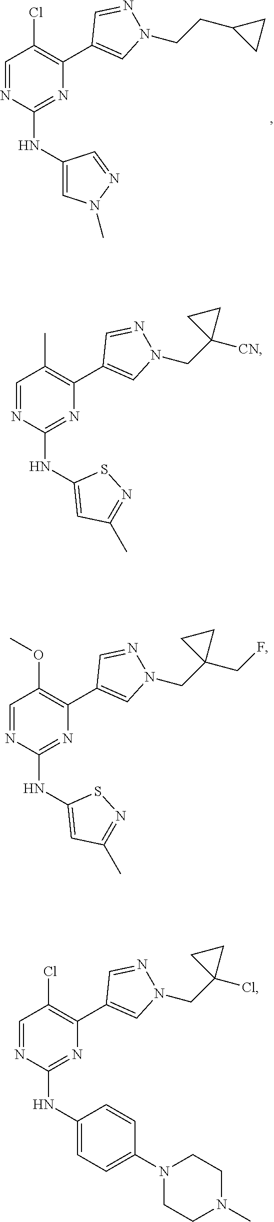 [SMILES: Cn1cc(Nc2ncc(Cl)c(-c3cnn(CCC4CC4)c3)n2)cn1 ; Cc1cc(Nc2ncc(C)c(-c3cnn(CC4(C#N)CC4)c3)n2)sn1 ; COc1cnc(Nc2cc(C)ns2)nc1-c1cnn(CC2(CF)CC2)c1 ; CN1CCN(c2ccc(Nc3ncc(Cl)c(-c4cnn(CC5(Cl)CC5)c4)n3)cc2)CC1] 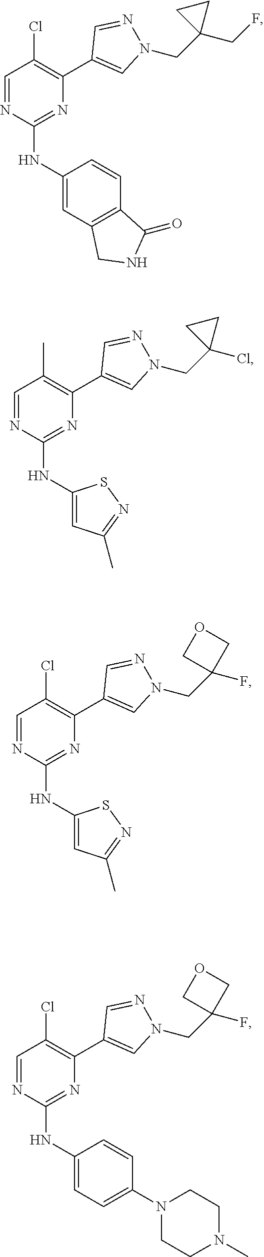 [SMILES: O=C1NCc2cc(Nc3ncc(Cl)c(-c4cnn(CC5(CF)CC5)c4)n3)ccc21 ; Cc1cc(Nc2ncc(C)c(-c3cnn(CC4(Cl)CC4)c3)n2)sn1 ; Cc1cc(Nc2ncc(Cl)c(-c3cnn(CC4(F)COC4)c3)n2)sn1 ; CN1CCN(c2ccc(Nc3ncc(Cl)c(-c4cnn(CC5(F)COC5)c4)n3)cc2)CC1] 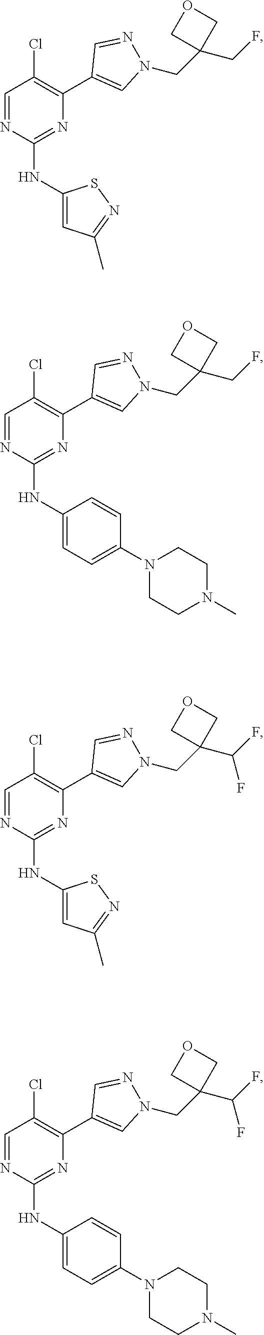 [SMILES: Cc1cc(Nc2ncc(Cl)c(-c3cnn(CC4(CF)COC4)c3)n2)sn1 ; CN1CCN(c2ccc(Nc3ncc(Cl)c(-c4cnn(CC5(CF)COC5)c4)n3)cc2)CC1 ; Cc1cc(Nc2ncc(Cl)c(-c3cnn(CC4(C(F)F)COC4)c3)n2)sn1 ; CN1CCN(c2ccc(Nc3ncc(Cl)c(-c4cnn(CC5(C(F)F)COC5)c4)n3)cc2)CC1] 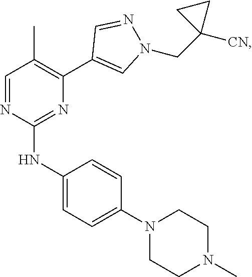 [SMILES: Cc1cnc(Nc2ccc(N3CCN(C)CC3)cc2)nc1-c1cnn(CC2(C#N)CC2)c1] 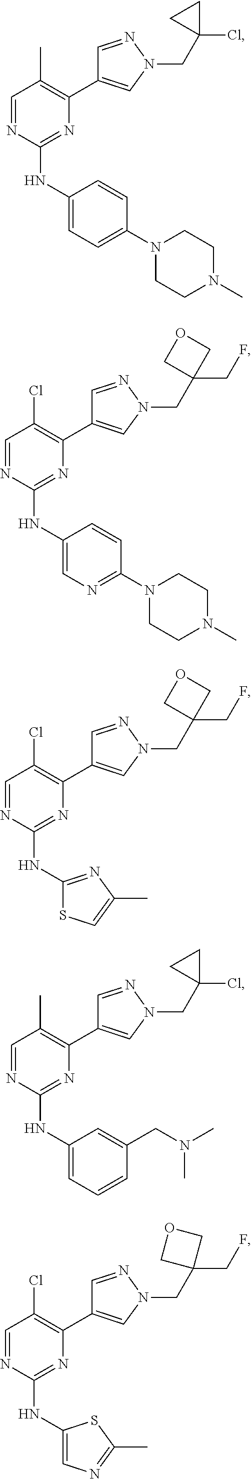 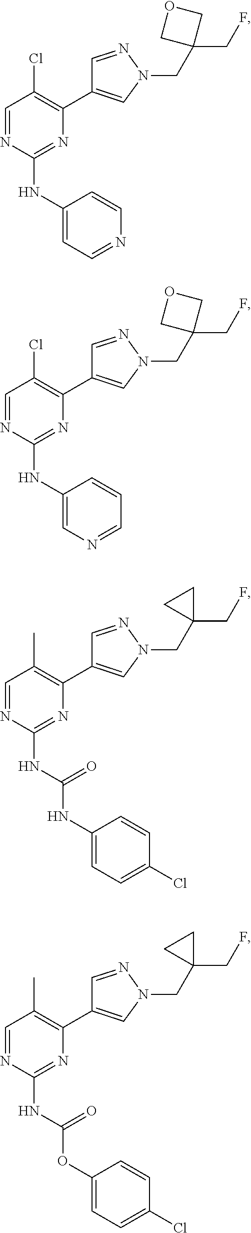 [SMILES: FCC1(Cn2cc(-c3nc(Nc4ccncc4)ncc3Cl)cn2)COC1 ; FCC1(Cn2cc(-c3nc(Nc4cccnc4)ncc3Cl)cn2)COC1 ; Cc1cnc(NC(=O)Nc2ccc(Cl)cc2)nc1-c1cnn(CC2(CF)CC2)c1 ; Cc1cnc(NC(=O)Oc2ccc(Cl)cc2)nc1-c1cnn(CC2(CF)CC2)c1] 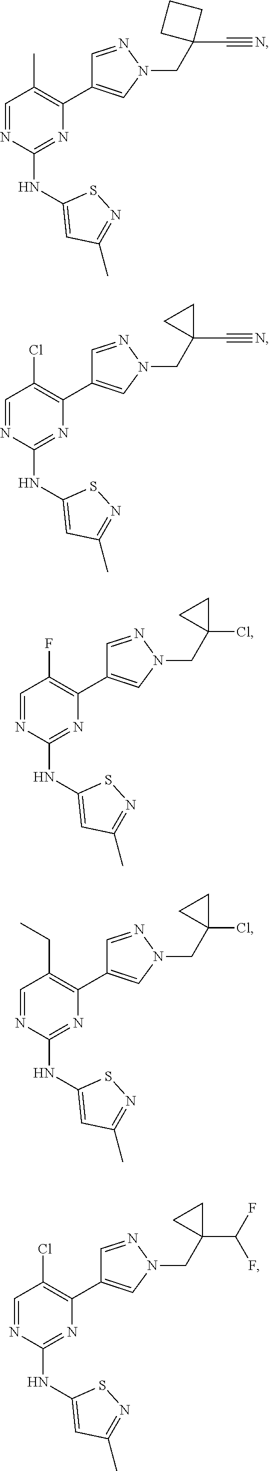 [SMILES: Cc1cc(Nc2ncc(C)c(-c3cnn(CC4(C#N)CCC4)c3)n2)sn1 ; Cc1cc(Nc2ncc(Cl)c(-c3cnn(CC4(C#N)CC4)c3)n2)sn1 ; Cc1cc(Nc2ncc(F)c(-c3cnn(CC4(Cl)CC4)c3)n2)sn1 ; CCc1cnc(Nc2cc(C)ns2)nc1-c1cnn(CC2(Cl)CC2)c1 ; Cc1cc(Nc2ncc(Cl)c(-c3cnn(CC4(C(F)F)CC4)c3)n2)sn1] 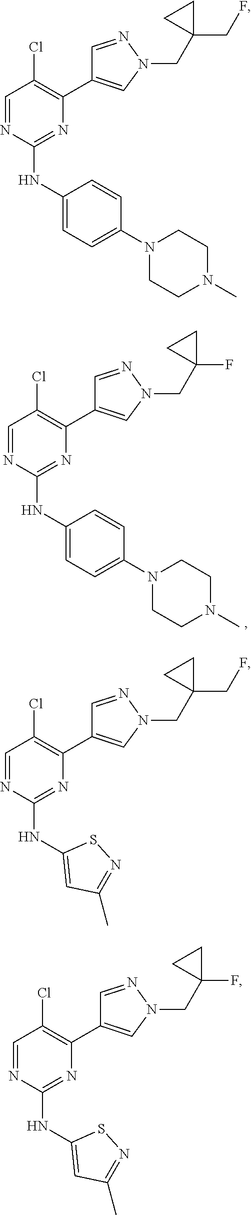 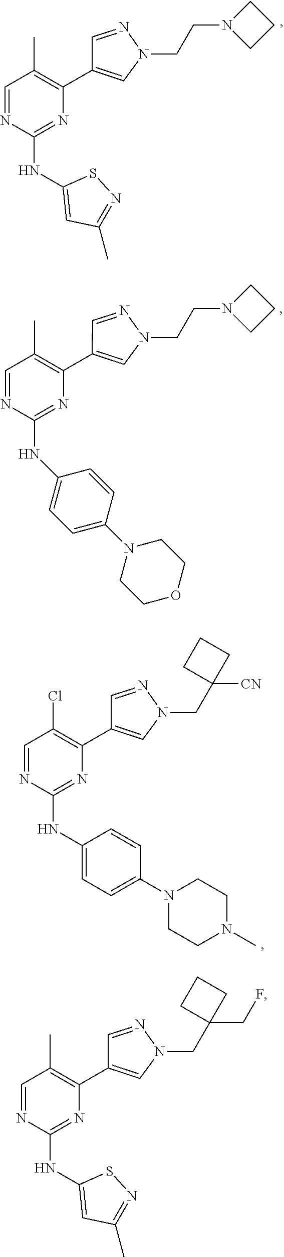 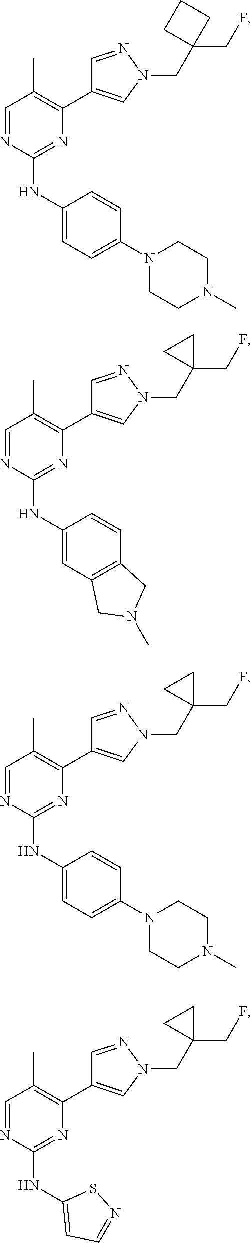 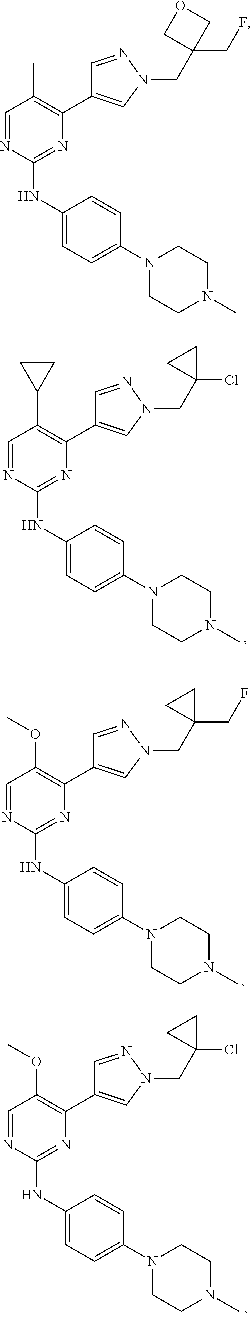 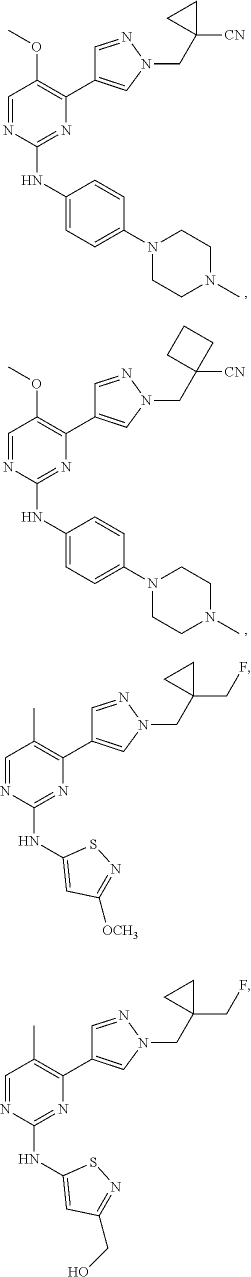 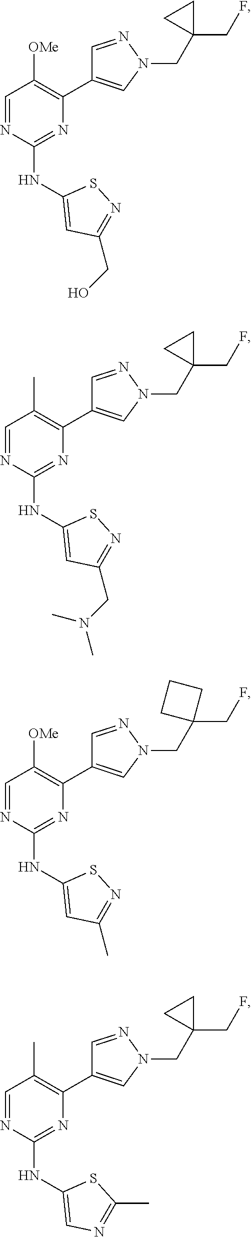 [SMILES: COc1cnc(Nc2cc(CO)ns2)nc1-c1cnn(CC2(CF)CC2)c1 ; Cc1cnc(Nc2cc(CN(C)C)ns2)nc1-c1cnn(CC2(CF)CC2)c1 ; COc1cnc(Nc2cc(C)ns2)nc1-c1cnn(CC2(CF)CCC2)c1 ; Cc1ncc(Nc2ncc(C)c(-c3cnn(CC4(CF)CC4)c3)n2)s1] 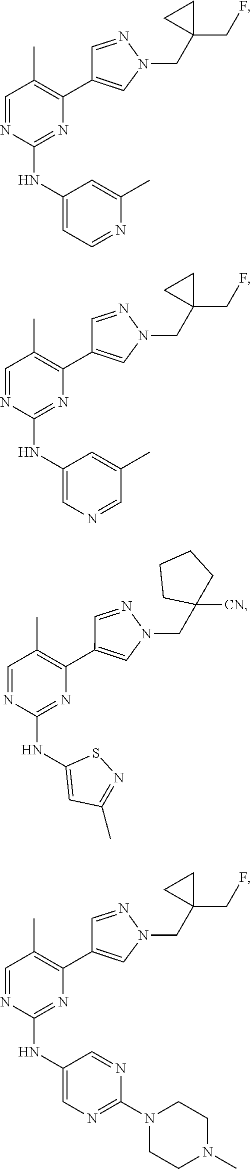 [SMILES: Cc1cc(Nc2ncc(C)c(-c3cnn(CC4(CF)CC4)c3)n2)ccn1 ; Cc1cncc(Nc2ncc(C)c(-c3cnn(CC4(CF)CC4)c3)n2)c1 ; Cc1cc(Nc2ncc(C)c(-c3cnn(CC4(C#N)CCCC4)c3)n2)sn1 ; Cc1cnc(Nc2cnc(N3CCN(C)CC3)nc2)nc1-c1cnn(CC2(CF)CC2)c1] 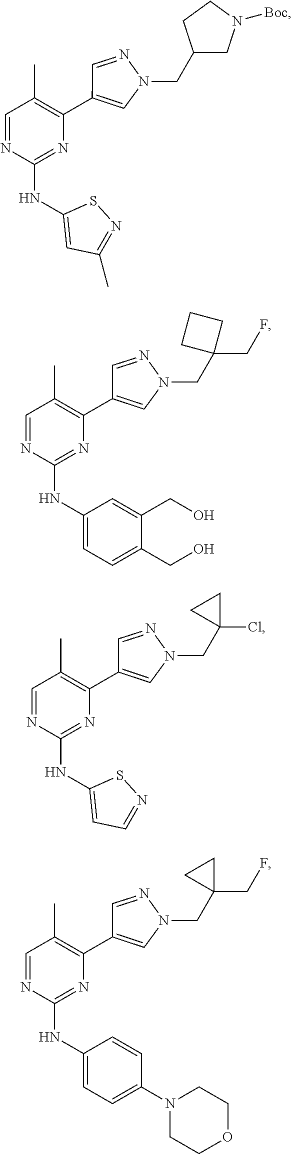 [SMILES: Cc1cc(Nc2ncc(C)c(-c3cnn(CC4CCN(C(=O)OC(C)(C)C)C4)c3)n2)sn1 ; Cc1cnc(Nc2ccc(CO)c(CO)c2)nc1-c1cnn(CC2(CF)CCC2)c1 ; Cc1cnc(Nc2ccns2)nc1-c1cnn(CC2(Cl)CC2)c1 ; Cc1cnc(Nc2ccc(N3CCOCC3)cc2)nc1-c1cnn(CC2(CF)CC2)c1] 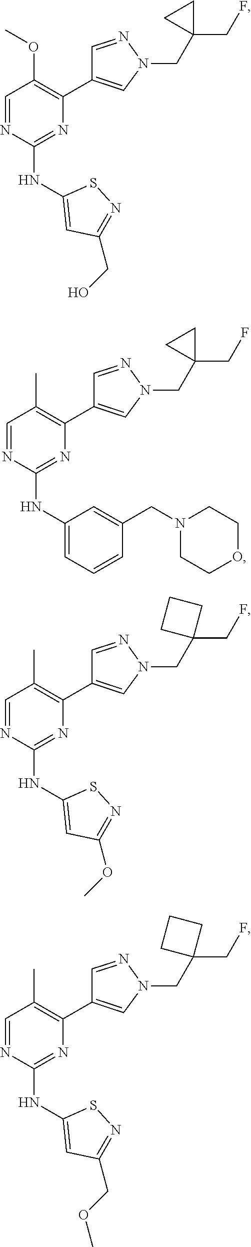 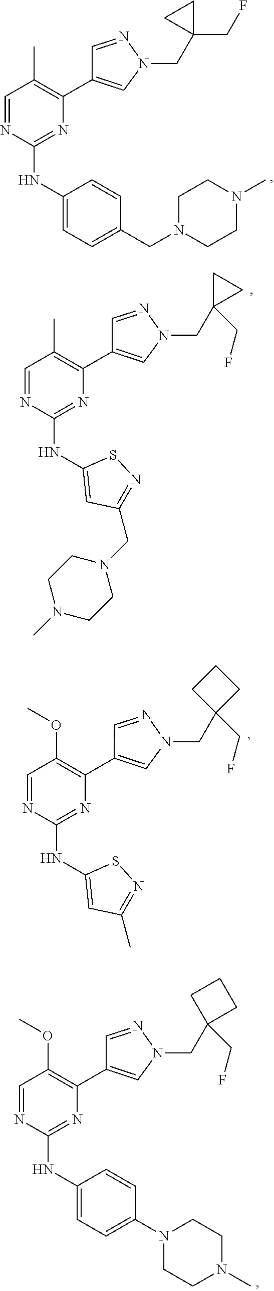 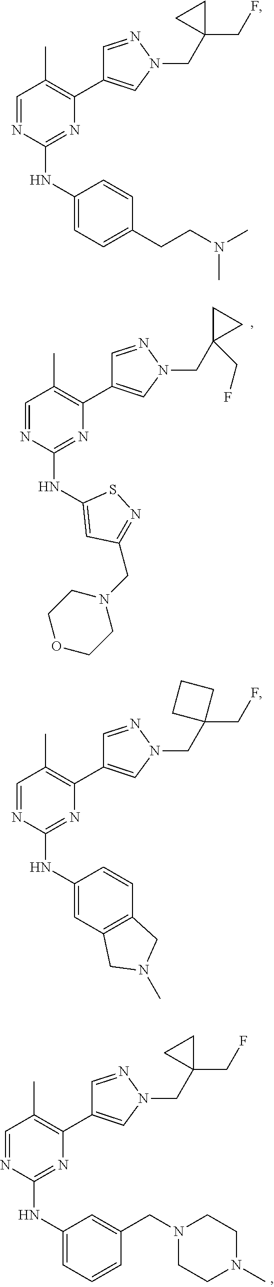 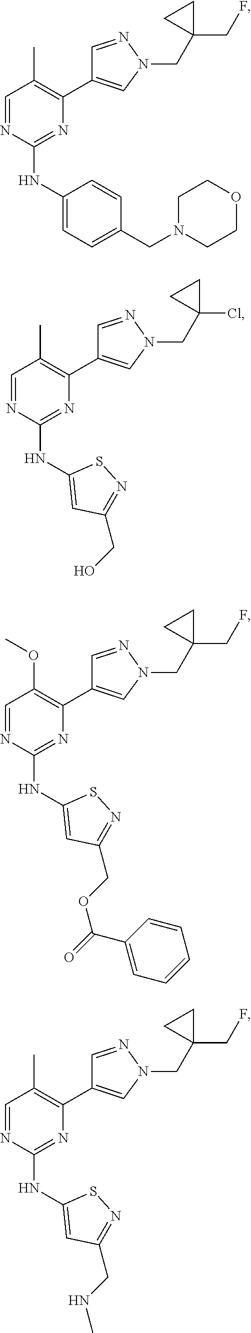 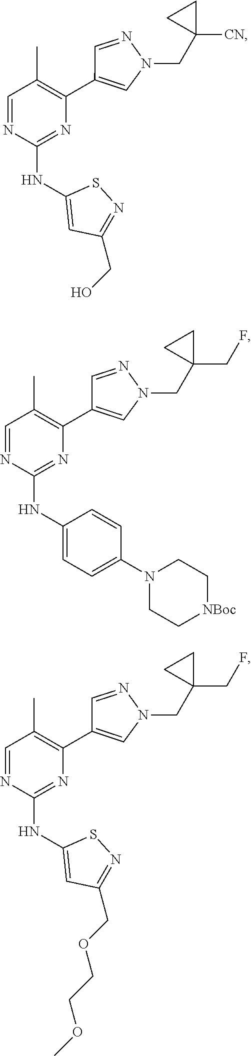 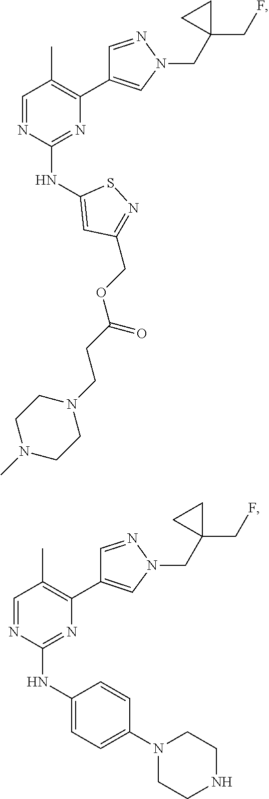 [SMILES: Cc1cnc(Nc2cc(COC(=O)CCN3CCN(C)CC3)ns2)nc1-c1cnn(CC2(CF)CC2)c1 ; Cc1cnc(Nc2ccc(N3CCNCC3)cc2)nc1-c1cnn(CC2(CF)CC2)c1] 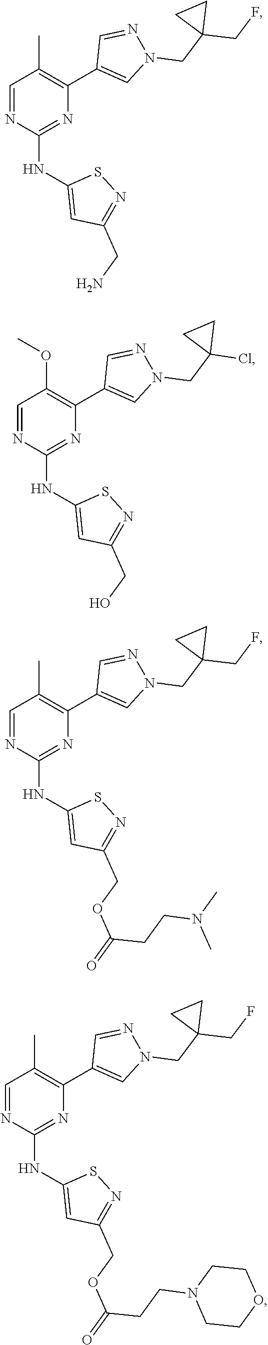 [SMILES: Cc1cnc(Nc2cc(CN)ns2)nc1-c1cnn(CC2(CF)CC2)c1 ; COc1cnc(Nc2cc(CO)ns2)nc1-c1cnn(CC2(Cl)CC2)c1 ; Cc1cnc(Nc2cc(COC(=O)CCN(C)C)ns2)nc1-c1cnn(CC2(CF)CC2)c1 ; Cc1cnc(Nc2cc(COC(=O)CCN3CCOCC3)ns2)nc1-c1cnn(CC2(CF)CC2)c1] 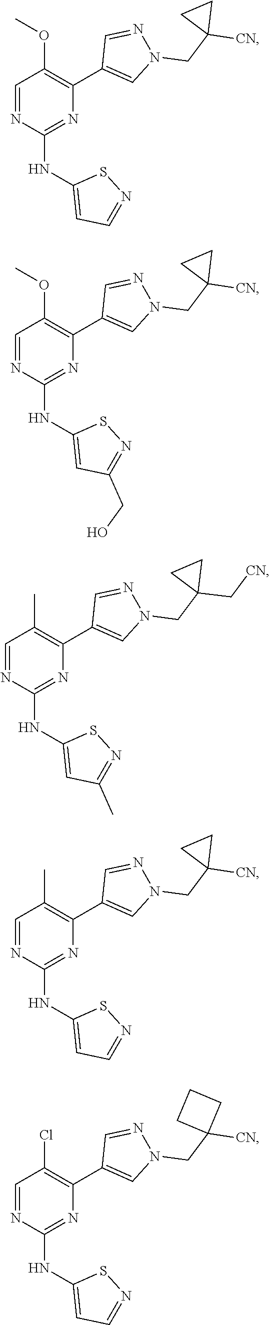 [SMILES: COc1cnc(Nc2ccns2)nc1-c1cnn(CC2(C#N)CC2)c1 ; COc1cnc(Nc2cc(CO)ns2)nc1-c1cnn(CC2(C#N)CC2)c1 ; Cc1cc(Nc2ncc(C)c(-c3cnn(CC4(CC#N)CC4)c3)n2)sn1 ; Cc1cnc(Nc2ccns2)nc1-c1cnn(CC2(C#N)CC2)c1 ; N#CC1(Cn2cc(-c3nc(Nc4ccns4)ncc3Cl)cn2)CCC1] 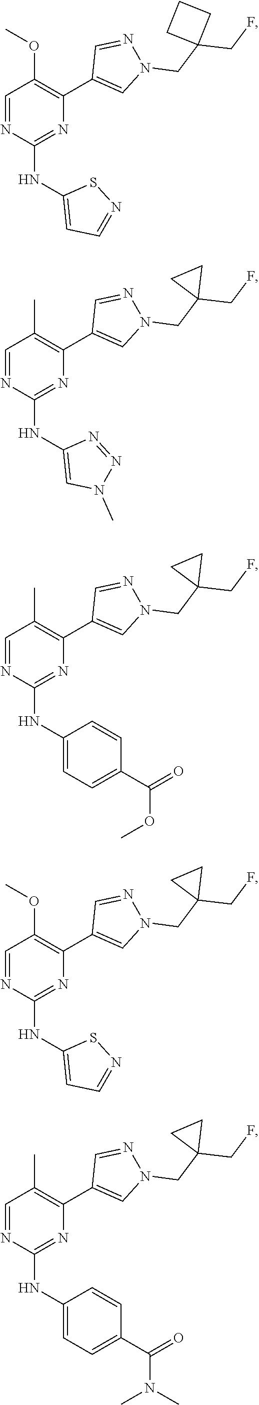 [SMILES: COc1cnc(Nc2ccns2)nc1-c1cnn(CC2(CF)CCC2)c1 ; Cc1cnc(Nc2cn(C)nn2)nc1-c1cnn(CC2(CF)CC2)c1 ; COC(=O)c1ccc(Nc2ncc(C)c(-c3cnn(CC4(CF)CC4)c3)n2)cc1 ; COc1cnc(Nc2ccns2)nc1-c1cnn(CC2(CF)CC2)c1 ; Cc1cnc(Nc2ccc(C(=O)N(C)C)cc2)nc1-c1cnn(CC2(CF)CC2)c1] 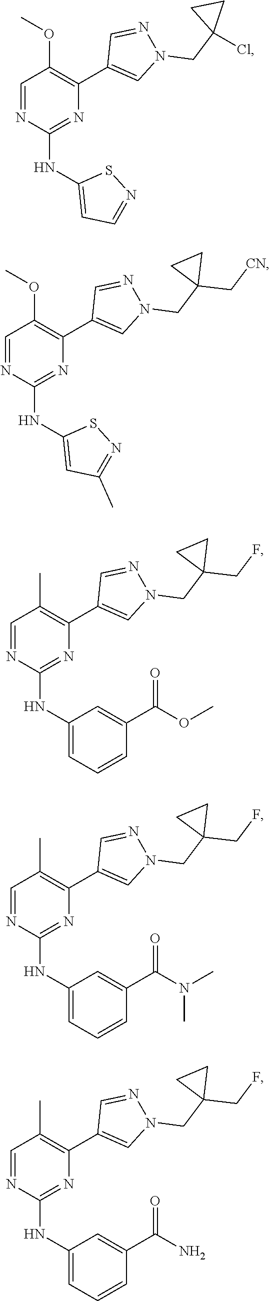 [SMILES: COc1cnc(Nc2ccns2)nc1-c1cnn(CC2(Cl)CC2)c1 ; COc1cnc(Nc2cc(C)ns2)nc1-c1cnn(CC2(CC#N)CC2)c1 ; COC(=O)c1cccc(Nc2ncc(C)c(-c3cnn(CC4(CF)CC4)c3)n2)c1 ; Cc1cnc(Nc2cccc(C(=O)N(C)C)c2)nc1-c1cnn(CC2(CF)CC2)c1 ; Cc1cnc(Nc2cccc(C(N)=O)c2)nc1-c1cnn(CC2(CF)CC2)c1] 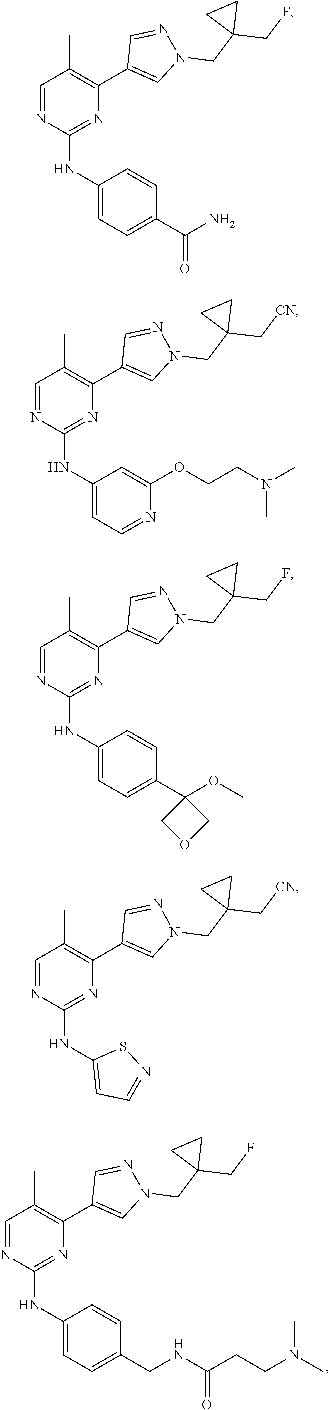 [SMILES: Cc1cnc(Nc2ccc(C(N)=O)cc2)nc1-c1cnn(CC2(CF)CC2)c1 ; Cc1cnc(Nc2ccnc(OCCN(C)C)c2)nc1-c1cnn(CC2(CC#N)CC2)c1 ; COC1(c2ccc(Nc3ncc(C)c(-c4cnn(CC5(CF)CC5)c4)n3)cc2)COC1 ; Cc1cnc(Nc2ccns2)nc1-c1cnn(CC2(CC#N)CC2)c1 ; Cc1cnc(Nc2ccc(CNC(=O)CCN(C)C)cc2)nc1-c1cnn(CC2(CF)CC2)c1] 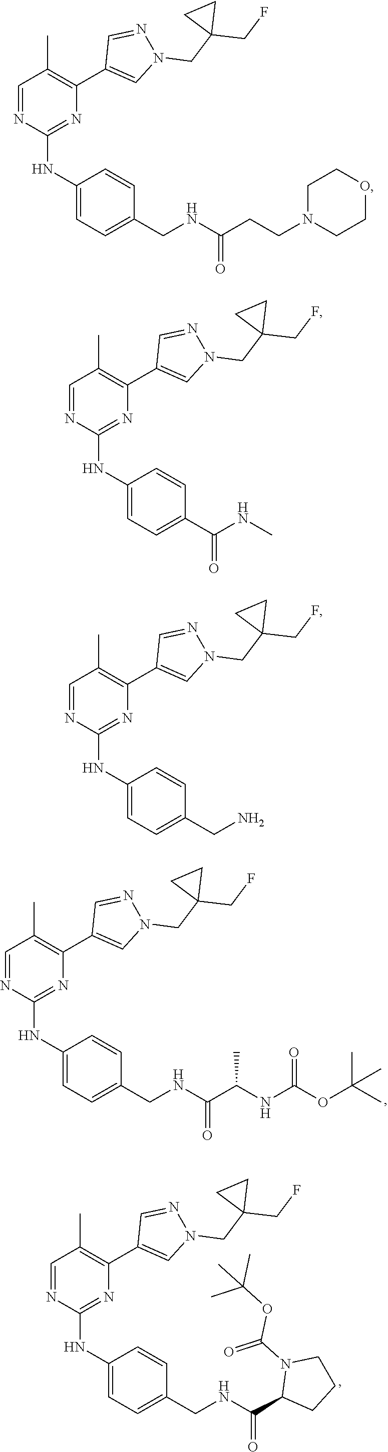 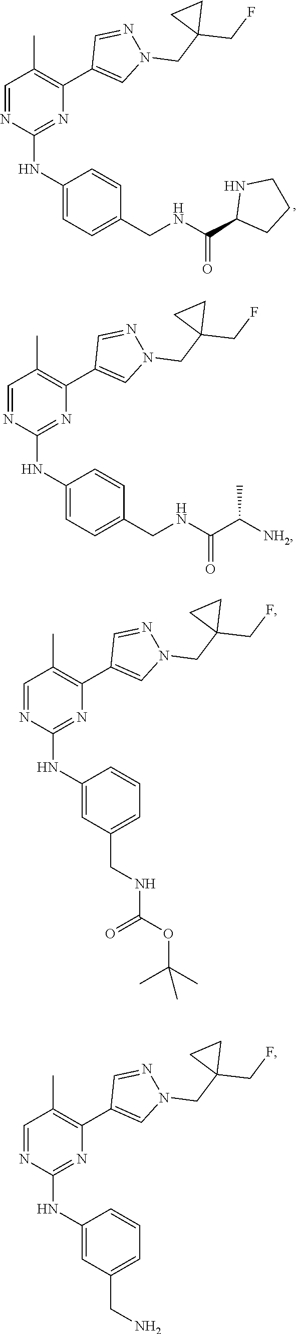 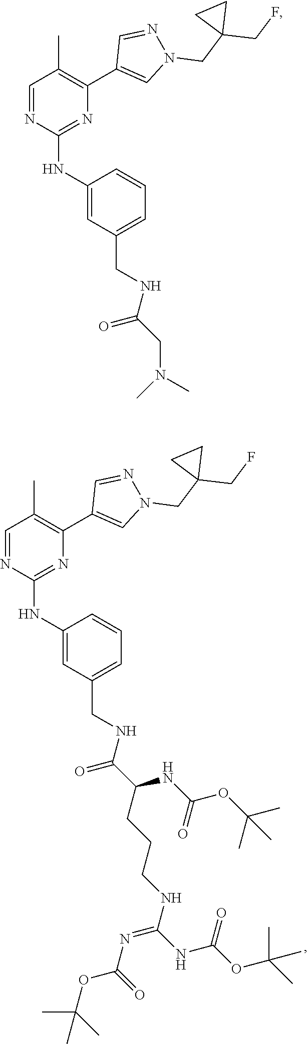 [SMILES: Cc1cnc(Nc2cccc(CNC(=O)CN(C)C)c2)nc1-c1cnn(CC2(CF)CC2)c1 ; Cc1cnc(Nc2cccc(CNC(=O)[C@H](CCCN/C(=N/C(=O)OC(C)(C)C)NC(=O)OC(C)(C)C)NC(=O)OC(C)(C)C)c2)nc1-c1cnn(CC2(CF)CC2)c1] 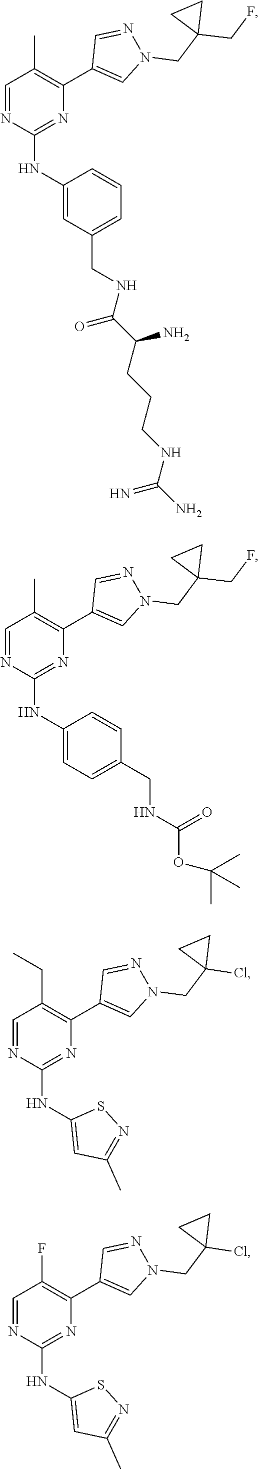 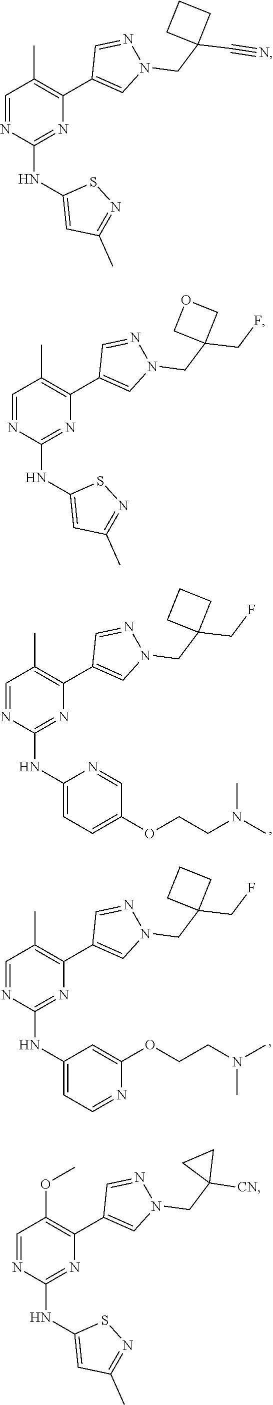 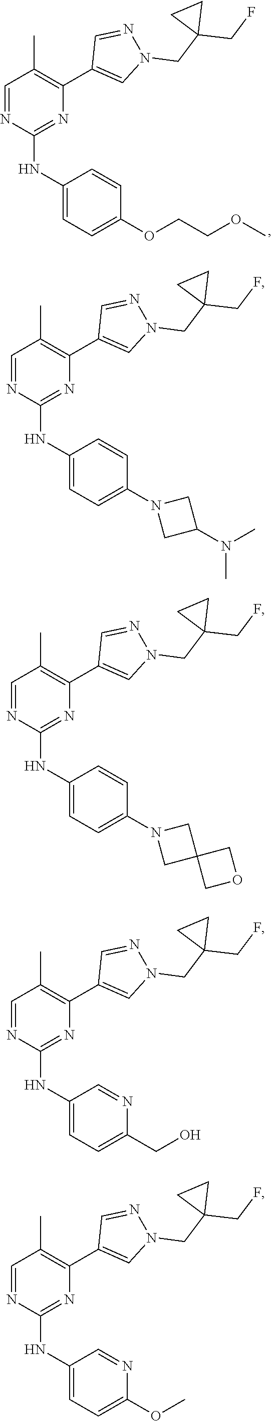 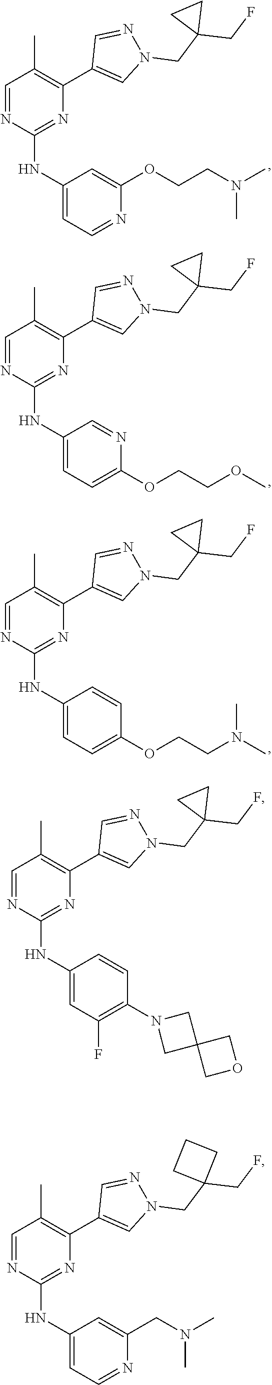 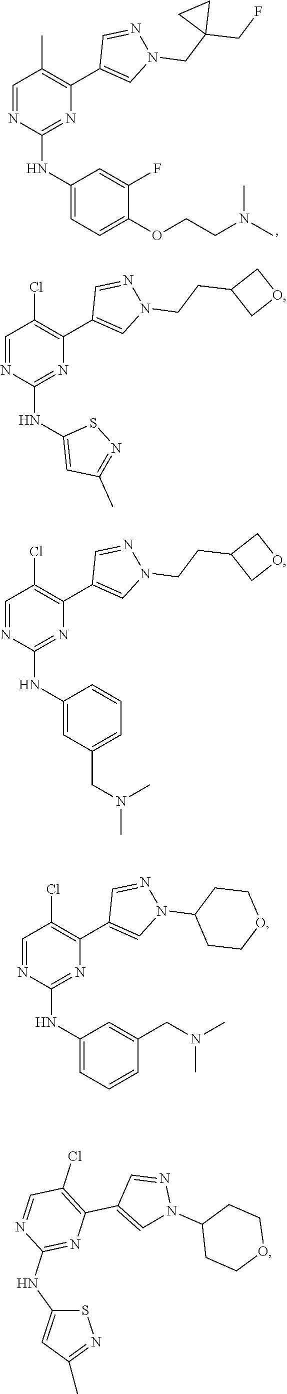 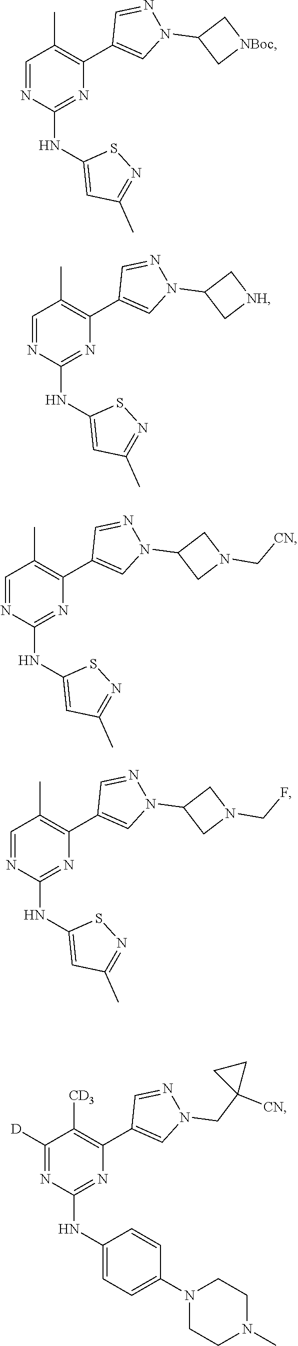 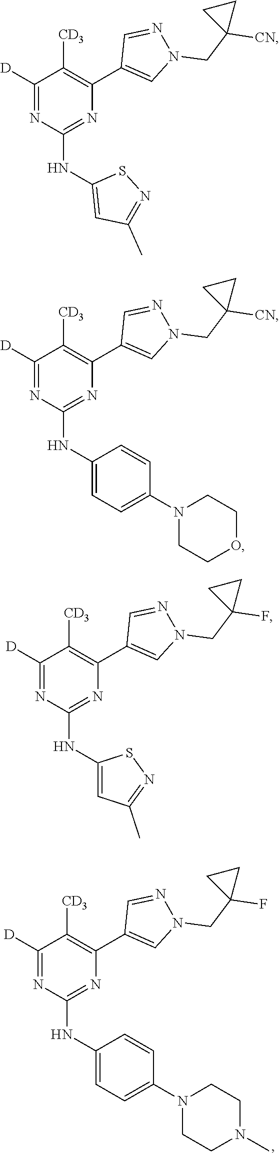 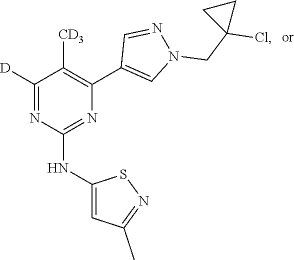 [SMILES: [2H]c1nc(Nc2cc(C)ns2)nc(-c2cnn(CC3(Cl)CC3)c2)c1C([2H])([2H])[2H]] 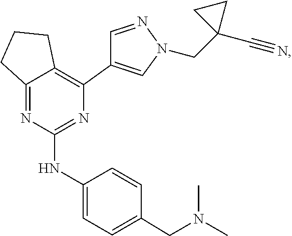 [SMILES: CN(C)Cc1ccc(Nc2nc3c(c(-c4cnn(CC5(C#N)CC5)c4)n2)CCC3)cc1]
or a pharmaceutically acceptable salt thereof.

15. A compound of the formula: 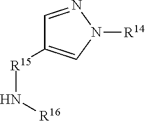
or a pharmaceutically acceptable salt thereof,
wherein
R¹⁴ is 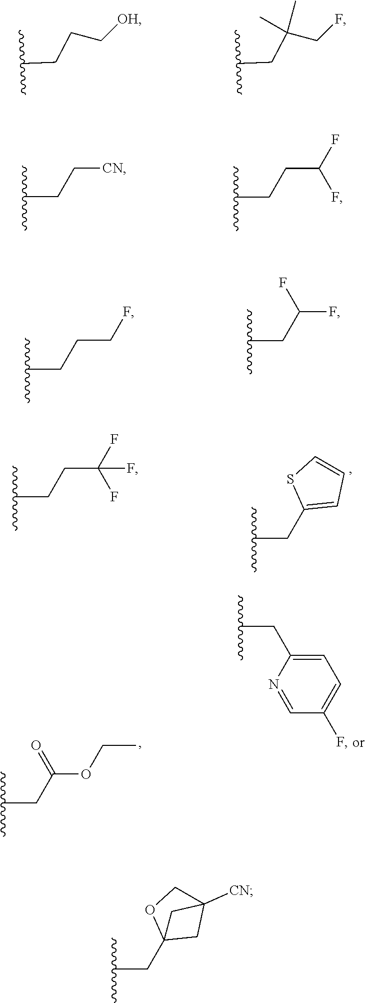
R¹⁵ is 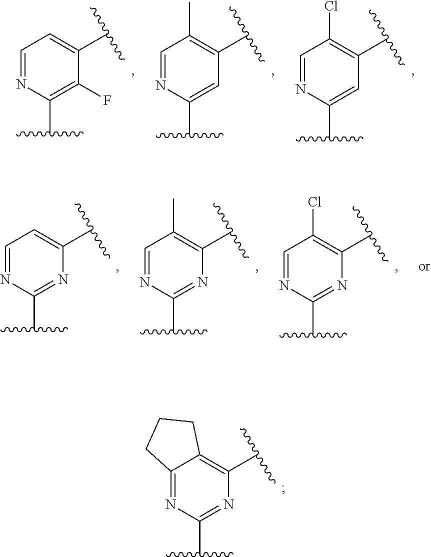
and
R¹⁶ is

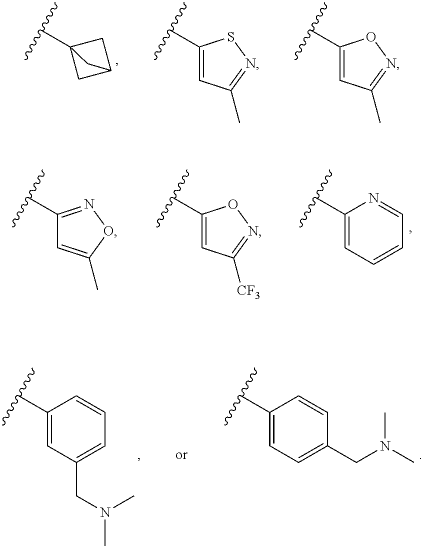

16. The compound of claim 15, wherein the compound is 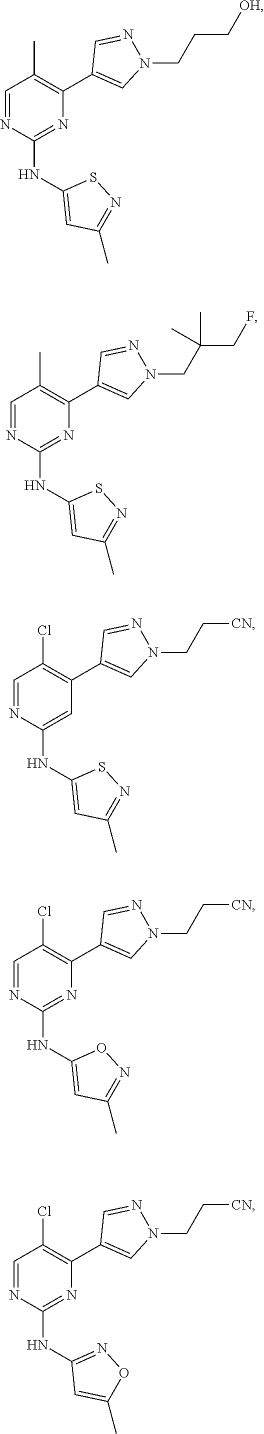 [SMILES: Cc1cc(Nc2ncc(C)c(-c3cnn(CCCO)c3)n2)sn1 ; Cc1cc(Nc2ncc(C)c(-c3cnn(CC(C)(C)CF)c3)n2)sn1 ; Cc1cc(Nc2cc(-c3cnn(CCC#N)c3)c(Cl)cn2)sn1 ; Cc1cc(Nc2ncc(Cl)c(-c3cnn(CCC#N)c3)n2)on1 ; Cc1cc(Nc2ncc(Cl)c(-c3cnn(CCC#N)c3)n2)no1] 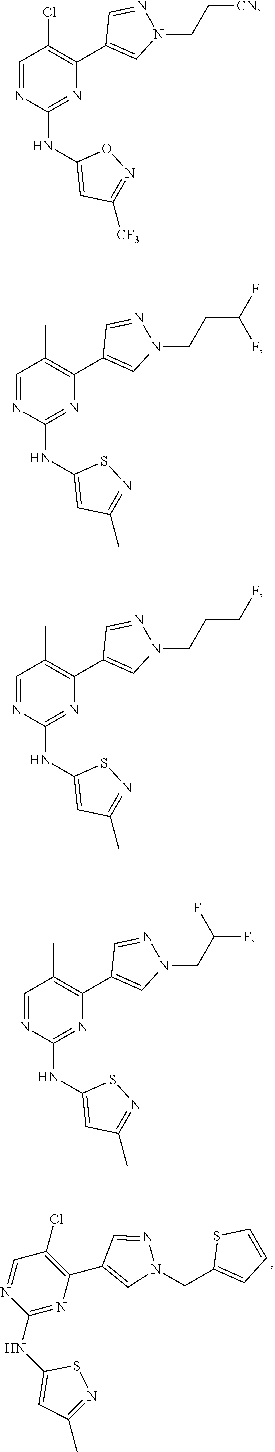 [SMILES: N#CCCn1cc(-c2nc(Nc3cc(C(F)(F)F)no3)ncc2Cl)cn1 ; Cc1cc(Nc2ncc(C)c(-c3cnn(CCC(F)F)c3)n2)sn1 ; Cc1cc(Nc2ncc(C)c(-c3cnn(CCCF)c3)n2)sn1 ; Cc1cc(Nc2ncc(C)c(-c3cnn(CC(F)F)c3)n2)sn1 ; Cc1cc(Nc2ncc(Cl)c(-c3cnn(Cc4cccs4)c3)n2)sn1] 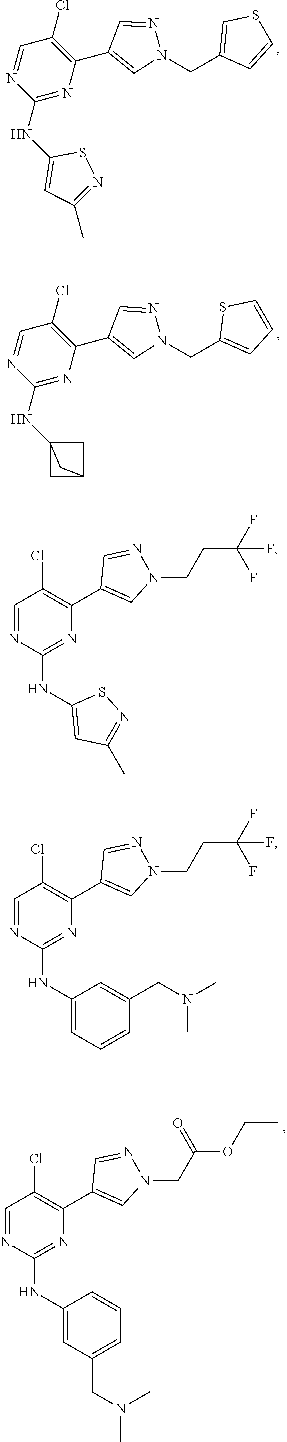 [SMILES: Cc1cc(Nc2ncc(Cl)c(-c3cnn(Cc4ccsc4)c3)n2)sn1 ; Clc1cnc(NC23CC(C2)C3)nc1-c1cnn(Cc2cccs2)c1 ; Cc1cc(Nc2ncc(Cl)c(-c3cnn(CCC(F)(F)F)c3)n2)sn1 ; CN(C)Cc1cccc(Nc2ncc(Cl)c(-c3cnn(CCC(F)(F)F)c3)n2)c1 ; CCOC(=O)Cn1cc(-c2nc(Nc3cccc(CN(C)C)c3)ncc2Cl)cn1] 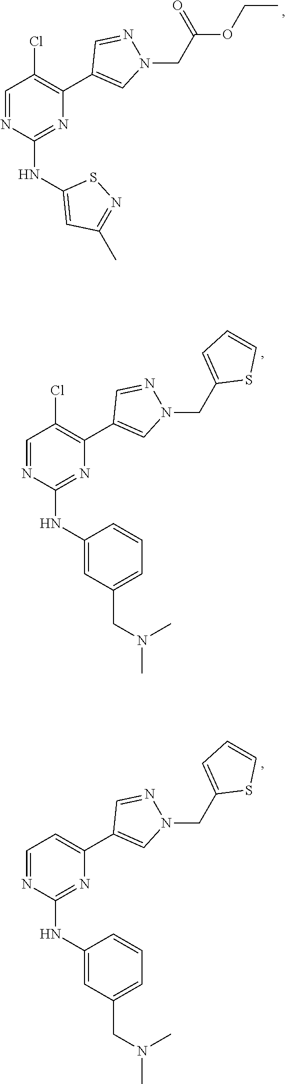 [SMILES: CCOC(=O)Cn1cc(-c2nc(Nc3cc(C)ns3)ncc2Cl)cn1 ; CN(C)Cc1cccc(Nc2ncc(Cl)c(-c3cnn(Cc4cccs4)c3)n2)c1 ; CN(C)Cc1cccc(Nc2nccc(-c3cnn(Cc4cccs4)c3)n2)c1] 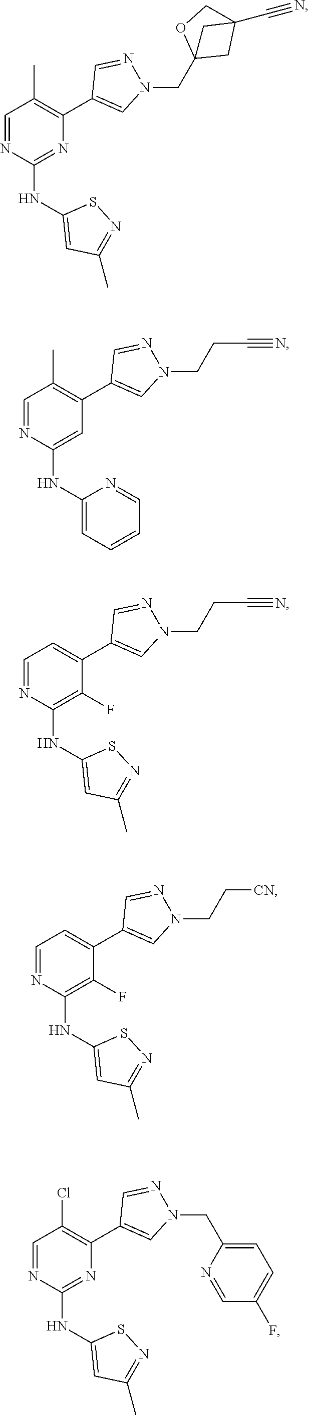 [SMILES: Cc1cc(Nc2ncc(C)c(-c3cnn(CC45CC(C#N)(CO4)C5)c3)n2)sn1 ; Cc1cnc(Nc2ccccn2)cc1-c1cnn(CCC#N)c1 ; Cc1cc(Nc2nccc(-c3cnn(CCC#N)c3)c2F)sn1 ; Cc1cc(Nc2nccc(-c3cnn(CCC#N)c3)c2F)sn1 ; Cc1cc(Nc2ncc(Cl)c(-c3cnn(Cc4ccc(F)cn4)c3)n2)sn1] 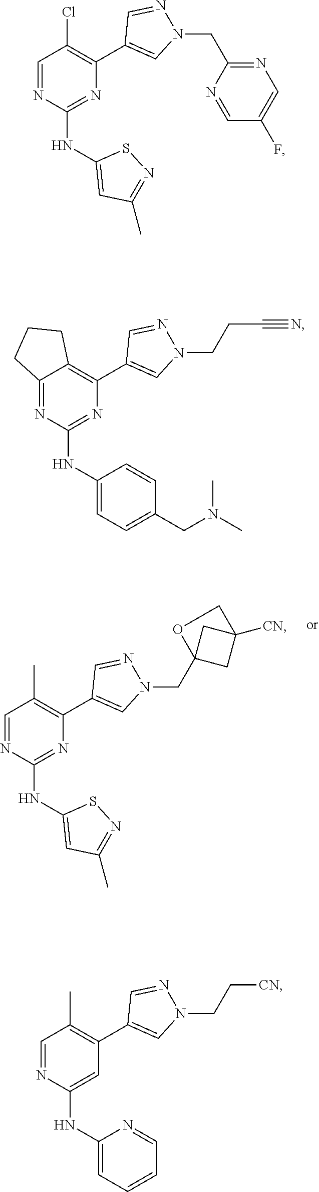 [SMILES: Cc1cc(Nc2ncc(Cl)c(-c3cnn(Cc4ncc(F)cn4)c3)n2)sn1 ; CN(C)Cc1ccc(Nc2nc3c(c(-c4cnn(CCC#N)c4)n2)CCC3)cc1 ; Cc1cc(Nc2ncc(C)c(-c3cnn(CC45CC(C#N)(CO4)C5)c3)n2)sn1 ; "or" ; Cc1cnc(Nc2ccccn2)cc1-c1cnn(CCC#N)c1]
or a pharmaceutically acceptable salt thereof.

17. A compound selected from 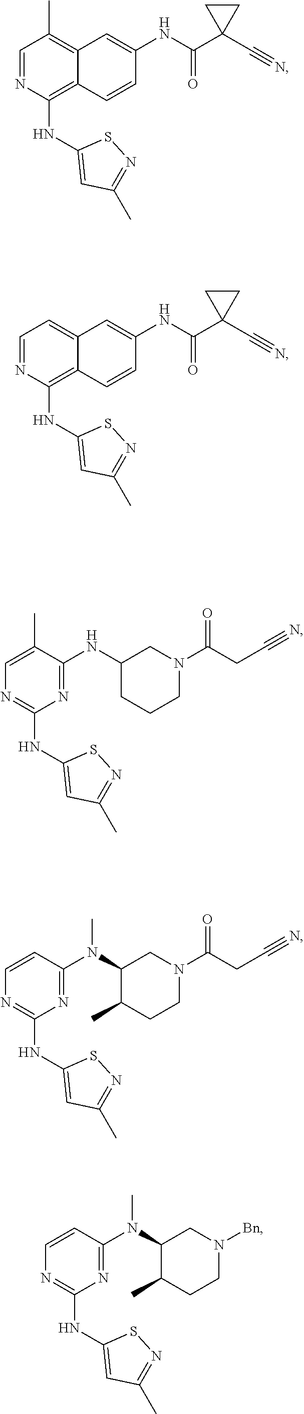 [SMILES: Cc1cc(Nc2ncc(C)c3cc(NC(=O)C4(C#N)CC4)ccc23)sn1 ; Cc1cc(Nc2nccc3cc(NC(=O)C4(C#N)CC4)ccc23)sn1 ; Cc1cc(Nc2ncc(C)c(NC3CCCN(C(=O)CC#N)C3)n2)sn1 ; Cc1cc(Nc2nccc(N(C)[C@H]3CN(C(=O)CC#N)CC[C@H]3C)n2)sn1 ; Cc1cc(Nc2nccc(N(C)[C@H]3CN(Cc4ccccc4)CC[C@H]3C)n2)sn1] 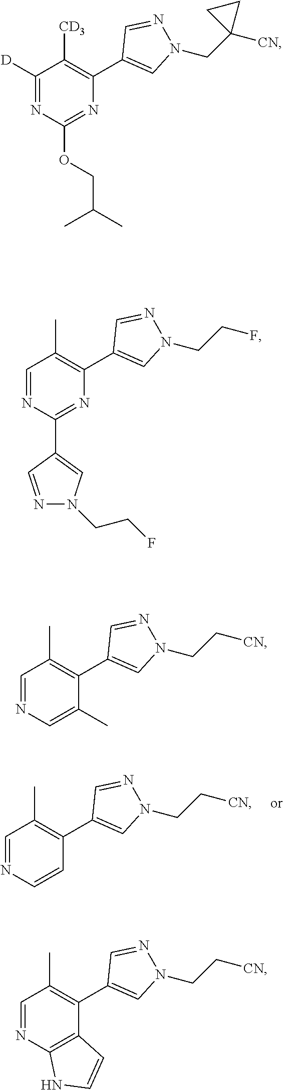 [SMILES: [2H]c1nc(OCC(C)C)nc(-c2cnn(CC3(C#N)CC3)c2)c1C([2H])([2H])[2H] ; Cc1cnc(-c2cnn(CCF)c2)nc1-c1cnn(CCF)c1 ; Cc1cncc(C)c1-c1cnn(CCC#N)c1 ; Cc1cnccc1-c1cnn(CCC#N)c1 ; Cc1cnc2[nH]ccc2c1-c1cnn(CCC#N)c1]
or a pharmaceutically acceptable salt thereof.

18. A compound of the formula: 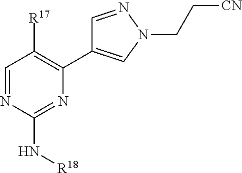
or a pharmaceutically acceptable salt thereof,
wherein
R¹⁷ is H, Cl, or CH₃; and
R¹⁸ is

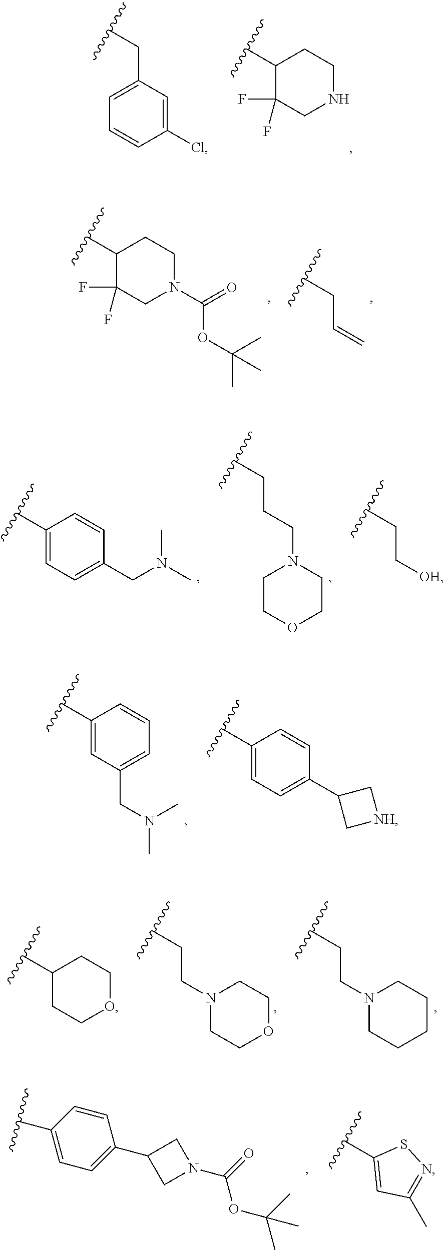

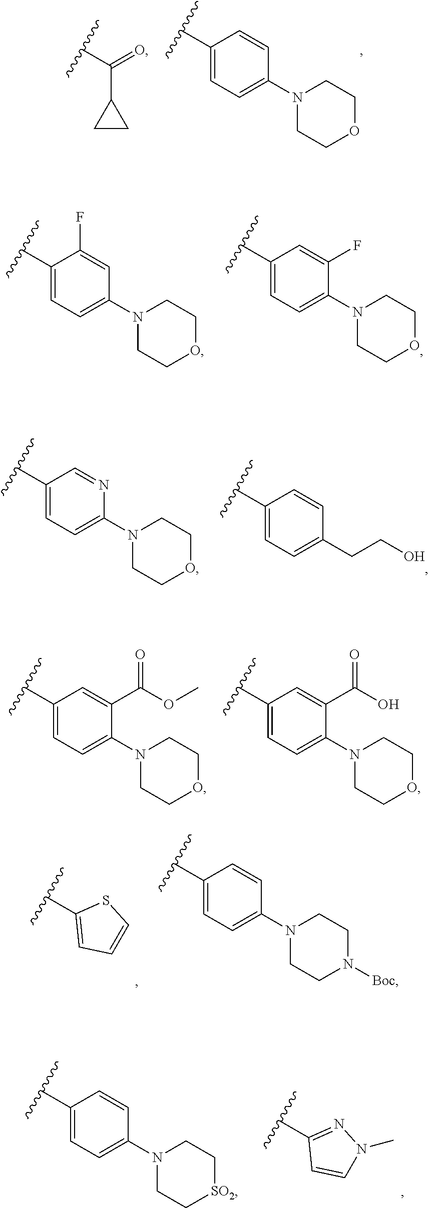

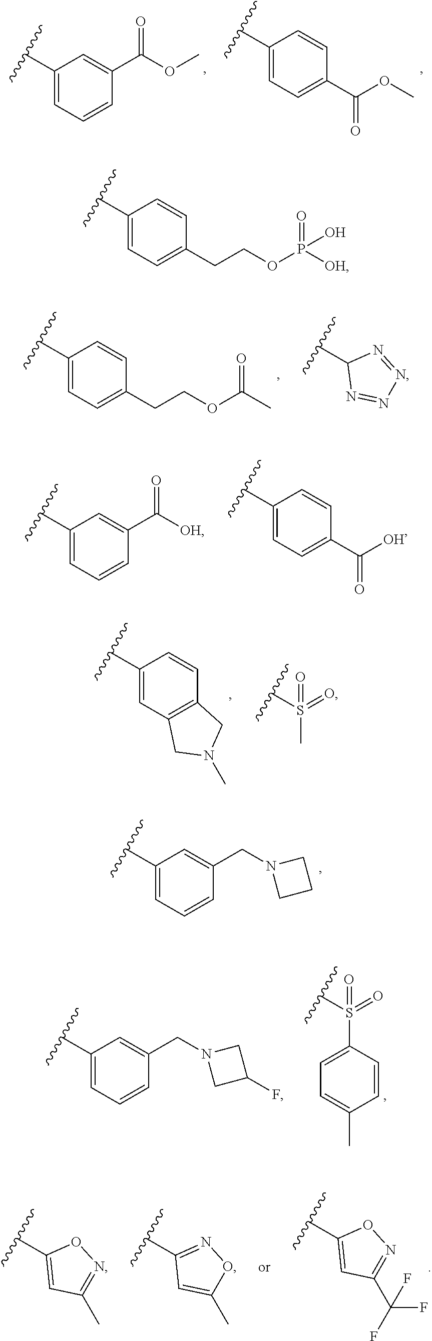

19. The compound of claim 18, wherein the compound is 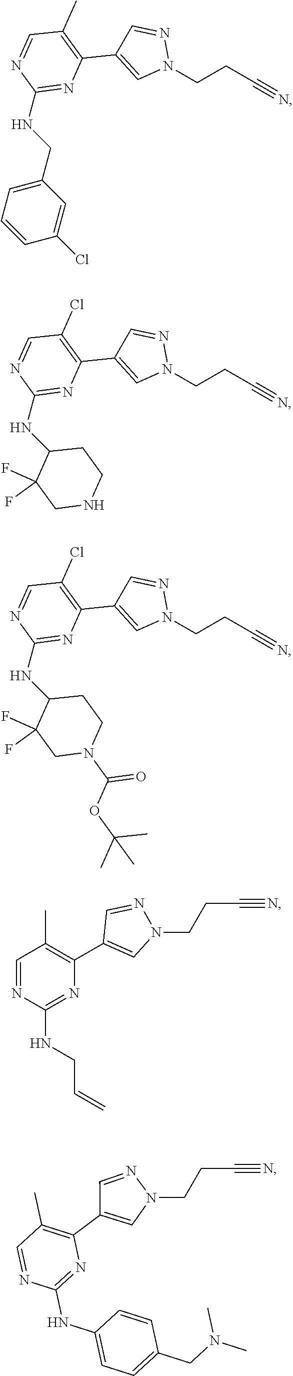 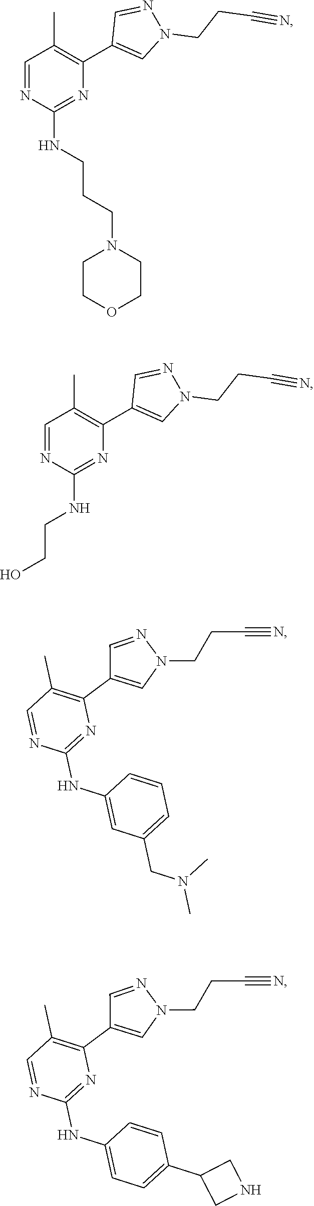 [SMILES: Cc1cnc(NCCCN2CCOCC2)nc1-c1cnn(CCC#N)c1 ; Cc1cnc(NCCO)nc1-c1cnn(CCC#N)c1 ; Cc1cnc(Nc2cccc(CN(C)C)c2)nc1-c1cnn(CCC#N)c1 ; Cc1cnc(Nc2ccc(C3CNC3)cc2)nc1-c1cnn(CCC#N)c1] 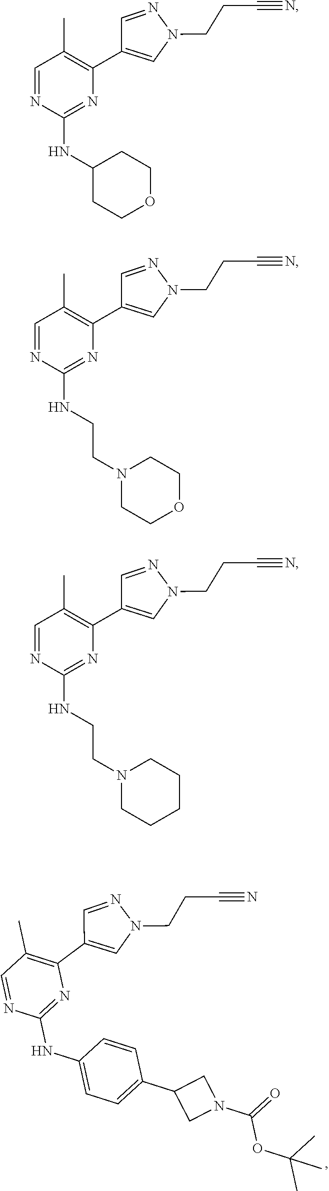 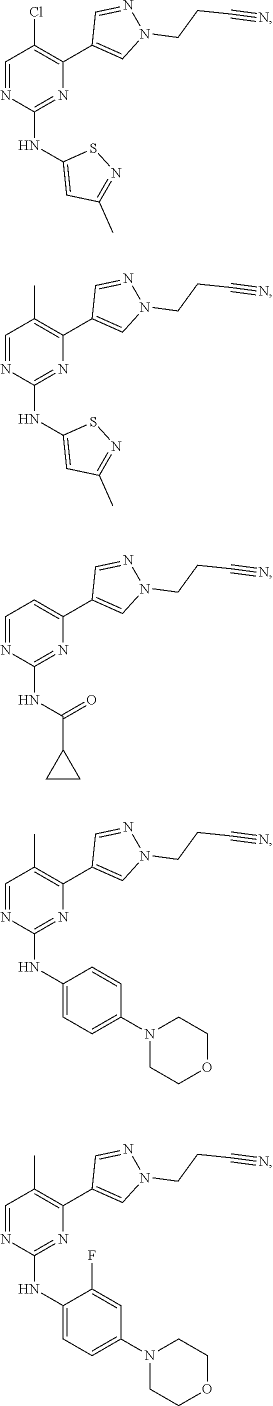 [SMILES: Cc1cc(Nc2ncc(Cl)c(-c3cnn(CCC#N)c3)n2)sn1 ; Cc1cc(Nc2ncc(C)c(-c3cnn(CCC#N)c3)n2)sn1 ; N#CCCn1cc(-c2ccnc(NC(=O)C3CC3)n2)cn1 ; Cc1cnc(Nc2ccc(N3CCOCC3)cc2)nc1-c1cnn(CCC#N)c1 ; Cc1cnc(Nc2ccc(N3CCOCC3)cc2F)nc1-c1cnn(CCC#N)c1] 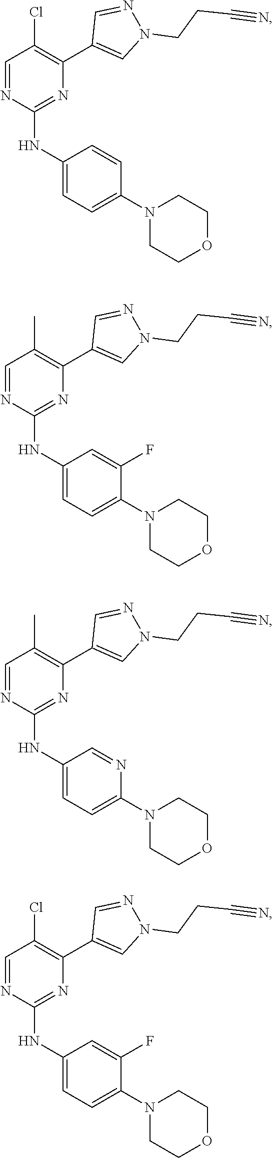 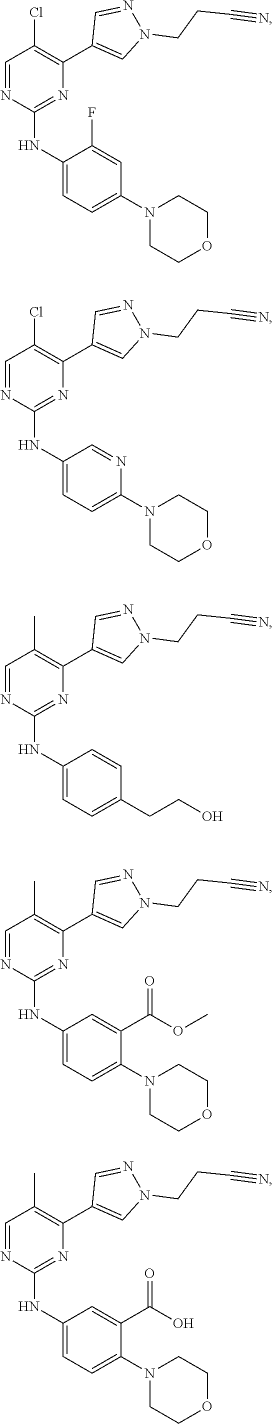 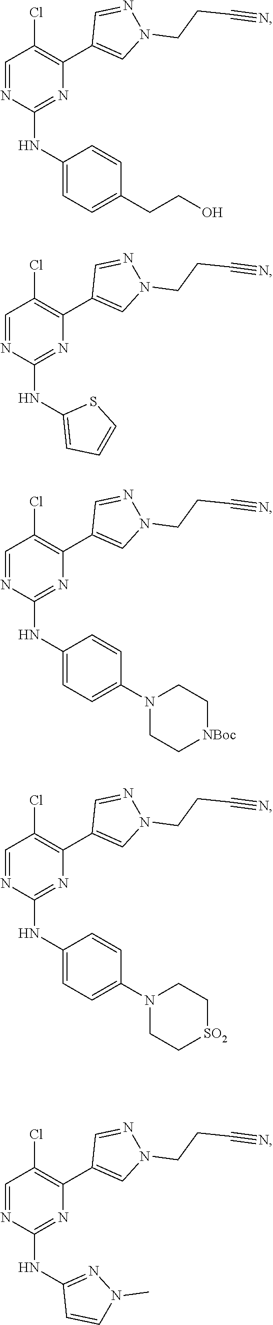 [SMILES: N#CCCn1cc(-c2nc(Nc3ccc(CCO)cc3)ncc2Cl)cn1 ; N#CCCn1cc(-c2nc(Nc3cccs3)ncc2Cl)cn1 ; CC(C)(C)OC(=O)N1CCN(c2ccc(Nc3ncc(Cl)c(-c4cnn(CCC#N)c4)n3)cc2)CC1 ; N#CCCn1cc(-c2nc(Nc3ccc(N4CCS(=O)(=O)CC4)cc3)ncc2Cl)cn1 ; Cn1ccc(Nc2ncc(Cl)c(-c3cnn(CCC#N)c3)n2)n1] 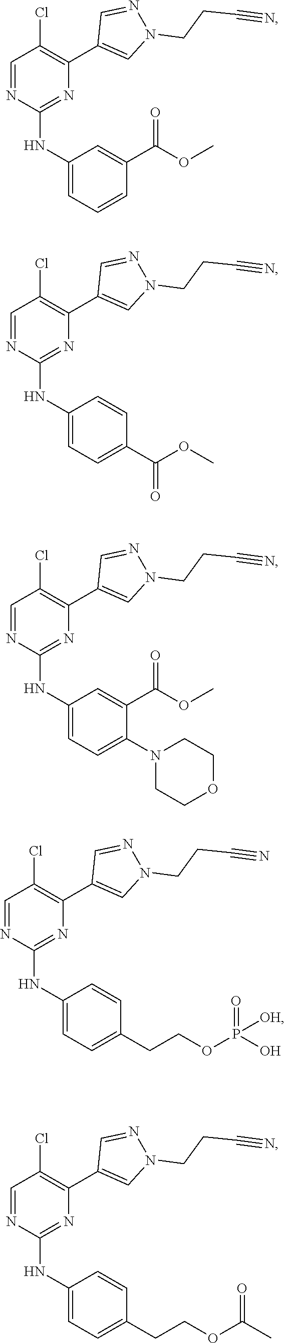 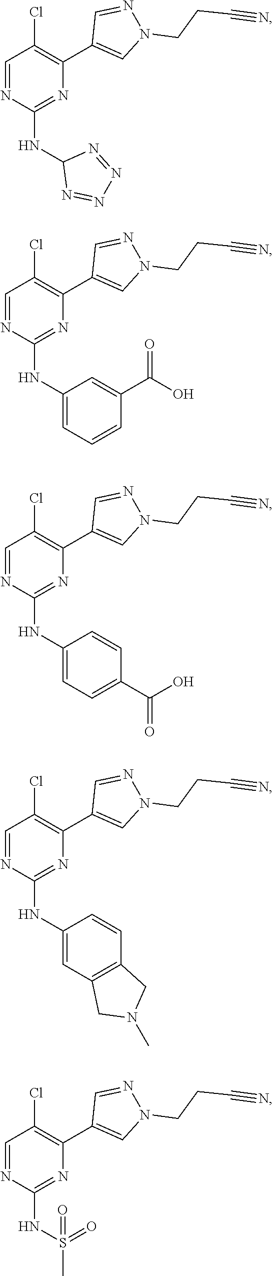 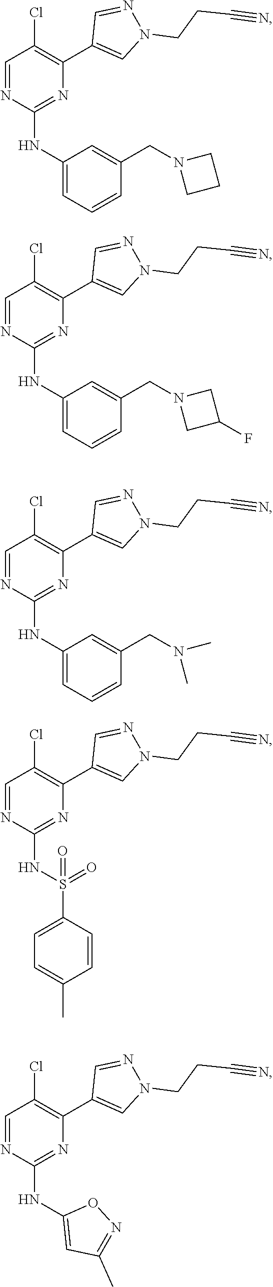 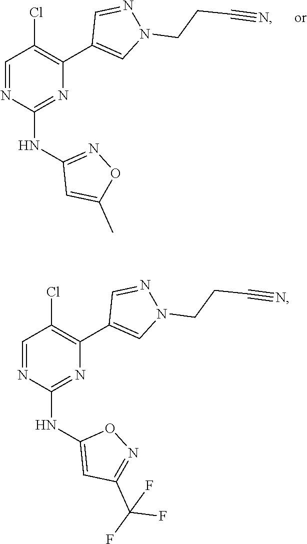 [SMILES: Cc1cc(Nc2ncc(Cl)c(-c3cnn(CCC#N)c3)n2)no1 ; N#CCCn1cc(-c2nc(Nc3cc(C(F)(F)F)no3)ncc2Cl)cn1]
or a pharmaceutically acceptable salt thereof.

20. A compound selected from 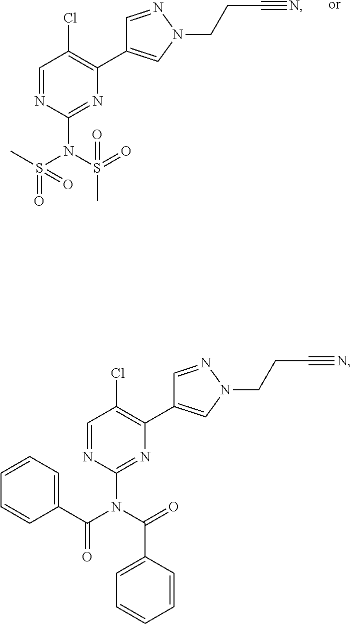 [SMILES: CS(=O)(=O)N(c1ncc(Cl)c(-c2cnn(CCC#N)c2)n1)S(C)(=O)=O ; "or" ; N#CCCn1cc(-c2nc(N(C(=O)c3ccccc3)C(=O)c3ccccc3)ncc2Cl)cn1]
or a pharmaceutically acceptable salt thereof.

21. A composition, comprising the compound of any one of claims 1-20.

22. The composition of claim 21, wherein the composition is a pharmaceutical composition, further including a pharmaceutically acceptable carrier.

23. A method of treating a disease in a subject in need thereof, comprising administering a therapeutically effective amount of the compound of one of claims 1-20, or the composition of one of claims 21-22, to the subject.

24. The method of claim 23, wherein the disease is associated with modulation of kinase activity.

25. The compound of one of claims 1-20, or the composition of one of claims 21-22, wherein the compound or composition is housed within a container.

26. A method, comprising administering the compound of one of claims 1-20, or the composition of one of claims 21-22, to a subject.

27. A method, comprising contacting a Janus kinase with the compound of one of claims 1-20, or the composition of one of claims 21-22, optionally wherein the Janus kinase is in a subject.
